SEQUENCE LISTING (14 sequences)

SEQ ID NO: 1 - DNA, 40 bp, Artificial Sequence
  DNA 3WJ
cgacaaaatgcaaaagcattacttcaaaagaagtttgtcg

SEQ ID NO: 2 - DNA, 10 bp, Artificial Sequence
  dsDNA
ccagtactgg

SEQ ID NO: 3 - DNA, 20 bp, Artificial Sequence
  Synthetic hairpin DNA
caaaatgcaaaagcattttg

SEQ ID NO: 4 - DNA, 38 bp, Artificial Sequence
  DNA 3WJ2
gggagacaaggaaaatccttcaatgaagtgggtcgaca

SEQ ID NO: 5 - DNA, 12 bp, Artificial Sequence
  Synthetic inhibitor strand
tccttgtctccc

SEQ ID NO: 6 - RNA, 38 nt, Artificial Sequence
  RNA 3WJ2
ggcacaaaugcaacacugcauuaccaugcgguugugcc

SEQ ID NO: 7 - RNA, 38 nt, Artificial Sequence
  mRNA model system
ggcacaaacgcaacacugcauuaccaugcgguugugcc

SEQ ID NO: 8 - DNA, 16 bp, Artificial Sequence
  Synthetic inhibitor 16
gtgttgcatttgtgcc

SEQ ID NO: 9 - DNA, 30 bp, Artificial Sequence
  Synthetic primer
gatctagaatcgattgagaggatttgaatg

SEQ ID NO: 10 - DNA, 29 bp, Artificial Sequence
  Synthetic primer
gagaattcccgcctgtggcaggccatagc

SEQ ID NO: 11 - DNA, 29 bp, Artificial Sequence
  Synthetic primer
gatctagagaacttgtggataaaatcacg

SEQ ID NO: 12 - DNA, 34 bp, Artificial Sequence
  Synthetic primer
gagaattcggatccttacgcttcaatggcagcac

SEQ ID NO: 13 - DNA, 39 bp, Artificial Sequence
  TNR DNA 3WJ
gcggagcagcccttgggcagcaccttggtgctgctccgc

SEQ ID NO: 14 - DNA, 10 bp, Artificial Sequence
  Synthetic inhibitor 10
gctgctccgc

(19) United States
(12) Patent Application Publication
(10) Pub. No.: US 2018/0265911 A1
(43) Pub. Date: Sep. 20, 2018

(54) TRIPTYCENE DERIVATIVES FOR NUCLEIC ACID JUNCTION STABILIZATION

(71) Applicant: THE TRUSTEES OF THE UNIVERSITY OF PENNSYLVANIA, Philadelphia, PA (US)

(72) Inventors: David M. CHENOWETH, Philadelphia, PA (US); Stephanie A. BARROS, Philadelphia, PA (US)

(21) Appl. No.: 15/762,362

(22) PCT Filed: Sep. 26, 2016
(86) PCT No.: PCT/US2016/053801

§ 371 Date: Mar. 22, 2018

Related U.S. Application Data
(60) Provisional application No. 62/232,324, filed on Sep. 24, 2015.

(51) Int. Cl. C12Q 1/68; G01N 33/50; C12Q 1/02; C07C 271/30

(52) U.S. Cl. CPC C12Q 1/68; G01N 33/5008; C07C 2603/90; C07C 271/30; C12Q 1/025

(57) ABSTRACT

The present invention is directed to compositions and methods using triptycene derivatives (TCDs) for three way junctions (TWJs).

### CROSS REFERENCE TO RELATED APPLICATIONS

The present application claims benefit of priority to US Provisional Patent Application No. 62/232,324, filed Sep. 24, 2015, which is incorporated by reference.

### BACKGROUND OF THE INVENTION

Despite significant advances in modern medicine and drug discovery, there are still tremendous unmet medical needs for combating human diseases.

Nucleic acid junctions are ubiquitous in biological systems. Small molecule control of these structures would allow for regulation of a myriad of nucleic acid dependent biological processes. In addition, these probes will provide fundamental insight into biological systems. Small molecule nucleic acid junction binders could open new avenues for the discovery and development of therapeutics to address unmet medical needs.

However, targeting DNA and RNA structure for therapeutic use has been hampered by an incomplete understanding of the requirements needed for small molecule recognition in a structure and sequence specific manner. To date, the most popular strategies for targeting nucleic acid structures over the last few decades are to make use of known binding modes, utilizing fragments of known intercalating and groove binding ligands or structural modification of natural products such as the aminoglycoside antibiotics. Despite this progress, there are few classes of molecules with the ability to target DNA and RNA motifs in a structure and sequence specific manner.

Significant unmet medical needs necessitate the development of new strategies for combating infectious diseases and neurodegenerative diseases. Alternative strategies to combat life-threatening illness from infectious agents such as drug resistant bacteria and viruses are desperately needed as resistance builds and our current therapies begin to fail. Neurodegenerative disease will become a significant burden with our aging population. Small molecule nucleic acid regulation could have a significant impact in both of these diverse areas and provide new tools for elucidating the fundamental biology. Viral, bacterial, and parasitic diseases contribute to global health and economic problems of significant magnitude.

Nucleic acid and Nucleic acid-protein interactions represent examples of non-traditional drug targets with significant potential in nearly all areas of human medicine. This is primarily due to the central role of nucleic acids in a diverse array of biological processes. For example, RNA is involved in a multitude of vital biological processes ranging from information transfer (mRNA) and gene regulation (siRNA's and microRNA's) to catalysis (ribozymes and riboswitches). The siRNA pathway and the diverse world of non-coding RNA's have regulatory functions ranging from cellular differentiation and chromosomal organization to the regulation of gene expression. The ability to target RNA-dependent processes in bacterial and viral pathogens in addition to pathogenic RNAs implicated in neurological diseases and cancer represent important challenges. Currently, we lack the ability to design molecules to target important DNA and RNA structural motifs beyond B-form DNA with high affinity and specificity. A clear set of rules and guidelines for targeting specific, non-B-form DNA, nucleic acid structural motifs is non-existent and the ability to design small molecules from secondary structure prediction is in its infancy. Given the vital role of nucleic acids in biological processes, a guide to targeting specific structural elements could have far reaching impacts in areas ranging from fundamental biology to new strategies for combating human disease.

Nucleic acid junctions are ubiquitous structural elements present in prokaryotes and eukaryotes. Three-way junctions (3WJ or 3HSm junctions) are formed at the interface of three double helical nucleic acids, forming a Y-shape junction, with a hydrophobic cavity in the center. DNA 3WJs are present in important biological processes, including replication and recombination. They are also present in trinucleotide repeat expansions associated with neurodegenerative diseases and occur in viral genomes. RNA 3WJs are found in a number of important RNA targets including the IRES of the hepatitis C virus (HVC), the hammered ribozyme and in bacterial temperature sensors such as the mRNA of sigma32 (σ³²) in E. Coli. Advances towards targeting nucleic acids in a structure-specific manner remain a challenge. The ability to selectively target these junctions would allow for the precise control of cellular processes at the nucleic acid level.

The development of nucleic acid binding small molecules is a major challenge with great potential. To date, there are just a handful of commonly recognized nucleic acid binding modes: minor groove binding, major groove binding, intercalation, and phosphate backbone recognition in addition to several hybrid binding manifolds that rely on simultaneous intercalation and groove binding. The threading intercalators are a prime example along with several natural products that utilize multiple simultaneous binding events to increase their overall affinity and sometimes specificity. The Py-Im polyamides are perhaps one of the most successful platforms for nucleic acid recognition although they are primarily limited to targeting dsDNA structure and have not been shown to be effective at targeting RNA structure. Polyamides are selective for dsDNA over dsRNA, primarily due to an absence of a deep narrow minor groove in RNA structure. Selective and sequence specific targeting of unique DNA structures beyond the double helix has not been demonstrated. Utilizing fragment-based approaches to target RNA structure is an area beginning to show promise. These approaches often build upon common nucleic acid binding scaffolds such as the aminoglycosides, Hoechst, polyamides, known intercalators, or polyamides in addition to polyvalent peptide, peptoid and polymeric scaffolds often bearing multiple cationic functional groups. Minor groove binding ligands have also been reported to bind nucleic acid junctions; however, there is a lack of specificity over binding double helical nucleic acid structures. Intercalators have been broadly utilized to target both DNA and RNA structure however this approach also suffers from a lack of specificity.

There is a minimal amount of precedent for targeting nucleic acid three-way junctions; however, to date all prior studies utilize molecular scaffolds that are already known to bind non-junction nucleic acid structures leading to little selectivity for their intended target. Nucleic acid junctions have been targeted with poly-intercalators with the major limitation again being non-specific intercalation events leading to promiscuity. Cationic peptides have been extensively utilized to target nucleic acid structure and a particularly interesting example by Segall demonstrated the use in modulating holiday junctions (four-way junctions) for the inhibition of junction processing enzymes. It should be noted that the approach of Segall is fundamentally different from the approach proposed in this invention. In addition, the holiday junctions previously targeted by Segall unique structures in their own right are distinct from the three-way junctions targeted in the present invention. Hannon, Coll, and co-workers have demonstrated that metal helicates bind to many nucleic acid structures including quadruplexes and other helical motifs. Different from the metal helicates in the literature, the triptycenes are a novel class of small molecule for targeting nucleic acid junctions.

### BRIEF SUMMARY OF THE INVENTION

In one aspect, the present disclosure provides a method of screening for triptycene derivative (TCD) compounds that stabilize a target nucleic acid three way junction (TWJ) structures comprising:
a) providing an array comprising a solid support comprising a plurality of assay locations each comprising a covalently attached different TCD;
b) contacting said array with a target TWJ comprising:
   i) a nucleic acid substrate with an attached fluorophore donor and an attached fluorophore acceptor; and
   ii) a nucleic acid inhibitor that hybridizes to said substrate to form an inhibitor complex such that said donor and acceptor are separated and FRET does not occur, wherein said contacting is done under conditions wherein one of said TCDs binds to said TWJ such that said inhibitor is released and that FRET occurs; and
c) determining the binding of said TCD to said TWJ by detecting the presence or absence of FRET.

In one aspect, the present disclosure provides a method of screening for triptycene derivative (TCD) compounds that stabilize a target nucleic acid three way junction (TWJ) structures comprising:
a) providing a target nucleic acid substrate with an attached fluorophore donor and an attached fluorophore acceptor, said substrate forming a TWJ such that said donor and acceptor undergo fluorescence resonance energy transfer (FRET);
b) contacting said substrate with a nucleic acid inhibitor that hybridizes to said substrate to form an inhibitor complex such that said donor and acceptor are separated and FRET does not occur;
c) contacting said inhibited complex with a triptycene derivative (TCD) under conditions wherein said inhibitor is released and said TCD binds to said TWJ such that FRET reoccurs; and
d) determining the binding of said TCD to said TWJ by detecting the presence or absence of FRET.

In one aspect, the present disclosure provides a method of screening for triptycene derivative (TCD) compounds that stabilize nucleic acid three way junction (TWJ) structures comprising:
a) providing a nucleic acid substrate with an attached first fluorophore that forms a TWJ;
b) contacting said substrate with a nucleic acid inhibitor that hybridizes to said substrate to prevent the formation of the TWJ to form an inhibited complex, wherein said inhibitor comprises an attached second fluorophore, wherein said first and second fluorophore will undergo FRET when said inhibitor complex is formed;
c) contacting said inhibited complex with a TCD under conditions wherein said inhibitor is released, and said TCD binds to said TWJ such that FRET does not occur; and
d) determining the binding of said TCD to said TWJ by detecting the presence or absence of FRET.

In one embodiment of the methods provided herein, the nucleic acid substrate is contacted with a plurality of TCDs.

In one embodiment of the methods provided herein, the TCDs have the structure:

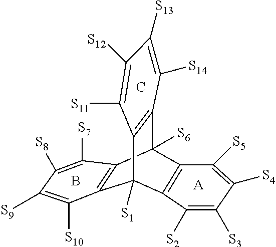

wherein each of S1 to S14 is independently and optionally selected from the group consisting of a hydrogen, substituted or unsubstituted alkyl, substituted or unsubstituted heteroalkyl, substituted or unsubstituted aryl, substituted or unsubstituted heteroaryl, halogen, CN, CF3, acyl, an amino acid analog, a peptide (including peptide analogs), a nucleotide (including nucleotide analogs) and an oligonucleotide (including oligonucleotide analogs), and wherein at least one S group is a non-hydrogen group.

In one embodiment of the methods provided herein, at least one of the S groups is an amino acid.

In one embodiment of the methods provided herein, at least one of the S groups is an amino acid analog.

In one embodiment of the methods provided herein, at least one of the S groups is a peptide.

In one embodiment of the methods provided herein, at least one of the S groups is a peptide analog.

In one embodiment of the methods provided herein, at least one of the S groups is a nucleotide.

In one embodiment of the methods provided herein, at least one of the S groups is a nucleotide analog.

In one embodiment of the methods provided herein, at least one of the S groups is an oligonucleotide.

In one embodiment of the methods provided herein, at least one of the S groups is an oligonucleotide analog.

In one embodiment of the methods provided herein, the TCD is covalently attached to a solid support.

In one embodiment of the methods provided herein, a plurality of different TCDs are attached at different sites to said solid support in an array pattern.

In one aspect, the present disclosure provides a method of screening for a cytotoxic TCD comprising contacting said TCD with a cell and determining the viability of said cell.

In one embodiment of the methods provided herein, the cell is a mammalian cell.

In one embodiment of the methods provided herein, the TCD is contacted with a healthy cell or a cancerous cell.

In one embodiment of the methods provided herein, the cell is a bacterial cell.

In one aspect, the present disclosure provides a method of screening for a TCD that inhibits viral replication comprising contacting a cell hosting a virus and determining the viability of the virus.

In one aspect, the present disclosure provides a composition comprising a solid support comprising an array of different TCDs.

In one embodiment of the composition provided herein, each TCD has the structure:

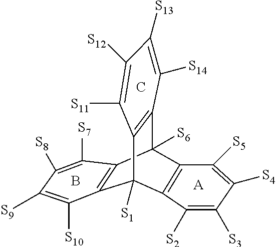

wherein each of S1 to S14 is independently and optionally selected from the group consisting of a hydrogen, substituted or unsubstituted alkyl, substituted or unsubstituted heteroalkyl, substituted or unsubstituted aryl, substituted or unsubstituted heteroaryl, halogen, CN, CF3, acyl, an amino acid analog, a peptide (including peptide analogs), a nucleotide (including nucleotide analogs) and an oligonucleotide (including oligonucleotide analogs), and wherein at least one S group is used to covalently attached said TCD to said array.

In one embodiment of the composition provided herein, at least one of the S groups is attached via an amido group.

In one embodiment of the composition provided herein, at least one of the S groups is an amino acid.

In one embodiment of the composition provided herein, at least one of the S groups is an amino acid analog.

In one embodiment of the composition provided herein, at least one of the S groups is a peptide.

In one embodiment of the composition provided herein, at least one of the S groups is a peptide analog.

In one embodiment of the composition provided herein, at least one of the S groups is a nucleotide.

In one embodiment of the composition provided herein, at least one of the S groups is a nucleotide analog.

In one embodiment of the composition provided herein, at least one of the S groups is an oligonucleotide.

In one embodiment of the composition provided herein, at least one of the S groups is an oligonucleotide analog.

### BRIEF DESCRIPTION OF THE DRAWINGS

While the invention will be described in conjunction with the following figures in order to explain certain principles of the invention and their practical applications. It will be understood that the present description is not intended to limit the invention(s) to those Figures or examplary embodiments. On the contrary, the invention(s) is/are intended to cover not only the exemplary embodiments as represented by the figures, but also various alternatives, modifications, equivalents and other embodiments, which are included within the spirit and scope of the invention as defined by the appended claims.

FIGS. 1A, 1B and 1C show the structure of non-substituted triptycene (TC) in FIG. 1A. FIG. 1B shows the possible locations for substituent groups, as defined herein. R1 groups are referred to herein as ring substituents and R2 are referred to herein as bridgehead substituents. FIG. 1C is labeled by substituent positions (S1 to S14) for unique identification and discussion herein.

FIG. 2: Examples of important nucleic acid three-way junctions.

FIG. 3: Triptycene-based scaffold developed in the Chenoweth laboratory for nucleic acid junction targeting. (Top Left) Model of triptycene/DNA-3WJ complex based on a crystal structure of trimeric Cre recombinase bound to a three-way Lox DNA junction (PDB ID: 1F44). (Bottom Left) Structure of triptycenes 1-3 from our initial studies. (Top Right) Concept graphic showing our fluorescence allosteric inhibitor displacement assay developed in our initial studies. (Bottom Right) Inhibitor displacement curves for triptycenes 1-3 using the fluorescence assay described above.

FIG. 4: Dynamic slipped DNA junctions formed by (CAG)⋅(CTG) repeats. It should be noted that a junction may not pre-exist prior to interaction with a triptycene or its derivative. The triptycene can induce formation of a junction that was not present prior to interaction with a triptycene or other small molecule.

FIG. 5: Route used to synthesize compound 1 for preliminary studies.

FIG. 6: Model of triptycene located at a central binding pocket of a model RNA three-way junction. Functionalization at positions 2, 10, and 11 should enhance binding and facilitate substituent interactions with base-pair edges. It should be noted that a junction may be formed from more than one strand.

FIG. 7: One library of natural and unnatural amino acid side chains.

FIG. 8: Synthesis of 2,10,11-substitution pattern on triptycene core.

FIG. 9: Triptycene building blocks and immobilization strategy. Note that in this embodiment, attachment is done using a bridgehead (R2) substituent to attach to a solid support, although R1 groups can be used as well.

FIG. 10: Junction recognition at putative binding site.

FIG. 11: General scheme for targeting sigma 32 in E. coli.

FIG. 12: Targeting sigma32 in E. coli. (Left) General scheme for targeting sigma 32 in E. coli. (Right) Transcription of sigma 32 mRNA. (Right) Schematic for sigma32/emGFP reporter plasmid. (Middle) Transcription of sigma 32 mRNA performed in our laboratory and reporter assay preliminary result.

FIG. 13: Covalent and non-covalent pull-down probes for targeting sigma 32 mRNA in E. coli.

FIGS. 14A and 14B depict two different assay formats for use herein. FIG. 14A shows the use of the target substrate 100 with a covalently attached Förster resonance energy transfer (FRET) donor and acceptor (105 and 106). As will be appreciated by those in the art, the position of the donor and acceptor can vary, e.g. the donor can be on the 3′ end or the 5′ end of the target nucleic acid substrate with the acceptor on the other, or vice versa. In this embodiment, the donor and acceptor undergo FRET and thus are considered “quenched”, referred to in FIG. 14 as “off”. A nucleic acid inhibitor (of varying lengths, as described below) is used that is long enough to thermodynamically favor the formation of the inhibitor complex (130) over the substrate TWJ under the conditions of the assay, in the absence of a TCD. In the inhibited complex, the donor and acceptor are now spatially separated, such that no significant FRET occurs, thus turning the complex “on”. A TCD (120) is added, such that now the stabilization of the TWJ favors the reformation of the TWJ substrate, thus turning the substrate back “on”. Thus by determining the presence or absence of FRET, the binding of the TCD to the TWJ can be measured. FIG. 14B is similar, except that one label of the FRET pair is on the inhibitor, not on the substrate, such that this system goes from unquenched with no inhibitor (“on”) to quenched with inhibitor (“off”) to unquenched with TWJ (“on”) again.

FIG. 15 depict two different possible attachment sites for attachment to a solid support as discussed herein. FIG. 15 (left) shows attachment through a bridgehead position, and FIG. 15 (right) shows attachment through the S10 position, although as will be appreciated by those in the art, any of positions S2 to S5, S7 to S10 and S11 to S14 can be used for non-bridgehead attachment.

FIG. 16 shows one type of screening assay of the present invention, where a library of TCDs are tested against a single TWJ. An array of different TCDs are made using the techniques outlined herein and attached to a solid support (or they can be synthesized on the support, as discussed herein). The TWJ is added in complex with the inhibitor, in either the “on” or “off” FRET status as discussed in FIG. 14, to render a difference in FRET status upon binding of the TCD to the substrate.

FIG. 17 shows one type of screening assay of the invention, where a single TCD is tested against a library of different TWJs to elucidate potential sequence specificity.

FIG. 18 shows a general schematic of a matrix type of screening assay of the present invention (library of TCDs and library of TWJs). An array of different TCDs is made using the techniques outlined herein, and attached to a solid support (or they can be synthesized on the support, as discussed herein). Then a library of different TWJs, all different in sequence, with sequence specific inhibitors are added to the solid support for a time period sufficient to allow the TCDs to bind to the TWJs, and the unbound TWJs are washed away. The solid support is then “read” to detect a change in fluorescence due to either the presence or absence of FRET (depending on the format as shown in FIG. 14), and any TWJs that are bound can then be released and detected (for example they can be sequenced or detected using sequence specific hybridization.

FIG. 19 shows the temperature-dependant circular dichroism CD of model system RNA in the absence (a) and presence of Trip 1 (b) or Trip 2 (c) as described in Example 2. Note that the designation of “Trip” with the same designation number may be different in different examples.

FIG. 20 shows modulation of σ³² mRNA (−19 to +229) by triptycene derivatives and targeting σ³² in E. coli in Example 2. a) UV thermal melting plots in the absence and presence of Trip 1 and Trip 2. b) Targeting rpoH using a σ³²-GFP fusion protein. c) Relative fluorescence intensities of the GFP control and the σ³²-GFP fusion protein at 30° C. and 42° C. in the presence and absence of Trip 1 and Trip 2.

FIG. 21 shows the temperature-dependant circular dichroism CD of rpoH RNA (−19 to +229) in the absence (a) and presence of Trip 1 (b) or Trip 2 (c) according to Example 2. Note that the designation of “Trip” with the same designation number may be different in different examples.

FIG. 22 shows a) The heat shock response in E. coli and a strategy for small molecule modulation at the mRNA level of Example 2. b) The overall secondary structure of the 5′-end of the σ³² mRNA regulatory element. Important regions are shown, with the boxed area corresponding to the AUG start codon of Example 2.

FIG. 23 shows stabilization of a model-system RNA by triptycene derivatives 1 and 2 in Example 2. a) Structures of the triptycene derivatives Trip 1 and Trip 2. b) The RNA oligonucleotide used as a model system, corresponding to a minimal sequence for junction formation. c) UV thermal melting plots in the presence and absence of the triptycenes. d) Schematic representation of the fluorescence quenching experiment. e) Titration of inhibitor 16 (I16) results in an increase in fluorescence. f) Titration of Trip 1 or Trip 2 to the RNA*-I16 complex results in a decrease in fluorescence. The apparent K_{d} values of Trip 1 and Trip 2 were determined to be 2.5 mm and 1.5 mm, respectively.

FIG. 24: Triptycene scaffold for nucleic acid three-way junction targeting as outlined in Example 1. (a) Relative size comparison of triptycene bound to the putative DNA 3WJ binding pocket. Model complex based on a crystal structure of trimeric Cre recombinase bound to a three-way Lox DNA junction (PDB ID: 1F44) (b) Structure of triptycene derivatives (Trip 1-3) utilized for targeting nucleic acid junctions. Note that the designation of “Trip” with the same designation number may be different in different examples.

FIG. 25: Thermal stabilization data from Example 1. (a) Representative fraction folded plot derived from UV thermal melting experiments showing significant stabilization in the presence of triptycene 1. Plot was derived using standard fitting procedures. (b) Table of UV thermal stabilization data comparing dsDNA and DNA 3WJ. Compound 4 was used to demonstrate the unique ability of triptycene to act as a structure specific core. Hairpin DNA showed no significant stabilization in the presence of ligands. (c) Circular dichroism spectra of DNA 3WJ in the absence (left) and presence (right) of triptycene 1. (Sequences of oligonucleotides used in these studies are as follows: DNA 3WJ: 5′-CGA CAA AAT GCA AAA GCA TTA CTT CAA AAG AAG TTT GTC G-3′, dsDNA: 5′-CCAGTACTGG-3′, Hairpin DNA: 5′-CAA AAT GCA AAA GCA TTT TG-3′.) Note that the designation of “Trip” with the same designation number may be different in different examples.

FIG. 26: Fluorescence quench assay, thermal stability data, and gel shift data for Example 1. (a) Schematic of the fluorescence quench based competitive oligonucleotide inhibition assay. (b) Opening the three-way junction (3WJ2) with a complementary oligonucleotide (I12). Fluorescence increases with an increase in concentration of I12. (c) Closing of the three-way junction (3WJ2) and displacement of competitive inhibitor I12 with triptycenes 1, 2, and 3. Fluorescence decreases with an increase in concentration of triptycene. The observed Kd's for Trip 1, 2, and 3 were determined to be 0.221 μM, 0.396 μM, and 5.499 μM, respectively. (d) Fraction folded plot derived from UV thermal melting experiments showing significant stabilization in the presence of triptycene 1. (e) Gel shift data showing opening of the three-way junction DNA using a complementary oligonucleotide analogous to the fluorescence quench in 3b. (f) Displacement of the complementary oligonucleotide and reformation of the three-way junction upon titration of triptycene 1. (Sequences of oligonucleotides used in these studies are as follows: DNA 3WJ2: 5′-GGG AGA CAA GGA AAA TCC TTC AAT GAA GTG GGT CGA CA-3′, Inhibitor strand I12: 5′-TCC TTG TCT CCC-3′, doubled labeled oligo sequence matches that of 3WJ2). Note that the designation of “Trip” with the same designation number may be different in different examples.

FIG. 27: UV absorption spectra for Example 1. (a) UV absorption for triptycene 1. (b) UV absorption for triptycene 2. (b) UV absorption for triptycene 3. (c) UV absorption for compound 4. Note that the designation of “Trip” with the same designation number may be different in different examples.

FIG. 28: depicts a synthetic scheme of the invention.

FIG. 29 shows according to Example 1: (a) Minimum free energy structure of DNA 3WJ designed using NUPACK. (b) Predicted melting curve generated by NUPACK.

FIG. 30: Example 2 UV thermal stabilization data for 1 in 10 mM CacoK, pH 7.2. (a) Normalized plot from UV thermal melting experiment with DNA 3WJ at 1 μM in the absence (black) and presence of 1 (red). (b) Fraction folded plot for DNA 3WJ at 1 μM in the absence (black) and presence of 1 (red). (c) Normalized plot from UV thermal melting experiment with dsDNA at 2 μM. (d) Fraction folded plot for dsDNA in the absence (black) and presence (red).

FIG. 31 Example 1 UV thermal stabilization data for 1 at different concentrations in 10 mM CacoK, pH 7.2. (a) Normalized plot from UV thermal melting experiment with DNA 3WJ at 1 μM in the absence (black) and presence of 1 at 1, 2, and 4 equivalents (blue, green red, respectively). A double inflection is observed with 1 equivalent of 1, indicating that the compound is not completely bound. (b) Fraction folded plot for DNA 3WJ at 1 μM in the absence (black) and presence of 1 at 2 and 4 equivalents (green and red, respectively).

FIG. 32 shows Example 1 UV thermal stabilization data for triptycene 2. (a) Normalized plot from UV thermal melting experiment with DNA 3WJ at 1 μM in the absence (black) and presence of 2 (red). (b) Fraction folded plot for DNA 3WJ and 2. (c) Normalized plot from UV thermal melting with dsDNA at 2 μM in the absence (black) and presence of 2 (red). (d) Fraction folded plot for dsDNA and 2.

FIG. 33 depicts Example 1 UV thermal stabilization data for triptycene 3. (a) Normalized plot from UV thermal melting experiment with DNA 3WJ at 1 μM in the absence (black) and presence of 3 (red). (b) Fraction folded plot for DNA 3WJ and 3. (c) Normalized plot from UV thermal melting with dsDNA at 2 μM in the absence (black) and presence of 3 (red). (d) Fraction folded plot for dsDNA and 3.

FIG. 34 depicts Example 1 UV thermal stabilization data for control 4. (a) Normalized plot from UV thermal melting experiment with DNA 3WJ at 1 μM in the absence (black) and presence of 4 (red). (b) Fraction folded plot for DNA 3WJ and 4. (c) Normalized plot from UV thermal melting with dsDNA at 2 μM in the absence (black) and presence of 4 (red). (d) Fraction folded plot for dsDNA and 4.

FIG. 35 depicts Example 1 UV thermal stabilization data for triptycenes 1, 2, and 3 with a DNA hairpin. (a) Normalized plot from UV thermal melting experiment with DNA hairpin at 1 μM in the absence (black) and presence of 1 (red). (b) Fraction folded plot for DNA hairpin and 1. (c) Normalized plot from UV thermal melting with DNA hairpin at 1 μM in the absence (black) and presence of 2 (red). (d) Fraction folded plot for DNA hairpin and 2. (a) Normalized plot from UV thermal melting experiment with DNA hairpin at 1 μM in the absence (black) and presence of 3 (red). (b) Fraction folded plot for DNA hairpin and 3.

FIG. 36 depicts Example 1 Temperature-dependant CD spectra. CD spectra of 20 μM DNA 3WJ in 10 mM CacoK, pH 7.2 at different temperatures of DNA 3WJ in the absence (a) and presence (b) of triptycene 1.

FIG. 37 depicts Example 1 CD thermal experiment. (a) CD spectra of 50 μM DNA 3WJ in 10 mM CacoK, pH 7.2 in the absence (black) and presence of 1 (red). (b) Molar ellipticity at 255 nm as a function of temperature. Thermal analysis was run with 50 μM DNA 3WJ with (red) and without (black) 1.

FIG. 38 depicts Example 1 CD spectra of 20 μM DNA 3WJ2 in 10 mM CacoK, pH 7.2 at 4° C.

FIG. 39 depicts Example 1 UV thermal stabilization data for triptycene 1. (a) Normalized plot from UV thermal melting experiment with DNA 3WJ2 at 1 μM in the absence (black) and presence of 1 (red). (b) Fraction folded plot for DNA 3WJ2 and 1.

FIG. 40 depicts Example 1 UV thermal stabilization data for triptycenes 1, 2, and 3. (a) Normalized plot from UV thermal melting experiment with RNA 3WJ2 at 1 μM in the absence of ligand (black) and in the presence of 1 (blue), 2 (green), and 3 (red). (b) Fraction folded plot for RNA 3WJ2 at 1 μM in the absence of ligand (black) and in the presence of 1 (blue), 2 (green), and 3 (red). RNA sequence: 5′-GGCACAAAUGCAACACUGCAUUACCAUGCGGUUGUGCC-3′.

FIG. 41 depicts Example 1 Cell uptake studies using MALDI-MS. Cells were incubated with triptycenes for 2 hours, pelleted, washed with buffer three times to remove extracellular compound, and then lysed. Column 1: Spectra for Trip 1. (a) Spectrum of Trip 1 from wash 1. A m/z=768.203 was observed for the triptycene. (b) Spectrum of Trip 1 from wash 2. The mass of Trip 1 is still present at a lower intensity. (c) Spectrum of Trip 1 from wash 3. The mass is no longer observed. (d) Spectrum of the lysate of cells incubated with Trip 1, showing an m/z corresponding to the desired mass. (e) Cell lysate in which no triptycene was added, demonstrating that the mass is not observed in normal cell lysates. Column 2: Spectra for Trip 2. (f) Spectrum of Trip 2 from wash 1. A m/z=723.781 was observed for the triptycene. (g) Spectrum of Trip 2 from wash 2. The mass of Trip 2 is no longer present. (h) Spectrum of Trip 2 from wash 3. (i) Spectrum of the lysate of cells incubated with Trip 2, showing an m/z corresponding to the desired mass. (j) Cell lysate in which no triptycene was added, demonstrating that the mass is not observed in normal cell lysates. Column 3: Spectra for Trip 3. (k) Spectrum of Trip 3 from wash 1. A m/z=639.550 was observed for the triptycene. (1) Spectrum of Trip 3 from wash 2. The mass of Trip 3 is still present at a lower intensity. (m) Spectrum of Trip 3 from wash 3. The mass is no longer observed. (n) Spectrum of the lysate of cells incubated with Trip 3. The desired mass is not observed demonstrating that Trip 3 was not taken up into the cells. (o) Cell lysate in which no triptycene was added, demonstrating that the mass is not observed in normal cell lysates.

FIG. 42 is Example 3 depiction of (a) Schematic of gel shift assay. (b) The folded TNR 3WJ was incubated with different concentrations of an inhibitor strand (I10) complementary to the 5′-end, resulting in formation of TNR-I10.

FIG. 43 is the Example 3 depiction of (a) Schematic of gel shift assay. The folded TNR 3WJ was incubated with an inhibitor strand complementary to the 5′-end, opening the junction structure (TNR-I10). Addition of triptycene results in reformation of the junction (TNR-Trip). (b) Structures of triptycene derivatives. (c) Gel shift assay where TNR-I10 was incubated with triptycene derivatives at a constant concentration. (d) A plot of the difference in band intensities of TNR and TNR-I10. Bars below zero in the plot indicated an increased amount of complex relative to 3WJ. (e) Gel shift assay in the presence and absence of Trip 3 and Trip 4. Samples contained 0.5 μM TNR alone (where minus sign is indicated) or 0.5 μM TNR and 1.5 μM I10 (where a plus sign is indicated). Increasing concentrations of Trip 3 were added (lane 3, 0 μM; lane 4, 0.01 μM; lane 5, 0.10 μM; lane 6, 0.50 μM; lane 7, 1.0 μM; lane 8, 5.0 μM) and Trip 4 (lane 11, 0 μM; lane 12, 0.01 μM; lane 13, 0.10 μM; lane 14, 0.50 μM; lane 15, 1.0 μM; lane 16, 5.0 μM; lane 17, 10.0 μM). Lanes 1 and 9 are loaded with a 25 base pair DNA ladder in which the band present corresponds to 25 bases. Free TNR junction and TNR-I10 complex are indicated. Non-denaturing polyacrylamide gel ran in 1×TBE buffer at 4° C.

FIG. 44 is the Example 3 depiction of Fluorescence-quenching assay and circular dichroism (CD). (a) TNR 3WJ was labeled with a fluorophore and quencher. When folded, low fluorescence is observed. Addition of inhibitor I10, opens the junction resulting in an increase in fluorescence (TNR*-I10). Addition of triptycene, reforms the junction, resulting in quenching of fluorescence. (b) Titration of I10 to the folded junction results in an increase in fluorescence. Fluorescence assay was conducted in 50 mM sodium phosphate buffer at pH 7.2. (c) Titration of Trip 3 and Trip 4 to TNR*-I10 results in a decrease in fluorescence. (d) Temperature-dependent circular dichroism of the TNR junction. Temperature-dependent CD in the presence of Trip 3 (e) and Trip 4 (f). Circular dichroism measurements were conducted in 50 mM sodium phosphate buffer at pH 7.2.

FIG. 45 depicts a synthetic scheme of the present invention.

FIG. 46 depicts a synthetic scheme of the present invention.

FIG. 47 shows different triptycene derivatives.

FIG. 48 shows different triptycene derivatives.

FIG. 49 shows cytotoxicity and cell uptake studies in Example 1 using human ovarian carcinoma cell lines. (a) Percent viability of A2780, a cisplatin sensitive ovarian cancer cell line, and A2780cis, a cisplatin-sensitive ovarian cancer cell line, in the presence of triptycenes 1-3 or cisplatin. Viability is shown at a final concentration of 50 μM for each compound. All experiments were conducted in duplicate and the asterisk indicates zero viability. (b) Cell uptake studies using MALDI-MS for Trip 1-3 in A2780 cells. Asterisk=no detectible compound.

FIG. 50 shows reaction conditions for amide bond formation at the linker position in Example 4.

FIG. 51 shows circular dichroism of model system RNA at different concentrations of Trip 1 (a,b) or Trip 2 (c,d) according to Example 2. Note that the Trip designation of compounds may be different compounds in different Examples.

FIG. 52 shows initial screening of triptycenes using 632-GFP fusion assay in Example 2. (a) Structures of triptycenes tested. (b) Relative fluorescence intensity of GFP control and 632-GFP fusion at 30° C. and 42° C. in the presence of 25 μM triptycenes.

FIG. 53 shows relative fluorescence intensity of GFP control and 632-GFP fusion at 30° C. and 42° C. at varying concentrations of Trip 1 (a) or Trip 2 (b) according to Example 2.

FIG. 54 shows bacterial growth at 37° C. in the absence or presence of Trip 1 or Trip 2 at different concentrations according to Example 2.

FIG. 55 shows mRNA expression levels determine by qRT-PCR in the absence or presence of Trip 1 or Trip 2 at 12.5 μM or 25 μM according to Example 2. (a) Ct ratio of rpoH/rrsG (b) Ct ratio of rpoH/arcA (c) Normalized rpoH expression level against rrsG (d) Normalized rpoH expression level against arcA.

FIG. 56 shows an approach towards synthesis of 9-substituted triptycene based scaffold which can be used as a building block for solid-phase peptide synthesis and rapid diversification according to Example 4.

FIG. 57 shows a scheme of a strategy for triptycene solid-phase diversification and retrosynthesis of key building block A^{a} of Example 4. ^{a}FGI=functional group interconversion; ox.=oxidation; red.=reduction; DA=Diels-Alder reaction.

FIG. 58 shows a scheme of an approach toward the synthesis of 9-substituted trifunctionalized triptycenes 6a-c and X-ray crystal structure of 5a in Example 4.

FIG. 59 shows a scheme of composition of 6a-c from the nitration of Compounds 4, 7, and 8 in Example 4.

FIG. 60 shows a scheme of synthesis of SPPS precursor 12 and loading on 2-chlorotrityl chloride resin in Example 4.

FIG. 61 shows a scheme according to Example 4: (a) solid-phase peptide synthesis of 9-substituted triptycene on 2-chlorotrityl chloride resin; (b) cleavage from the resin to generate triptycene derivatives 17-19.

FIG. 62 shows according to Example 4: (a) graphical representation of the fluorescence-quenching 3WJ assay. (b) dissociation constants of triptycenes 17-20.

FIG. 63 shows chromatogram of crude nitration mixture from compound 4 in Example 4.

FIG. 64 shows chromatogram of crude nitration mixture from compound 7 in Example 4.

FIG. 65 shows chromatogram of crude nitration mixture from compound 8 in Example 4.

FIG. 66 shows merged chromatogram of crude nitration mixtures from compound 4, 7, and 8 in Example 4.

FIG. 67 shows chromatogram of analytical HPLC of compound 12 in Example 4.

FIG. 68 shows chromatogram of analytical HPLC of compound 17 in Example 4.

FIG. 69 shows chromatogram of analytical HPLC of compound 18 in Example 4.

FIG. 70 shows chromatogram of analytical HPLC of compound 19 in Example 4.

FIG. 71 shows MALDI-MS data of compound 12 in Example 4. Calculated for C₆₉H₅₂N₄NaO₉⁺ [M+Na]⁺ 1103.363, found 1103.873; C₆₉H₅₂KN₄O₉⁺ [M+K]⁺ 1119.337, found 1119.863; C₆₉H₅₁N₄Na₂O₉⁺ [M−H+2Na]⁺ 1125.345, found 1125.873.

FIG. 72 shows MALDI-MS data of compound 17 in Example 4. Calculated for C₄₂H₄₄N₁₃O₆⁺ [M+H]⁺ 826.353, found 826.690; C₄₂H₄₃N₁₃NaO₆⁺ [M+Na]⁺ 848.335, found 848.679; C₄₂H₄₂N₁₃Na₂O₆⁺ [M−H+2Na]⁺ 870.317, found 870.670.

FIG. 73 shows MALDI-MS data of compound 18 in Example 4. Calculated for C₆₀H₈₀N₁₉O₉⁺ [M-41]⁺ 1210.638, found 1211.290; C₆₀H₇₉N₁₉NaO₉⁺ [M+Na]⁺ 1232.620, found 1233.284; C₆₀H₇₈N₁₉Na₂O₉⁺ [M−H+2Na]⁺ 1254.602, found 1255.278.

FIG. 74 shows MALDI-MS data of compound 19 in Example 4. Calculated for C₇₂H₉₈H₂₅O₁₅⁺ [M+H]⁺ 1552.767, found 1553.231; C₇₂H₉₇N₂₅NaO₁₅⁺ [M+Na]⁺ 1574.749, found 1575.218; C₇₂H₉₆N₂₅Na₂O₁₅⁺ [M−H+2Na]⁺ 1596.731, found 1597.206.

FIG. 75 shows fluorescence-quenching assay for triptycenes 17 (A), 18 (B), 19 (C), and 20* (D) in Example 4. *Triptycene 20 is an analogue of triptycene 17 lacking a linker at the C9 position.

FIG. 76 shows a rapid and efficient approach towards synthesis of bridgehead-substituted triptycenes according to Example 5.

FIG. 77 shows (a) Schematic of triptycene bound to a three-way junction and a key triptycene building block for diversification by solid-phase synthesis according to Example 5. (b) Improvement of the synthesis of triptycene intermediates in this work (Example 5) compared with previous work (Example 4).

FIG. 78 shows a scheme of synthesis of bridgehead-substituted triptycene 5a-d in Example 5.

FIG. 79 shows a scheme of solid-phase synthesis of orthogonally protected building block 7 and fluorescence-quenching experiment of triptycene peptides in Example 5.

FIG. 80 shows crude HPLC chromatograms after cleavage from 2-chlorotrityl chloride resin in Example 5.

FIG. 81 shows HPLC chromatograms of purified compounds 8-12 in Example 5.

FIG. 82 shows fluorescence-quenching experiment plots in Example 5. Displacement of I10 from TNR 3WJ by Trip-(Gly-Lys)₃ (a), Trip-(Gly-His)₃ (b), Trip-(His-Lys-His)₃ (c), Trip-(His-Lys-Lys)₃ (d), Trip-(His-Lys-Asn)₃ (e). An overlay of all plot is shown in (f).

FIG. 83 shows gel shift assay in the presence of triptycenes in Example 5. TNR 3WJ was incubated with I10 followed by titration of triptycene derivatives, Gly-Lys (a), Gly-His (b), His-Lys-His (c), His-Lys-Lys (d), or His-Lys-Asn (e).

FIG. 84 shows the crystal data and structure refinement for 5c of Example 4.

FIG. 85 shows the calculated and observed triptycene masses of Example 5.

FIG. 86 shows crystal data and structure refinement for 5d of Example 5.

### DETAILED DESCRIPTION OF THE INVENTION

This application expressly incorporates by reference in the entirety Barros et al., Angew Chem Int Ed Engl., 2014(53):13746, published on Sep. 24, 2014; Barros et al., Angew. Chem. Int. Ed., 2016(55):8258, published on May 30, 2016; Barros et al., Chemical Science, 2015(6):4752, published on Jun. 10, 2015; Yoon et al., Organic Letters, 2016(18):1096, published on Feb. 17, 2016; and Barros et al., Organic Letters, 2016(18):2423, published on May 12, 2016.

### I. Overview

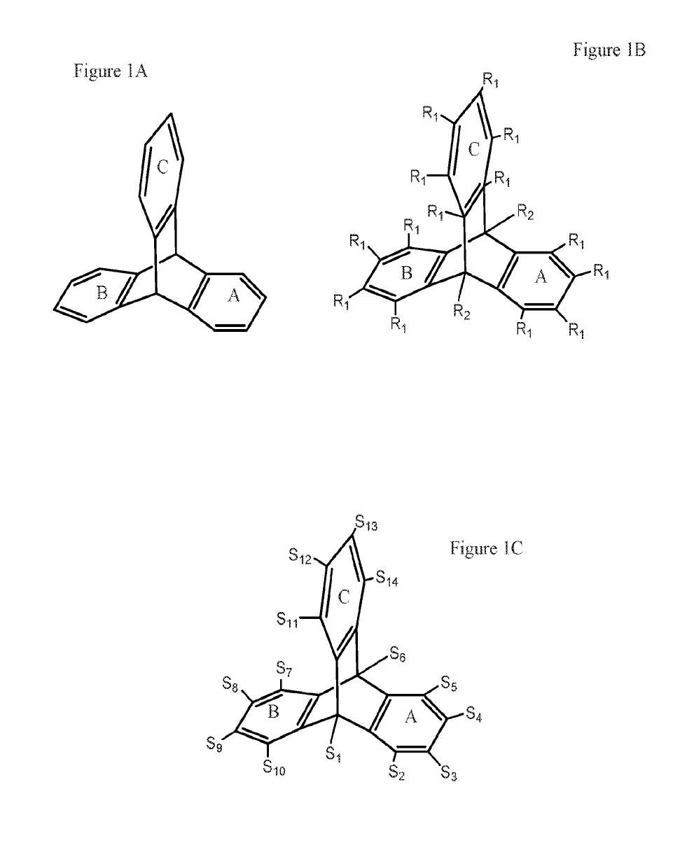

The present invention is directed to the recognition that triptycene, shown in FIG. 1, possesses a threefold symmetric architecture with dimensions similar to those of the central helical interface of a perfectly base-paired nucleic acid three-way junction (TWJ). Accordingly, the invention provides a new class of structure-specific nucleic acid junction stabilizers based on this TC scaffold.

As outlined above, TWJs play important roles in biological processes. TWJs are found as transient intermediates during replication, recombination and DNA damage repair. Junctions are also present in several viral genomes, such as HIV-1, HCV and adeno-associated virus in addition to playing key roles in viral assembly. TWJs also occur in trinucleotide repeat expansions found in unstable genomic DNA associated with neurodegenerative diseases. In addition, TWJs are important in a number of bacterial processes, including the heat shock response (HSF).

Accordingly, the ability to stabilize such junctions, e.g. to “lock” a structure into a TWJ, can allow for the inhibition of certain biological processes that result in the treatment or amelioration of disease, including cancer and pathogen infections. For example, locking specific TWJs in place prevents the induction of the heat shock response in bacteria, thus leading to a new class of antibiotics. Similarly, the triptycene derivatives (TCDs) of the invention can be used to halt DNA replication, similar to the metal helicates such as cisplatin, and thus used as cytotoxic (including chemotherapeutic) agents. Furthermore, trinucleotide repeat nucleic acid sequences are associated with a large number (>30) of inherited human muscular and neurological diseases. The trinucleotide repeat tract length is dynamic and often correlates with disease severity, where short stable tracts are commonplace in the non-affected population. Longer unstable triplet repeat tracts are more prone to expansion as opposed to contraction, in addition to being predisposed to generational transmission. Trincleotide repeat repair outcomes are also affected by structural features present in slipped sequences, where the structure may determine which proteins are recruited for repair. Stabilization of a particular structure could lead to increased repair of these slipped-out junction. Addition of ligands that bind to these junctions can affect repair outcomes as well as recruitment of proteins.

It is important to note that the three strands of nucleic acid that make up a TWJ can come from one strand that folds into a junction, two strands that assemble to form a junction, or three separate strands that assemble to form a junction, all of which have important biological ramifications, and depend on whether the junctions form naturally or not. That is, in some cases, the TWJ occurs naturally, such as with the rho temperature system, and TCDs are added to disrupt the junction or lock it into place as needed. Alternatively, the TWJ can be induced to form using one or more exogeneous nucleic acid strands in combination with a TCD.

For example the oligonucleotides comprising a junction could come from multiple natural sources and unnatural sources. An example is using a triptycene to form a junction between one oligonucleotide sequence from a human source, one from a viral source, and one from a unnatural source. This is a heterotrimeric junction that binds the TCD. It should be noted that a junction of this kind may not form in the absence of the TCD but forms in the presence of the TCD or is stabilized to a greater extent.

In this way, TCDs are used for direct therapeutic benefit, augmentation of oligonucleotide therapeutics, augmentation of endogenous oligonucleotides, induction of cryptic junctions, allosteric modulation of junctions, use in oligonucleotide diagnostics, use in oligonucleotide sensors, in PCR applications, or in any other capacity where formation, modulation, induction, or perturbation of a junction exerts a desired effect. For example, a micro RNA that anneals to an endogenous oligonucleotide sequence to form a junction, the introduction of a triptycene simply binds this complex and potentiates the effect in come way. In this embodiment, the triptycene is not causing the direct effect but rather augmenting or enhancing the effect that is already there.

Thus, TCDs can be used to “lock” an existing, naturally occuring or endogeneous structure in a sequence specific manner.

Alternatively, TCDs can be used in conjunction with other introduced (or endogeneous) nucleic acids to both form and lock a structure in a sequence specific manner. That is, exogeneous TWJs can be introduced using the administration of nucleic acid strands that will participate in a TWJ in the presence of a TCD either in an intermolecular or intramolecular configuration. As will be appreciated by those in the art, the junction may not pre-exist prior to interaction with the TCD. In some cases, the TCD induces formation of a junction that was not present prior to interaction with a triptycene or other small molecule.

Furthermore, the inhibitor displacement assay outlined herein shows that the equilibrium between one strand can be shifted to a new structure comprised of an intramolecular junction with one strand. This is not limited to this case and can be any structure of any number of initial strands that upon interaction with a triptycene small molecule converts into a new junction structure comprised of any number of strands. The new structure can contain oligonucleotide strands from the initial structure in combination with new strands from both natural and synthetic sources to form a junction structure in complex with a shape selective binding molecule such as triptycene.

As alluded to herein, one or more of the strands forming the junction could be DNA, RNA, nucleic acid analogs or hybrids of both DNA and RNA from natural or synthetic sources. Additionally, the junctions could be formed by alternative synthetic mimicking oligonucleotide structures such as PNA, LNA, or other oligomeric nucleic acid mimicking and targeting technologies. Triptycenes could be used to target hybrid junctions created or formed from mixed strands in an intermolecular or intramolecular sense containing DNA, RNA, PNA, LNA, or any other oligonucleotide recognizing technology.

For example, if one strand forms a junction, the terminal loops can be any size, an example might be annealing regions separated by many base pairs that fold to form a junction. The synthetic oligonucleotides could be of therapeutic relevance such as siRNA or medicinal aptamers.

Furthermore, the invention provides methods of generating new TCDs, including libraries, both in solution as well as immobilized on solid supports. Bridgehead (S1, S6) or non-bridgehead positions (S2-S5, S7 to S14) can be used as possible attachment sites for attachment to a solid support (FIG. 15). These libraries include triptycene derivatized with traditional organic substituents as well as amino acid side chains, peptides and nucleic acid components, including polynucleotides. In addition, the invention provides methods of screening individual TCDs for biological activity, as well as methods of screening TCD libraries against TWJs with different sequences, such that sequence specific stabilization/inhibition occurs.

Accordingly, the present invention is directed to compositions and methods relying on TCDs.

### II. Triptycene Derivatives

The present invention provides TCDs, as compositions and for use in methods. Triptycene, shown in FIG. 1, is insoluble in aqueous solution and thus must be derivatized with solubilization substituents for use in biological applications; in addition, the substituents are chosen to increase activity and/or specificity and selectivity. That is, R groups are added to a triptycene to allow the TCD to distinguish between TWJs of different sequences. FIG. 1B shows the 14 R1 “ring substituent” positions and the 2 R2 “bridgehead” substituents, any and all of which may be modified to produce TCDs.

In some embodiments, the invention provides water soluble TCDs. The term “water-soluble” refers to moieties that have some detectable degree of solubility in water. Methods to detect and/or quantify water solubility are well known in the art. Thus, in general, at least one “R” position of FIG. 1, whether R1 or R2 positions (or, using FIG. 1C nomenclature, any substitutent position S), contain a solubility R group, with some embodiments (depending on the length and solubility of the R group) containing more than one solubility R group.

Substituent “R” groups fall into several categories, including traditional organic compounds such as alkyl groups (including heteroalkyl and substituted alkyl groups), aryl groups (including heteroaryl and substituted aryl groups) as described below, as well as amino acid side chains and analogs, as well as nucleic acids and analogs.

In some embodiments, the R groups are solubility conferring R groups. Suitable R groups include, but are not limited to, hydrogen, alkyl, alcohol, aromatic, amino, amido, nitro, ethers, esters, aldehydes, sulfonyl, silicon moieties, halogens, sulfur containing moieties, phosphorus containing moieties, and ethylene glycols. In the structures depicted herein, R is hydrogen when the position is unsubstituted. It should be noted that some positions may allow two substitution groups, R and R′, in which case the R and R′ groups may be either the same or different.

By “alkyl group” or grammatical equivalents herein is meant a straight or branched chain alkyl group, with straight chain alkyl groups being preferred. If branched, it may be branched at one or more positions, and unless specified, at any position. The alkyl group may range from about 1 to about 30 carbon atoms (C1-C30), with a preferred embodiment utilizing from about 1 to about 20 carbon atoms (C1-C20), with about C1 through about Cl2 to about C15 being preferred, and C1 to C5 being particularly preferred, although in some embodiments the alkyl group may be much larger. Also included within the definition of an alkyl group are cycloalkyl groups such as C5 and C6 rings, and heterocyclic rings with nitrogen, oxygen, sulfur or phosphorus. Alkyl also includes heteroalkyl, with heteroatoms of sulfur, oxygen, nitrogen, and silicone being preferred. Alkyl includes substituted alkyl groups. By “substituted alkyl group” herein is meant an alkyl group further comprising one or more substitution moieties “R”, as defined above. One preferred linkage of an alkyl group to the TC molecule is using amido groups.

By “amino groups” or grammatical equivalents herein is meant —NH₂, —NHR and —NR₂ groups, with R being as defined herein.

By “nitro group” herein is meant an —NO₂ group.

By “sulfur containing moieties” herein is meant compounds containing sulfur atoms, including but not limited to, thia-, thio- and sulfo-compounds, thiols (—SH and —SR), and sulfides (—RSR—). By “phosphorus containing moieties” herein is meant compounds containing phosphorus, including, but not limited to, phosphines and phosphates. By “silicon containing moieties” herein is meant compounds containing silicon.

By “ether” herein is meant an —O—R group. Preferred ethers include alkoxy groups, with —O—(CH₂)₂CH₃ and —O—(CH₂)₄CH₃ being preferred.

By “ester” herein is meant a —COOR group.

By “halogen” herein is meant bromine, iodine, chlorine, or fluorine. Preferred substituted alkyls are partially or fully halogenated alkyls such as CF₃, etc.

By “aldehyde” herein is meant —RCHO groups.

By “alcohol” herein is meant —OH groups, and alkyl alcohols —ROH.

By “amido” herein is meant —RCONH— or RCONR— groups.

By “ethylene glycol” or “(poly)ethylene glycol” (PEG) herein is meant a —(O-CH₂—CH₂)ₙ— (heteroalkyl) group, although each carbon atom of the ethylene group may also be singly or doubly substituted, e.g. —(O—CR₂—CR₂)ₙ—, with R as described herein. Ethylene glycol derivatives with other heteroatoms in place of oxygen (i.e. —(N—CH₂—CH₂)ₙ— or —(S—CH₂—CH₂)ₙ—, or with substitution groups) are also preferred.

Preferred substitution groups include, but are not limited to, methyl, ethyl, propyl, alkoxy groups such as —O—(CH₂)₂CH₃ and —O—(CH₂)₄CH₃ and ethylene glycol and derivatives thereof.

Preferred aromatic groups include, but are not limited to, phenyl, naphthyl, naphthalene, anthracene, phenanthroline, pyrole, pyridine, thiophene, porphyrins, and substituted derivatives of each of these, included fused ring derivatives.

In some embodiments, the “R” alkyl and/or aryl groups (including heteroalkyl and heteroaryl) can be further substituted with additional R groups, such as an amino substituted phenyl group or a hydroxy phenyl group.

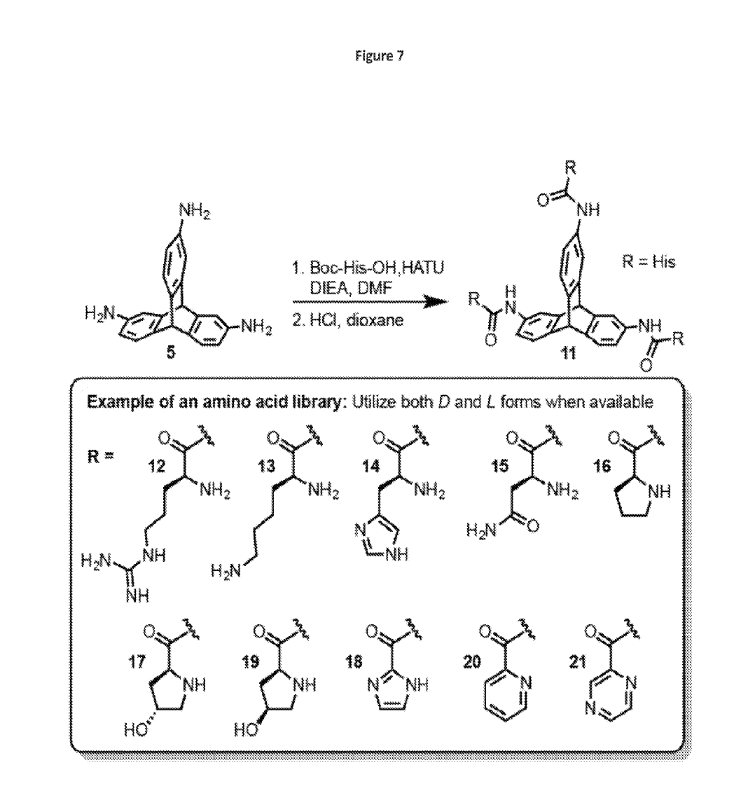

In some embodiments, the R groups are proteinaceous in nature, generally including proteins, which includes peptides and amino acid side chains and side chain analogs, including both monomers (e.g. a single amino acid side chain or analog) as well as multimers (e.g. peptides and peptide analogs, including dimers (two amino acids), trimers (three), tetramers, etc.). By “proteins” or grammatical equivalents herein is meant proteins, oligopeptides and peptides, derivatives and analogs, including proteins containing non-naturally occurring amino acids and amino acid analogs, and peptidomimetic structures. The side chains may be in either the (R) or the (S) configuration. In a preferred embodiment, the amino acids are in the (S) or L-configuration. Some suitable amino acid side chains and analogs are shown in FIG. 7.

In some embodiments, the R groups are nucleic acids and/or nucleic acid analogs. (For clarity, it should be noted that there are nucleic acid R groups, sometimes referred to herein as “R group nucleic acid radicals”, in addition to the nucleic acid substrates and inhibitors outlined herein, and this definition applies to both). The nucleic acids can be single stranded or double stranded, as specified, or contain portions of both double stranded or single stranded sequence. The nucleic acid can be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. A preferred embodiment utilizes isocytosine and isoguanine in nucleic acids designed to be complementary to other probes, for example when the nucleic acids are part of the substrate or inhibitors as discussed herein, as this reduces non-specific hybridization, as is generally described in U.S. Pat. No. 5,681,702. As used herein, the term “nucleoside” includes nucleotides as well as nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

As for the amino acid R groups, nucleic acid analogs include both monomers (e.g. a single nucleotide or nucleoside, or analog) as well as multimers (e.g. oligonucleotides and analogs). “Oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989)), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with bicyclic structures including locked nucleic acids, (Koshkin et al., J. Am. Chem. Soc. 120:13252-3 (1998)); positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference.

In some embodiments, peptide nucleic acids (PNA) find use, which includes peptide nucleic acid analogs. These backbones are substantially non-ionic under neutral conditions, in contrast to the highly charged phosphodiester backbone of naturally occurring nucleic acids. This results in several advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tm for an internal mismatch. With the non-ionic PNA backbone, the drop is closer to 7-9° C. Similarly, due to their non-ionic nature, hybridization of the bases attached to these backbones is relatively insensitive to salt concentration. Furthermore, due to their synthetic nature, PNAs are not digested by native enzymes, making them very stable in in vivo applications.

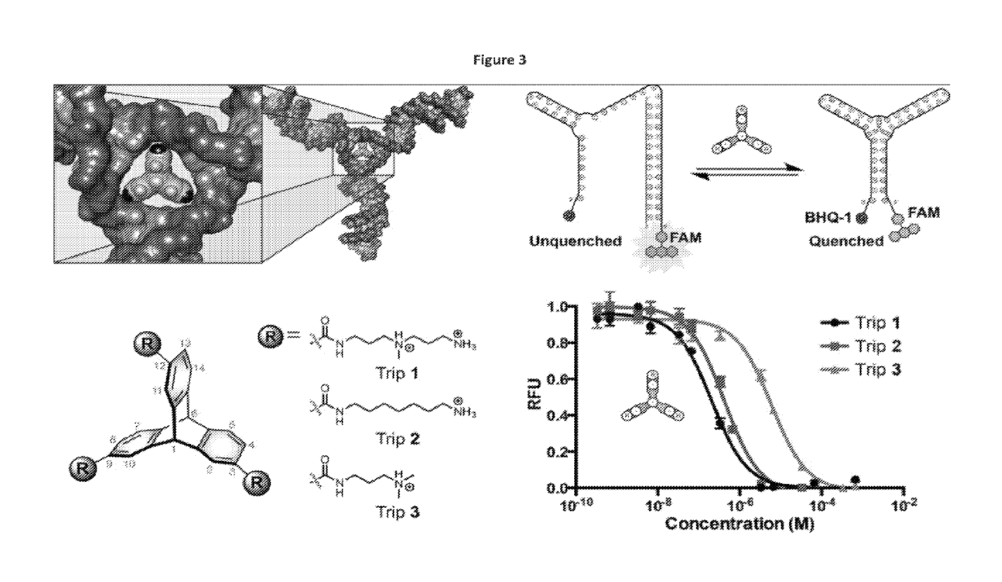

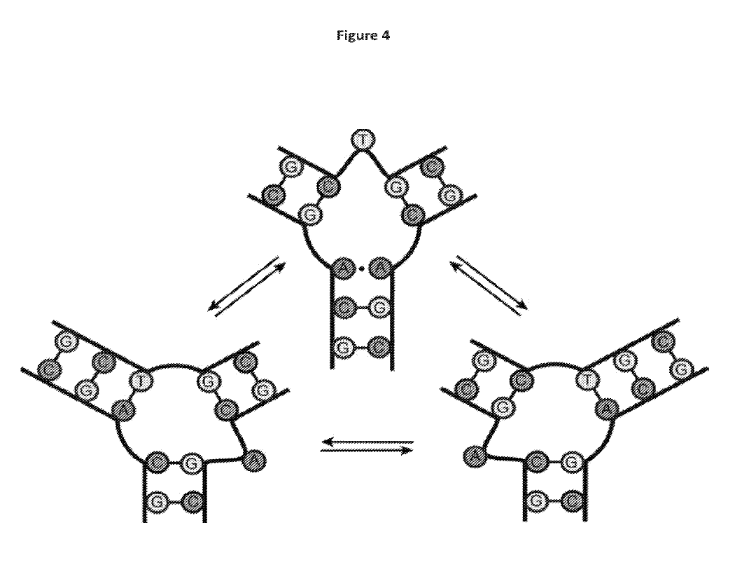

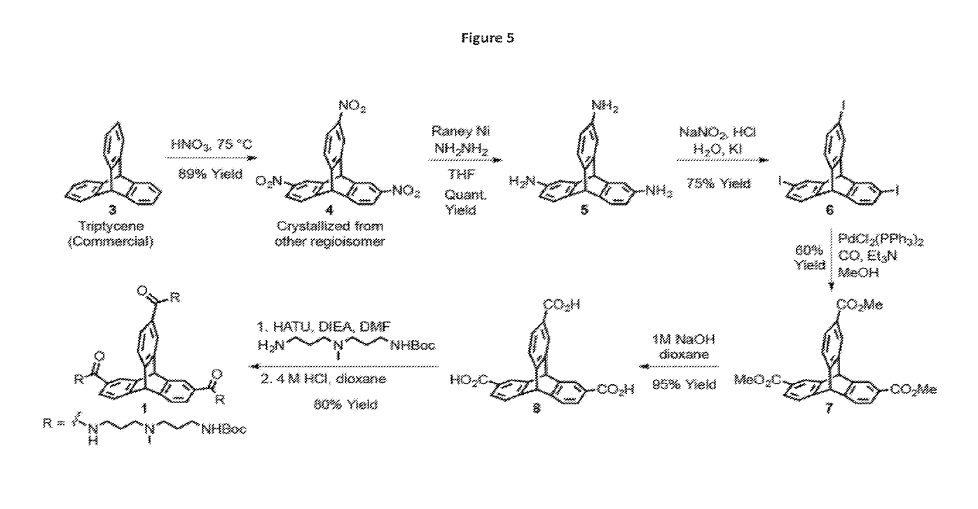

The number and orientation of the R groups (both R1 and R2 in FIG. 1B) can vary, as will be appreciated by those in the art, to create TCDs with from 1 to 14 R groups, either being the same, or different, or combinations thereof. For example, FIG. 3 depicts three different tri-substituted TCDs, each with the same R group on the same position on each of the three rings (e.g. the same substitution pattern). TCDs with one R group off of just one ring (A), or 2 R groups (A or B), as well as all three rings (A, B and C) can also be made. In addition, these substitutions may be symmetrical, as shown in FIG. 3, wherein each identical R group is on the same position on the ring (e.g. S3, S9 and S12, using the numbering of FIG. 1C; e.g. the same substitution pattern) or different. In embodiments with more than one (different) R groups are used, these may be attached at the same position on each ring, or different positions. In this way, sequence specificity can be built into the TCD.

The inhibitor displacement assay in FIG. 3 is an example where the equilibrium between two strands can be shifted to a new structure comprised of an intramolecular junction with one strand. It should be noted that the invention is not limited to this case and can be any structure of any number of initial strands that upon interaction with a triptycene small molecule converts into a new junction structure comprised of any number of strands. In some embodiments, the new structure contains oligonucleotide strands from the initial structure in combination with new strands from both natural and synthetic sources to form a junction structure in complex with a shape selective binding molecule such as triptycene.

In some embodiments, substitutions at the R2 positions are done for immobilization of the TCD onto a solid support. This may be done for several reasons, including, but not limited to, for chemical synthesis of additional derivatives as well as for immobilization of TCDs for screening against a variety of TWJs, particularly for use in identifying TCDs that are sequence specific, e.g. will bind one particular TWJ preferentially over those of different sequences (although as outlined herein, that may not be required in all applications, particularly in chemotherapeutic applications). In some embodiments, the R2 positions are used for additional substituent substitution for the purposes of gaining sequence selectivity and specificity.

In some cases, the TCD may have an inherent fluorescence built in by addition of R groups that fluoresce (although will generally only be done when other R groups in the TCD confer significant solubility, as many fluorophores are also quite hydrophobic and water insoluble). This may be done to measure TWJ binding directly, if the fluorescent R group changes it's fluorescent profile in the hydrophobic pocket of the junction; that is, a change in fluorescence of the TCD when in solution versus bound in the junction can be used to assay binding.

Arrays of TCDs

As discussed herein, the present invention provides solid supports comprising arrays of TCD generally at least a first substrate with a surface comprising a plurality of assay locations. By “array” herein is meant a plurality of TCDs in an array format; the size of the array will depend on the composition and end use of the array. Arrays containing from about 2 different TCDs to many millions can be made, with many embodiments using microtiter plate arrays.

By “substrate” or “solid support” or other grammatical equivalents herein is meant any material that can be modified to contain discrete individual sites appropriate for the attachment, association or synthesis of TCDs and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, plastics, optical fiber bundles, and a variety of other polymers. In general, the substrates allow optical detection and do not themselves appreciably fluorescese.

Generally the substrate is flat (planar), although as will be appreciated by those in the art, other configurations of substrates may be used as well; for example, three dimensional configurations can be used. Preferred substrates include flat planar substrates such as glass, polystyrene and other plastics and acrylics, with microtiter plate formats finding use in many embodiments. In some embodiments, silicon wafer substrates can be used.

The solid support comprises a surface comprising a plurality of assay locations, i.e. the location where the TCD will be placed or synthesized. The assay locations are generally physically separated from each other, for example as assay wells in a microtiter plate, although other configurations (hydrophobicity/hydrophilicity, etc.) can be used to separate the assay locations.

The sites may be a pattern, i.e. a regular design or configuration, or randomly distributed. A preferred embodiment utilizes a regular pattern of sites such that the sites may be addressed in the X-Y coordinate plane. “Pattern” in this sense includes a repeating unit cell.

In some embodiments, the surface of the substrate is modified to contain modified sites, particularly chemically modified sites, that can be used to attach, either covalently or non-covalently, the TCDs of the invention to the discrete sites or locations on the substrate. “Chemically modified sites” in this context includes, but is not limited to, the addition of a pattern of chemical functional groups including amino groups, carboxy groups, oxo groups and thiol groups, that can be used to covalently attach TCDs, which generally also contain corresponding reactive functional groups.

In some embodiments, the TCDs may be synthesized or attached to beads (which can be magnetic, in some cases), and then put down on a second support in an array pattern; for example; the addition of a pattern of adhesive that can be used to bind the microspheres with TCDs (either by prior chemical functionalization for the addition of the adhesive or direct addition of the adhesive); the addition of a pattern of charged groups (similar to the chemical functionalities) for the electrostatic attachment of the microspheres, i.e. when the microspheres comprise charged groups opposite to the sites; the addition of a pattern of chemical functional groups that renders the sites differentially hydrophobic or hydrophilic, such that the addition of similarly hydrophobic or hydrophilic microspheres under suitable experimental conditions will result in association of the microspheres to the sites on the basis of hydroaffinity. For example, the use of hydrophobic sites with hydrophobic beads, in an aqueous system, drives the association of the beads preferentially onto the sites.

In some embodiments, libraries are made of different TCDs for screening. “Library” in this context means a plurality of different TCD compounds, from 2 to millions, depending on the synthetic options.

### III. Target Nucleic Acid Three Way Junctions/Target Substrates

The TCDs of the invention are used to stabilize TWJs, sometimes also referred to as nucleic acid target substrates, as the structure that is acted upon by the TWJ to stabilize it thermodynamically, thus preventing normal biological processes from occurring.

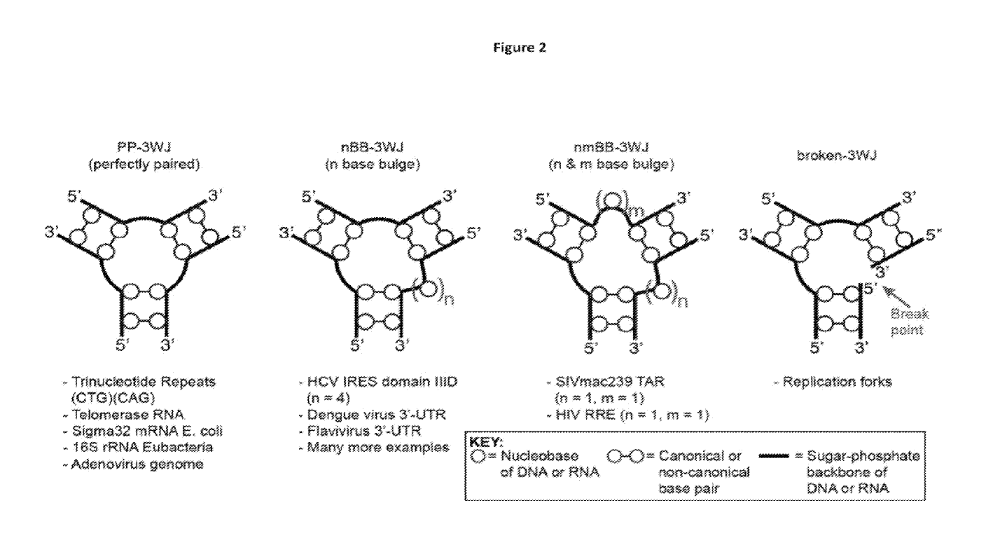

In general, preferred TWJs are those that are found in such normal biological processes, including disease processes such as pathogen infection and replication as well as cancer. As shown in FIG. 2, there are specific types of TWJs, including perfectly paired junctions, as well as base-bulged and broken junctions, that play roles in various biological processes. In some embodiments, one strand folds into and form a junction where the terminal loops can be any size, for example, annealing regions separated by many base pairs that fold to form a junction. In some embodiments, two strands assemble to form a junction. In some embodiments, three separate strands assemble to form a junction.

All strands forming the junction can be DNA, RNA, or hybrids of both DNA and RNA from natural or synthetic sources. The DNA and RNA strands comprising the junction can be a combination of natural and synthetic oligonucleotides. The synthetic oligonucleotides can be of therapeutic relevance such as siRNA or medicinal aptamers. In some embodiments, the oligonucleotides comprising a junction come from multiple natural sources and unnatural sources, as shown in an example using a triptycene to form a junction between one oligonucleotide sequence from a human source, one from a viral source, and one from an unnatural source. This would be a heterotrimeric junction binding a small molecule such as a triptycene. In some embodiments, the junctions are formed by alternative synthetic mimicking oligonucleotide structures such as peptide nucleic acid (PNA), locked nucleic acid (LNA), or other oligomeric nucleic acid mimicking and targeting technologies.

In some embodiments, a junction may not pre-exist prior to interaction with a triptycene or its derivative or other small molecule. A junction may form or be stabilized to a greater extent in the presense of a small molecule, such as triptycene or or its derivative.

The TWJ substrates of the invention take on a number of formats, and can be a variety of lengths and sequences, and can be naturally occurring nucleic acids (e.g. the sequences are both naturally occurring as well as made of standard nucleotides). In some cases, particularly for screening applications, the nucleic acid TWJs can comprise nucleic acid analogs as needed, as described above.

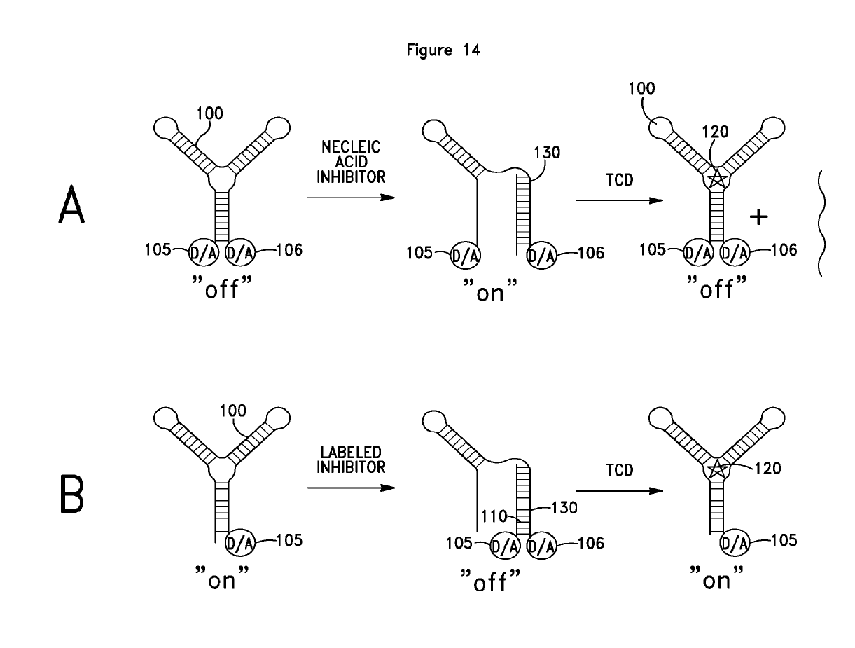

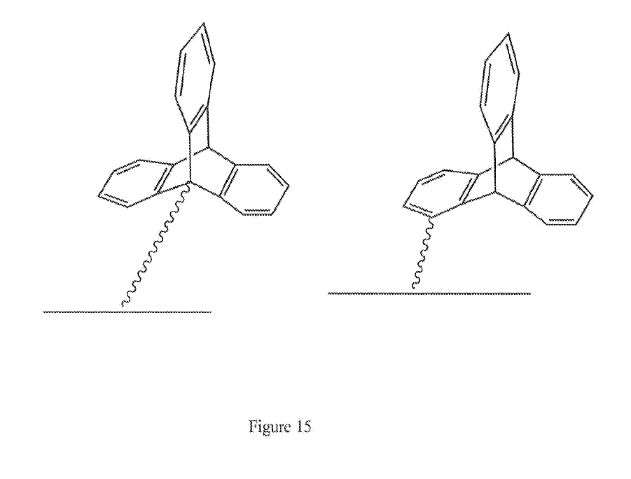

As shown in FIG. 14, the TWJ substrates are generally labeled with either one or two FRET labels, depending on the assay format and the inhibitor labeling (or lack thereof). FRET donor/acceptor dye pairs are well known in the art, including Black Hole Quenchers®, hereby incorporated by reference for the disclosure of FRET pairs.

Triptycenes can be used to target hybrid junctions created or formed from mixed strands in an intermolecular or intramolecular sense containing DNA, RNA, PNA, LNA, or any other oligonucleotide recognizing technology. Triptycenes can also be used for direct therapeutic benefit, augmentation of oligonucleotide therapeutics, augmentation of endogenous oligonucleotides, induction of cryptic junctions, allosteric modulation of junctions, use in oligonucleotide diagnostics, use in oligonucleotide sensors, in PCR applications, or in any other capacity where formation, modulation, induction, or perturbation of a junction might exert a desired effect. For example, a micro RNA is annealed to an endogenous oligonucleotide sequence to form a complex and a triptycene or its derivative binds this complex and augment this effect.

### IV. Assay Inhibitors

The assays of the invention generally include an inhibitor, which, again, with reference to FIG. 14 can either be labeled or unlabeled with a FRET donor or acceptor, depending on the format desired. Inhibitors are generally used herein to indirectly detect TCD binding to a substrate since the size of the TCD is so small compared to the size of the TWJ substrate. In addition, inhibitors are designed in length such that in the presence of the inhibitor, the TWJ substrate favors the binding of the inhibitor over the formation of the junction in normal physiological environments. This will be somewhat specific to the TWJ being investigated, but generally the inhibitor will be from 10 to 25 nucleotides long. In addition, the inhibitor should base pair with at least one or two of the immediate junction nucleotides, to break up the structure.

### V. Screening Assays

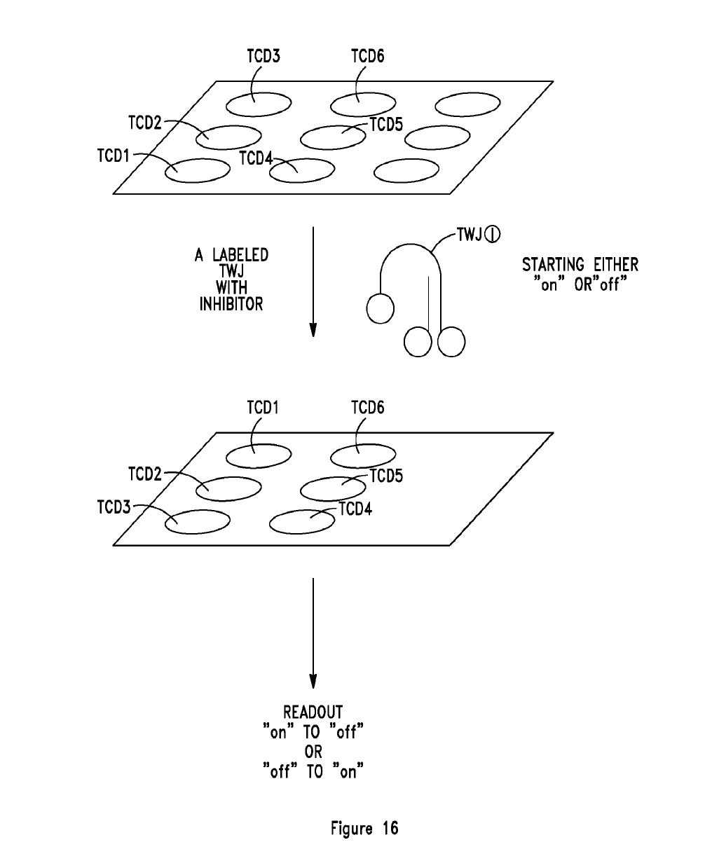

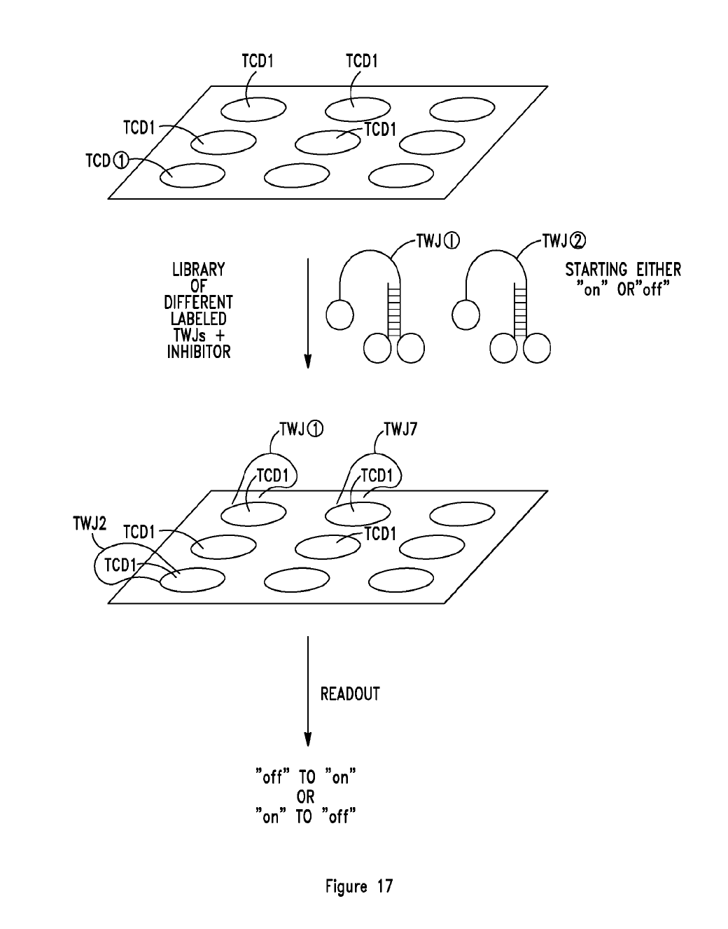

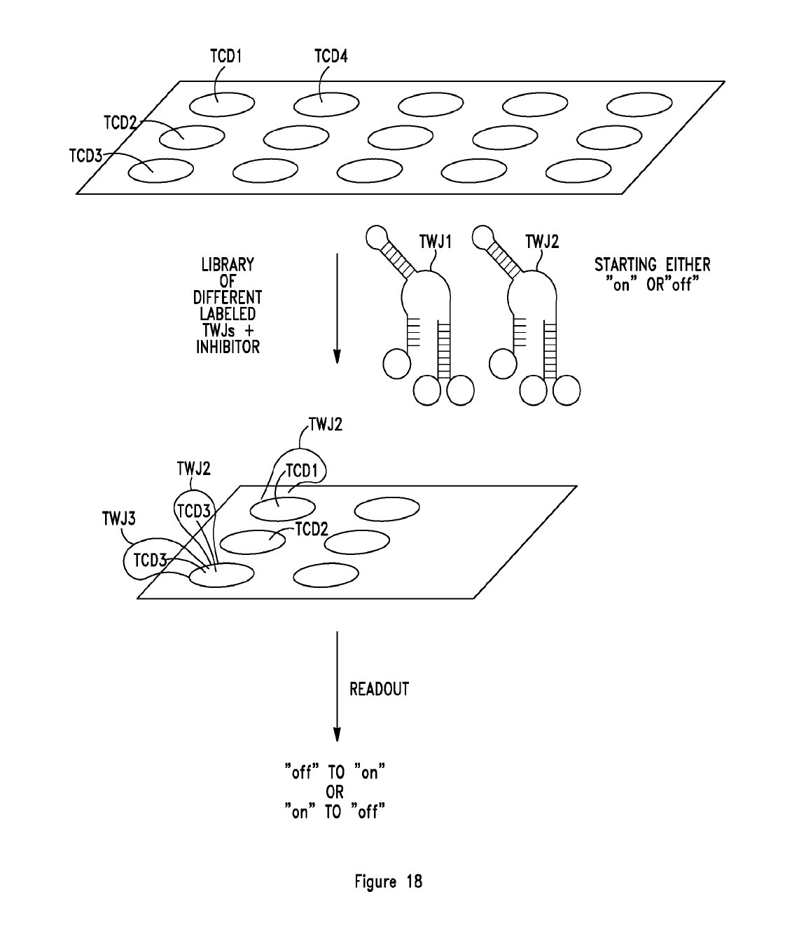

The invention includes a number of different assay formats, depending on the goal of the assay; three different formats are shown in FIGS. 16, 17 and 18 and the accompanying legends. In general, the assays fall into three formats: those that test one TCD against a number of TWJs, those that test one TWJ against a number of TCDs, and those that matrix them both, testing a library of TCDs against a library of TWJs. In some embodiments, the assays are used to screen different TCDs for activity (e.g. stability) against one or more therapeutically relevant TWJ. That is, for example, for screening for antibacterial TCDs, relevant target TWJs such as the rhoH temperature sensor of E. coli are used, and a library of TCD compounds are screened for either or both of biochemical activity (e.g. the ability to stabilize the TWJ) and/or cytotoxic activity (e.g. the ability to prevent expression of heat shock proteins and/or prevent, reduce or eliminate bacterial growth). In some cases, one or more control TWJs are used, for example, important human TWJs in a bacterial screen, to find TCD compounds that are sequence specific to the pathogen and are less likely to stabilize human TWJs.

In some embodiments, assays are run to find TCDs that preferentially bind to one of RNA and DNA over the other, that is, TCDs that bind preferentially to RNA over DNA or to DNA over RNA.

In general, the assay methods are designed to find and/or determine the sequence specificity of different TCDs and/or find TCDs that specifically bind to a particular TWJ of therapeutic relevance. The assays generally rely on adding one or more TCDs to one or more TWJs and then determining the change in FRET status. That is, as outlined in FIG. 14, an assay can be designed to start with a quenched (“off”) FRET pair in the inhibitor complex, with binding of the TCD disturbing the structure to now result in an “on” FRET status, or vice versa. It is by measuring the change in FRET status that the binding of a TCD to a TWJ is determined.

As will be appreciated by those in the art, when done in an array format, the extra substrates can be washed away, leaving only the complex of TCD:TWJ at the array site. The identity and structure of the TCD is known as it was placed and/or synthesized on the support. The identity and structure of the TWJ can be done in a number of ways, for example by using an extra label (e.g. a distinct fluorophore, preferably one who's emission and/or excitation spectra is distinguishable from the FRET pairs), or by heating the sample to disassociate the TCD from the TWJ, removing the TWJ, and sequencing it. As will be appreciated by those in the art, the sequencing can be done in any number of ways, including nucleic acid sequencing. Alternatively, the extra label can actually be a unique nucleic acid tag that is part of the TWJ substrate sequence, that can be hybridized to a secondary array for identification purposes.

In some embodiments, the screening techniques are done on solid supports, generally in array formats as discussed herein, using multimode plate readers to rapidly screen libraries of compounds. The initial concept has been tried, the results of which are shown in the Figures. This assay can be run in small volumes, for example volumes as low as 12 microliters, and is amenable to high throughput screening with fluorescence plate readers. As shown in Example 2, good results have been achieved from studies using a sigma32 mRNA with this assay and full-length sigma32 will be done.

In some embodiments, the present invention finds use in screening methods for TCDs that induce cytotoxicity in cells, including mammalian and bacterial cells. Mammalian cells, whether primary cells or cell lines, may be screened with different TCDs for cytotoxic TCDs. In general, any mammalian cells can be used with rodent, monkey and human cells being of particular use in some embodiments. As shown in the examples, TCDs can be cytotoxic even against resistant cancerous cell lines.

In some embodiments, for example similar to the heat shock response experiments outlined in Example 2, bacterial cells can be screened for TCDs that can preferentially bind bacterial TWJs (including RNA TWJs), as these are used in many bacterial strains as regulatory structures. These TCDs can then also be run against mammalian cells (particular of a human host) to determine preferential binding activity.

In some embodiments, the methods and compositions of the invention can be used to screen for TCDs that preferentially bind viral TWJs. In this embodiment, the TCDs are contacted with host cells (generally mammalian) that harbor a viral strain, and the effect of the TCD on the viral viability is measured.

### VI. Synthetic Methods

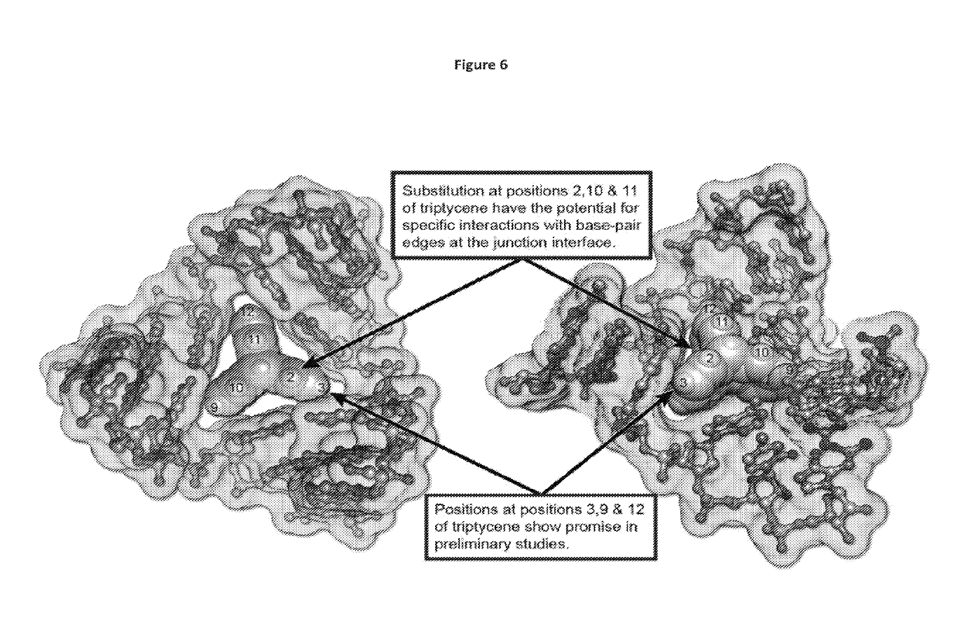

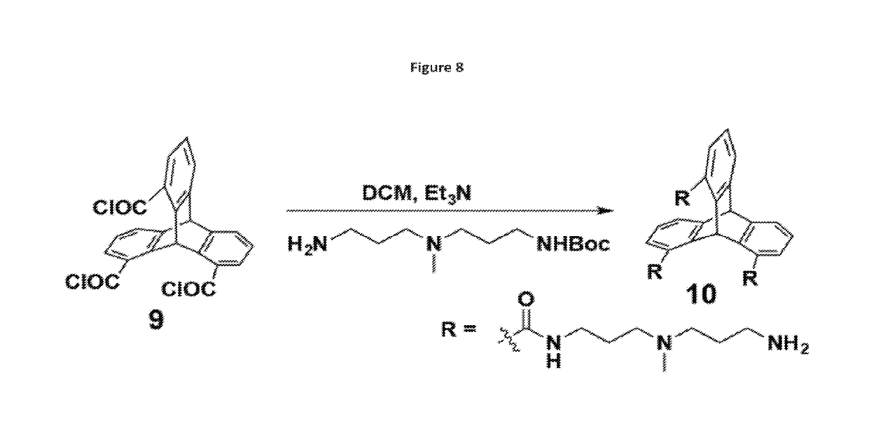

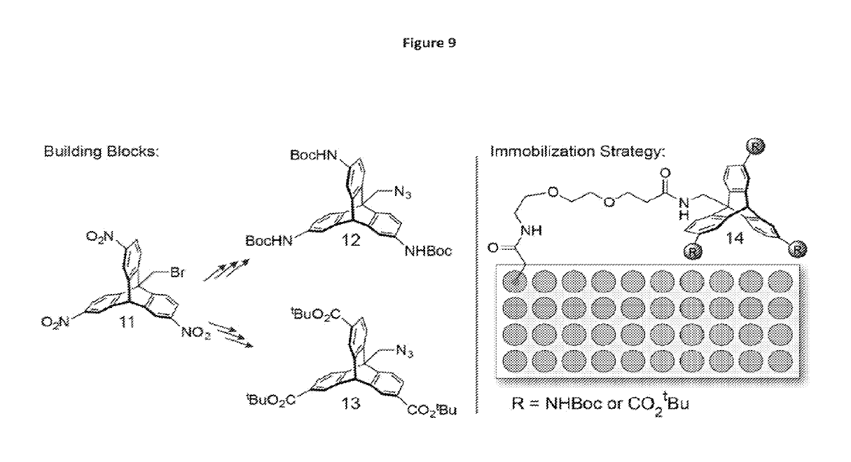

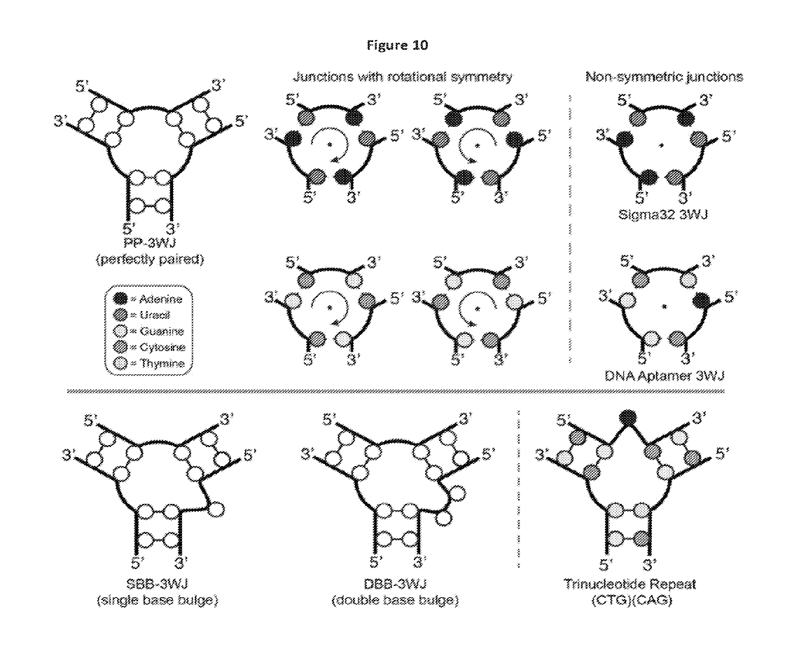

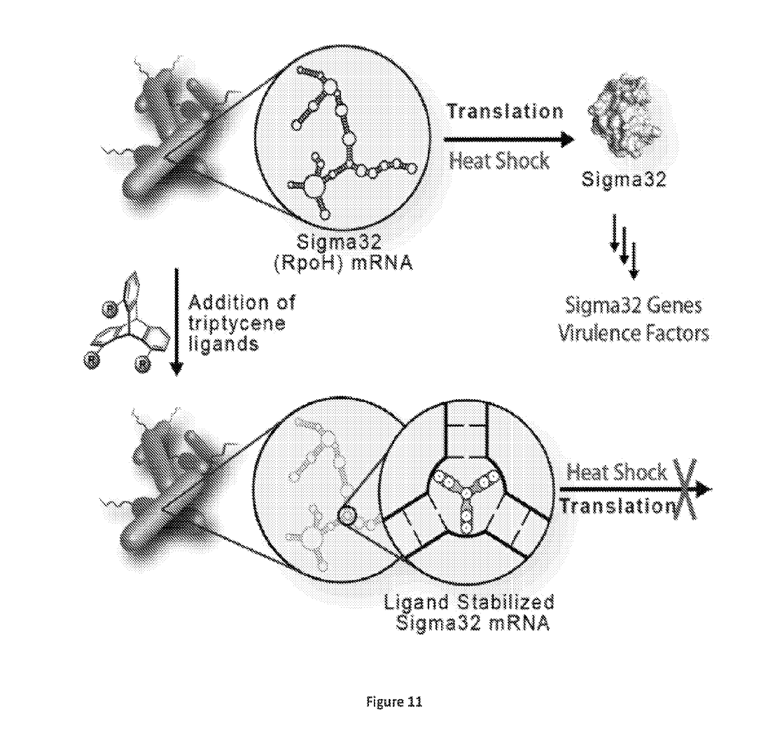

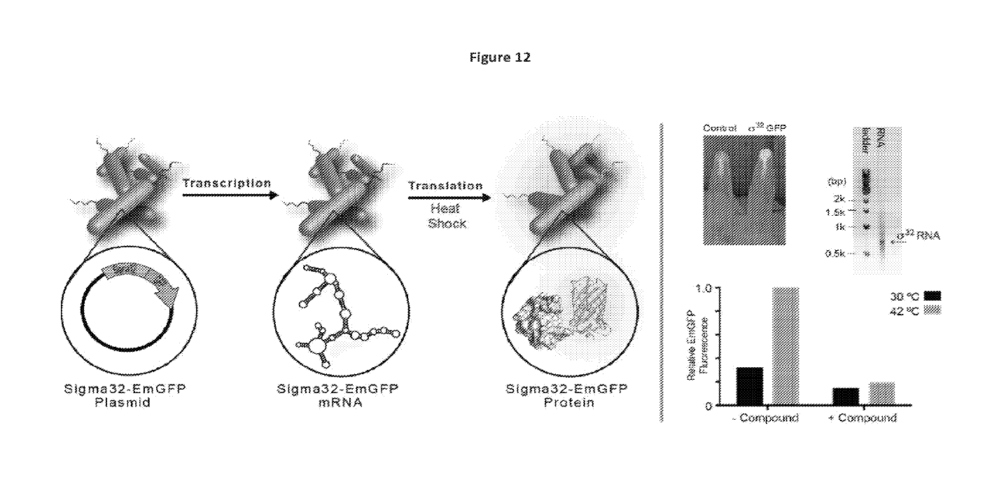

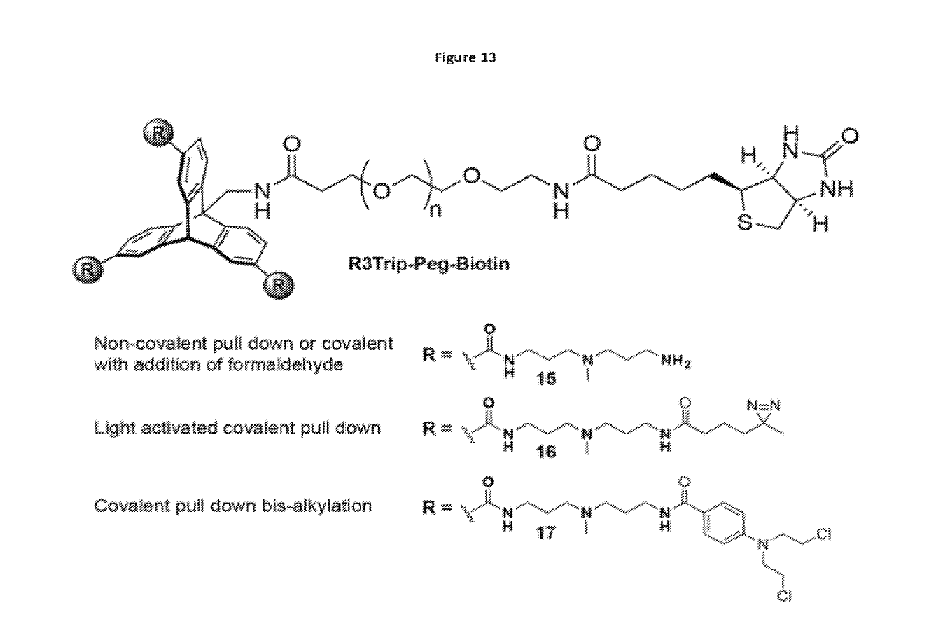

New triptycene core molecules (TCDs) are made with functionality in either the 3, 9, 12 or 2, 10, 11 positions as shown in FIGS. 3, 6 and 8. FIG. 7 shows an example of a small focused library based on commercially available natural and unnatural amino acids. The members of the library were chosen based on the most commonly occurring amino acids found at nucleic acid protein interfaces in addition to common functional groups found in small molecule RNA binders. Additionally, we chose a range of basic heterocycles and nitrogenous bases with pKa values ranging from ˜1 to 13. We also chose this library based on functionality that could participate in hydrogen bonding interactions with nucleobase edges. The opposite stereochemistry for each chiral side chain will also be synthesized to assess the importance of stereochemistry. The library in FIG. 7 allows us to ask questions about basic amine functionality in addition to the possibility of a secondary derivatization step of the final molecule to acylate the amines for further diversification. Since the nucleic acid junctions are chiral receptors, it is important to look at both D and L amino acids as enantiomeric compounds may have completely different junction specificity profiles. These core molecules are utilized to make libraries of triptycene molecules by standard coupling chemistry with functionalized amines and amino acids. This library allows us to gain insight into substituent recognition and specificity. Biophysical methods are used to characterize the interactions with nucleic acid junctions in addition to methods already developed and methods for high throughput assays. Molecules that are specific for certain junction motifs and sequences are identified and junction binding ability is cross referenced for each different junction so as to build a database of junction preference and specificity for each compound.

New trisubstituted triptycenes are synthesized to probe the influence of triptycene substitution pattern on structure and sequence specificity. Starting with commercially available 1,8 dichloroanthraquinone as reported in the literature, 1,8-bis(methoxycarbonyl)-anthracene has been previously accessed in 4 steps and 38% yield. 2-amino-6-methoxycarbonyl benzoic acid can be synthesized from commercially available 3-nitrophthalic acid via a Fischer esterification, followed by a palladium on carbon hydrogenation to reduce the nitro group. The central step in this scheme is a Diels-Alder reaction between the anthracene derivative and a benzyne equivalent, which is generated in situ by diazotization of anthranilic acid derivatives. In some embodiments, a modified, more efficient synthesis by utilizing a combined Heck coupling/benzyne Diels-Alder strategy is used. The new triptycene building block is further diversified on solid phase with short di- and tripeptides.

In some embodiments, the invention provides one or more sets of versatile orthogonally protected triptycene building blocks as shown in the Examples 4 and 5. These building blocks based on bridgehead-substituted triptycenes are used to immobilize our triptycene core structures on solid substrate spot arrays to create large immobilized libraries for diversification and screening against biologically relevant RNA targets that are either fluorophore or radio labeled.

While the invention will be described in conjunction with the following exemplary embodiments/Examples in order to explain certain principles of the invention and their practical application, thereby enabling those skilled in the art to make and utilize various exemplary embodiments of the present invention. It will be understood that the present description is not intended to limit the invention(s) to those Examples. On the contrary, the invention(s) is/are intended to cover not only the exemplary embodiments, but also various alternatives, modifications, equivalents and other embodiments, which are included within the spirit and scope of the invention as defined by the appended claims.

### EXAMPLES

### Example 1

### Recognition of Nucleic Acid Junctions Using Triptycene Based Molecules

Figures and references are as published in Barros et al., Angew Chem Int Ed Engl., 2014(53):13746, published on Sep. 24, 2014.

Nucleic acid modulation by small molecules is an essential process across the kingdoms of life. Targeting nucleic acids with small molecules represents a significant challenge at the forefront of chemical biology. Nucleic acid junctions are ubiquitous structural motifs in nature and in designed materials. Herein, we describe a new class of structure specific nucleic acid junction stabilizers based on a triptycene scaffold. Triptycenes provide significant stabilization of DNA and RNA three-way junctions, providing a new scaffold for building nucleic acid junction binders with enhanced recognition properties. Additionally, cytotoxicity and cell uptake data in two human ovarian carcinoma cell lines are reported.

Nucleic acid junctions are ubiquitous structural motifs, occurring in both DNA and RNA. Three-way junctions have been extensively studied by many biophysical techniques and represent important and sometimes transient structures in biological processes, such as replication and recombination while also occurring in triplet repeat expansions, which are associated with a number of neurodegenerative diseases. Nucleic acid junctions are ubiquitous in viral genomes and represent important structural motifs in riboswitches where small molecule modulation holds great potential. Three-way junctions are key building blocks present in many nanostructures, soft materials, multichromophore assemblies, and aptamer-based sensors. In the case of aptamer based sensors, DNA three-way junctions serve as an important structural motif. The ability to modulate aptamers using specific small molecules represents an important challenge for designing nucleic acid sensors, switches and devices.

Pioneering discoveries by Hannon, Coll and co-workers have demonstrated that metal helicates can bind nucleic acid junctions in addition to quadruplexes and helical motifs. Inspiring structural studies have yielded high-resolution details of nucleic acid junctions complexed with proteins and metal helicates, providing a starting point for rational structure-based design efforts. Given the ubiquity of nucleic acid junctions and the potential for many diverse applications, a better understanding of junction molecular recognition is needed in addition to an expanded toolbox of small molecule probes. Despite the vast number of important nucleic acid targets, we still lack the ability to selectively modulate predetermined nucleic acid structures using small molecules with high specificity and affinity beyond a few well-established recognition modes such as groove binding. Small molecule targeting of non-canonical nucleic acid motifs and higher-order structures represent an important challenge at the forefront of chemistry and chemical biology.

Herein, we report a new structure selective triptycene-based scaffold for targeting nucleic acid junctions. UV-Vis, circular dichroism (CD), Gel shift, and fluorescence quenching experiments were used to assess junction recognition properties. We find that triptycene-based junction binders exhibit a significant stabilization of perfectly paired DNA and RNA three-way junctions. Further, we report initial cytotoxicity and cell uptake data compared to cisplatin, in two human ovarian carcinoma cell lines.

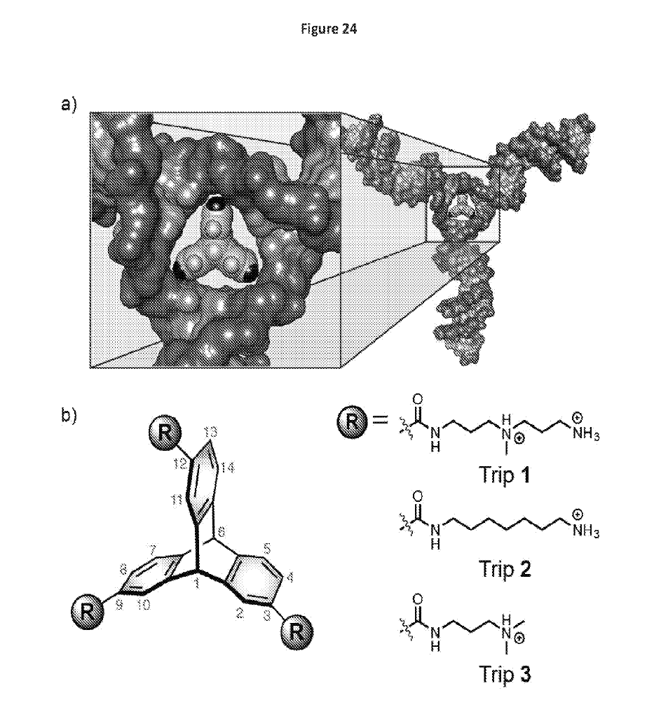

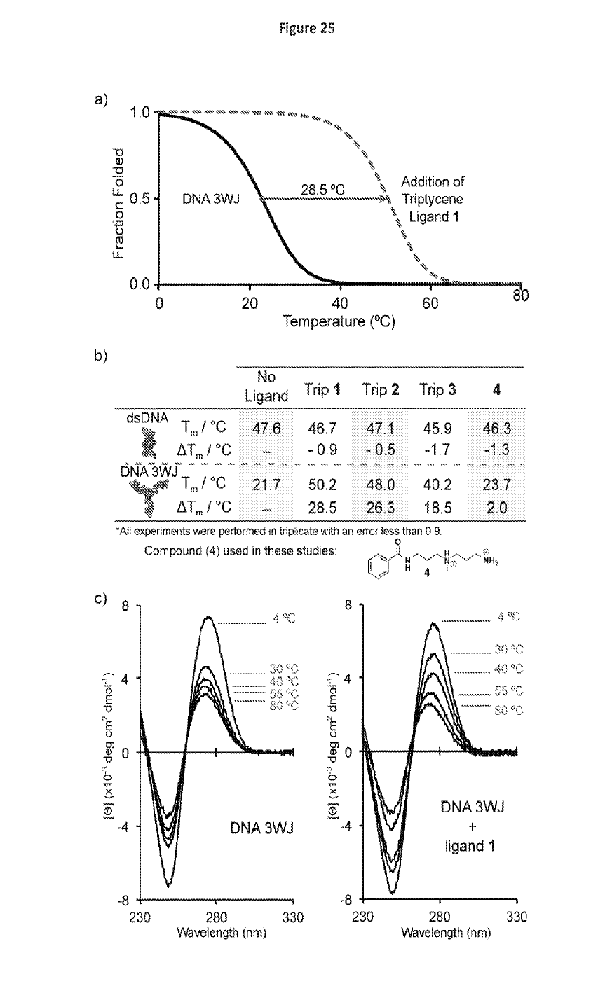

Our molecular design began with the recognition that triptycene possessed a 3-fold symmetric architecture with dimensions similar to those of the central helical interface of a perfectly base-paired nucleic acid three-way junction. Despite the occurrence of triptycene in materials applications, there is a paucity of examples where it has successfully been used for biomolecular recognition. From our analysis of the three-way junction binding site dimensions, we envisioned that the trigonal symmetry and non-planar n-surface of the aromatic rings in triptycene could potentially form stacking or buckled base pair interactions with nucleobases at the junction interface, allowing for a shape selective fit. Important to our design criteria was the choice of a non-intercalative scaffold to minimize non-specific nucleic acid binding and the triptycene structure satisfied this requirement. In previous studies, we have demonstrated that the introduction of geometric and macrocyclic constraints in nucleic acid binding small molecules can be used as a strategy to lock out common binding modes such as intercalation and groove binding. Recently, we reported that dimeric azaxanthone (diazaxanthylidene) structures lack the ability to bind B-form DNA due to their preference for an anti-folded conformation, despite the DNA binding ability of well-known monomeric xanthone intercalators. In our previous studies we attributed the lack of B-form DNA binding to the discontinuous pi-system, caused by the overcrowded anti-folded molecular architecture. This hypothesis is consistent with the architectural requirements for classical intercalator scaffolds, requiring more than one ring of continuous planar n-surface area. By a similar structural analysis, we expected the discontinuous n-surface area of triptycene to rule out classical intercalative binding modes. In addition to the shape complementarity and three-dimensional architecture of the triptycene scaffold, we were attracted to its modularity and potential for diversity. The triptycene scaffold provides up to 14 positions for diversification, three rings with four positions each in addition to two bridgehead positions. Triptycene can also be thought of as having three buckled n-faces and two three-fold symmetric edge faces with a bridgehead located at the center of each. These structural attributes of triptycene will become important for future studies as we consider topological differentiation of nucleic acid junction faces for achieving sequence specificity and distinguishing DNA from RNA junctions. Size comparison of the triptycene core relative to previously reported DNA and RNA three-way junction crystal structures confirmed our initial hypothesis regarding triptycene shape complementarity, prompting us to initiate synthetic efforts toward the first rationally designed nucleic acid junction binders based on triptycene (FIG. 24a). We synthesized triptycenes 1-3 (Trip 1-3) and evaluated their ability to discriminate a DNA 3WJ from dsDNA, using well-established UV and CD spectroscopic techniques (FIG. 25).

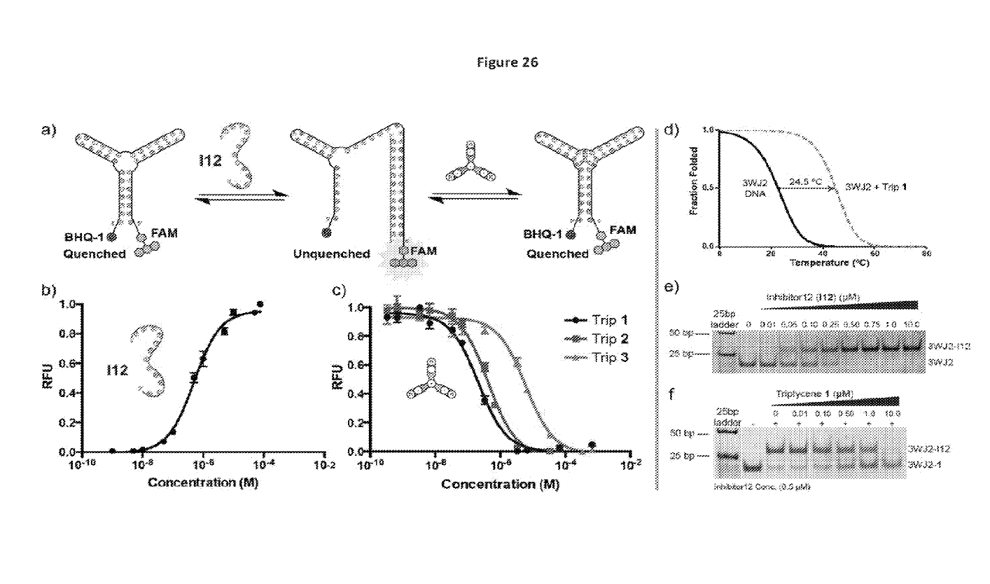

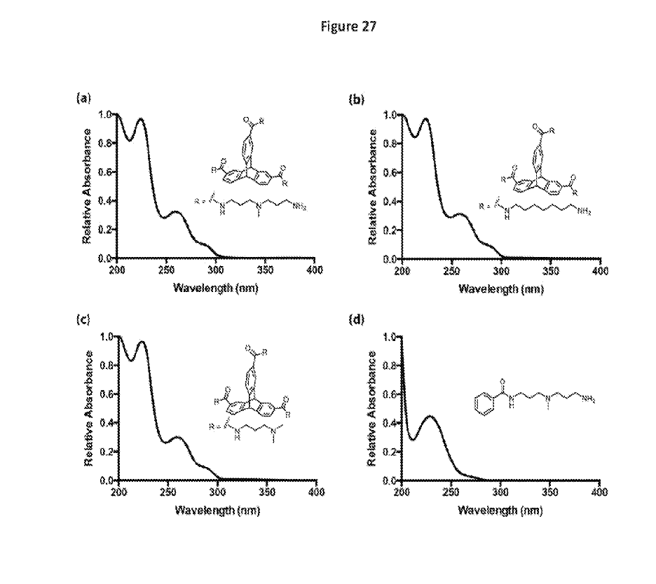

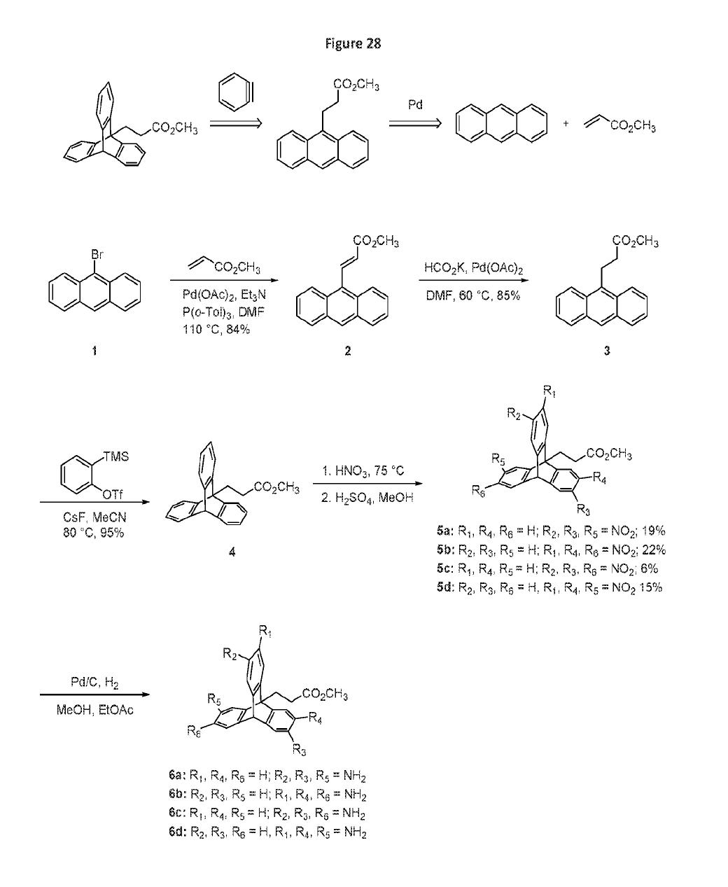

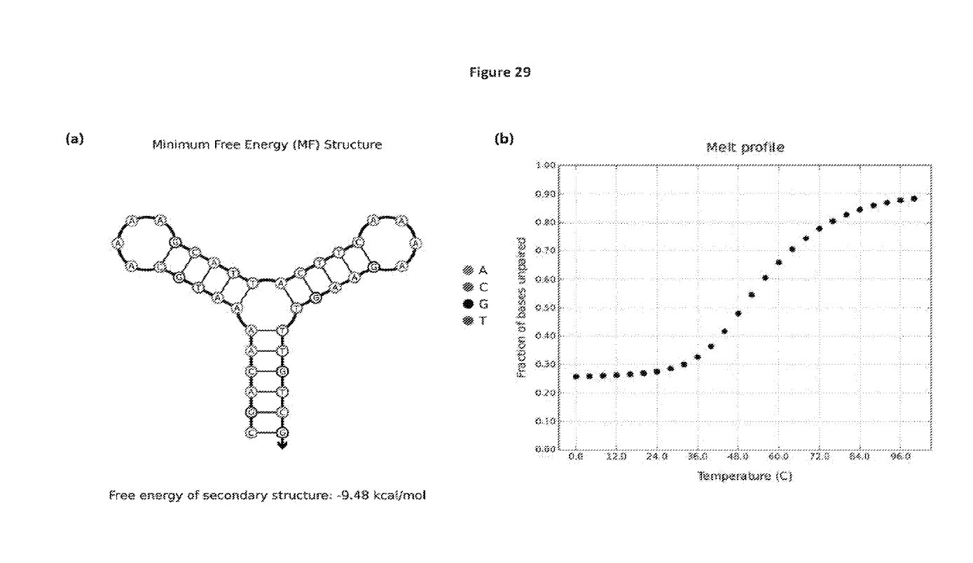

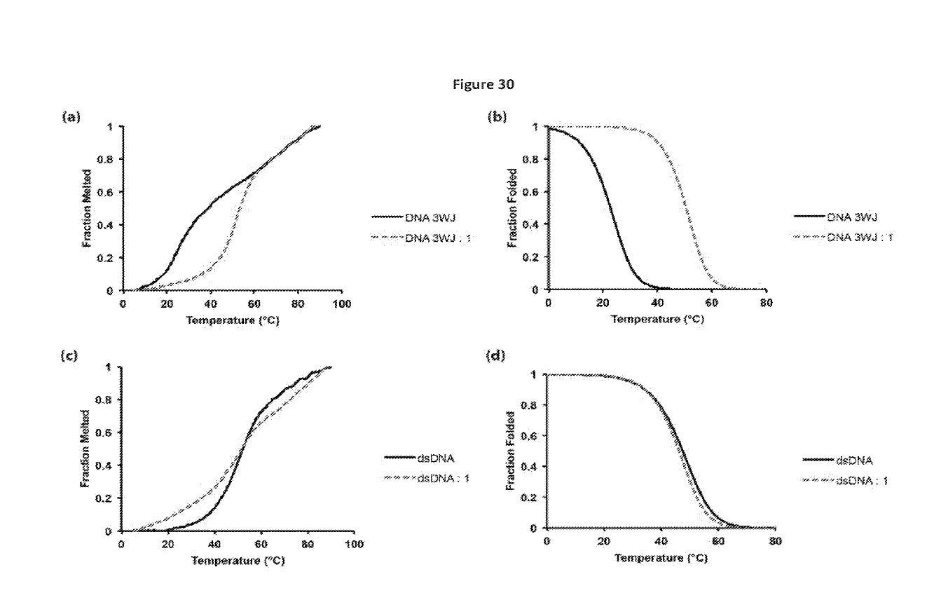

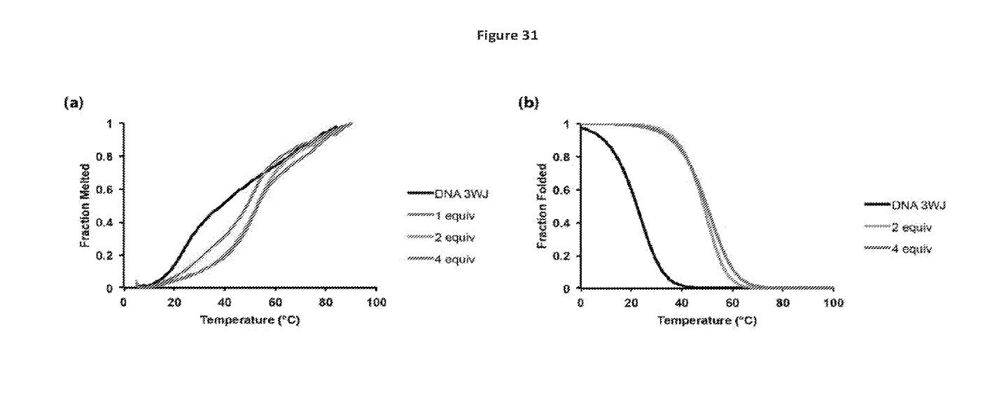

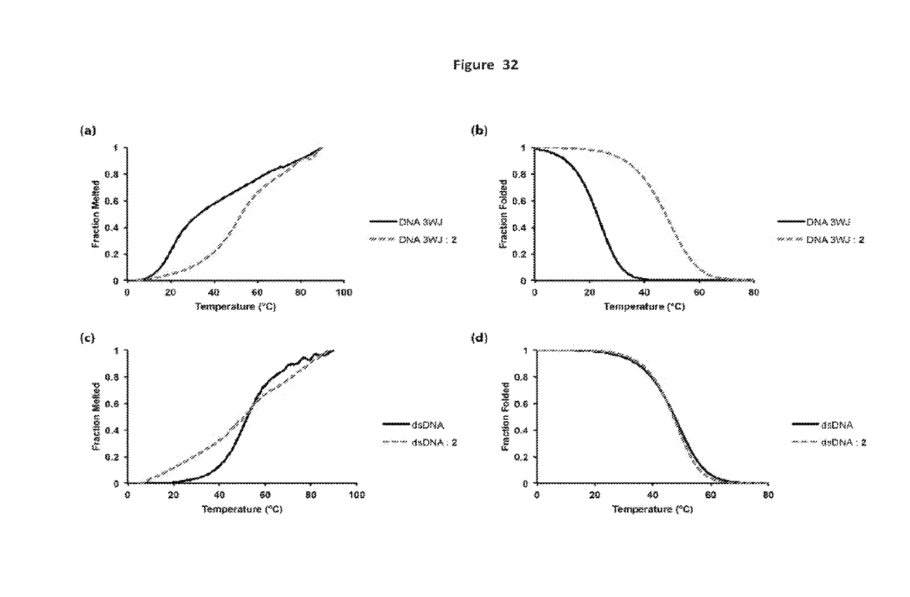

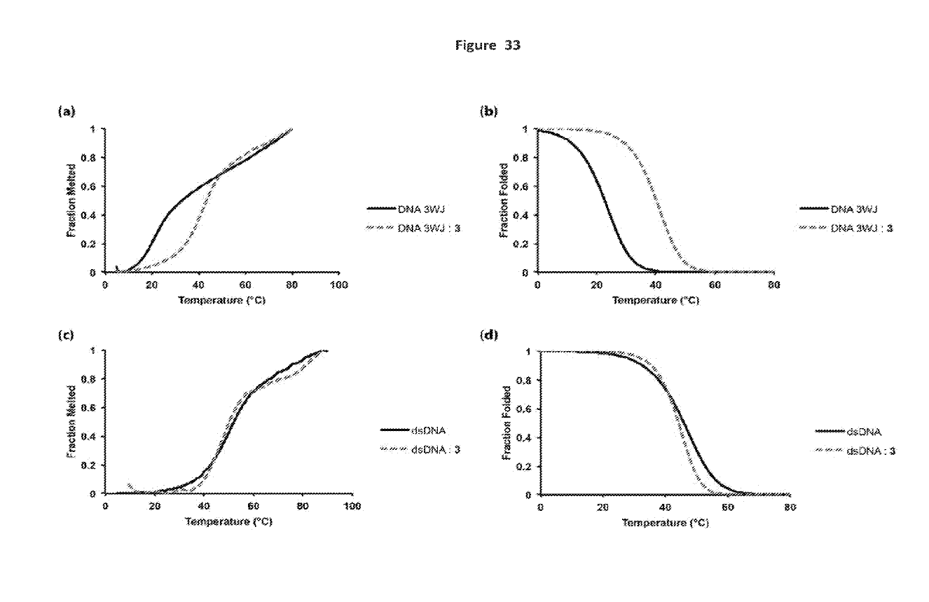

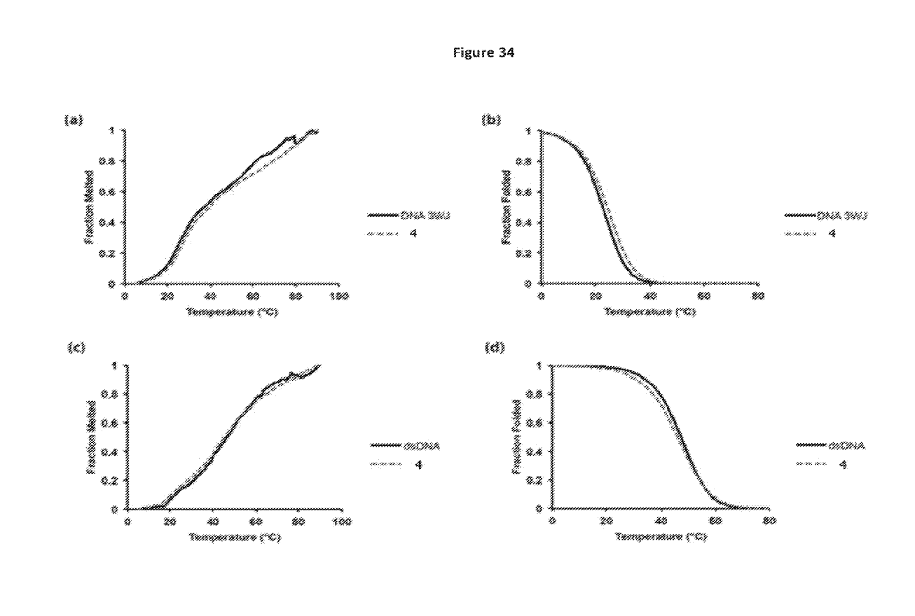

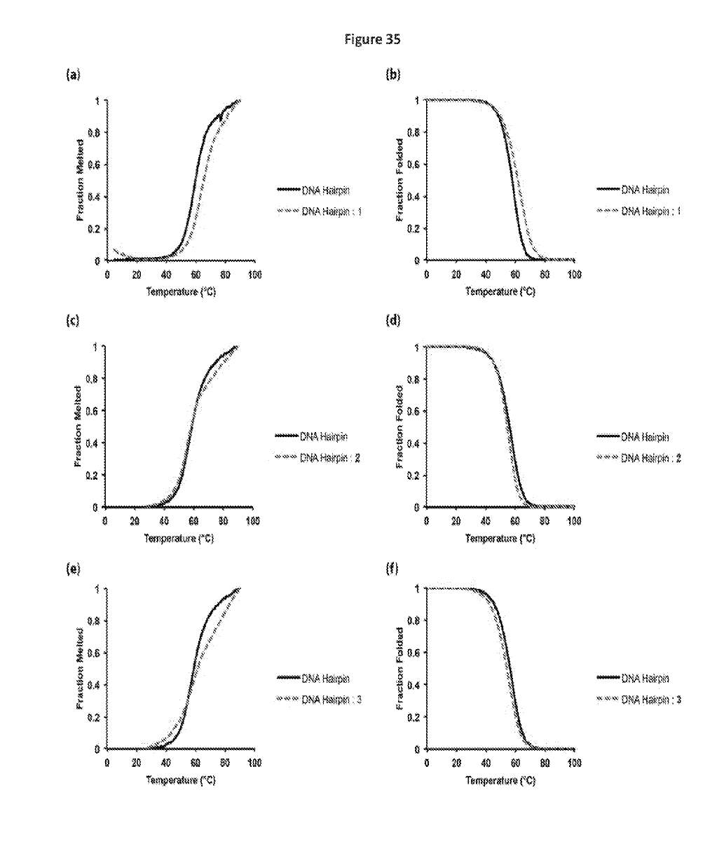

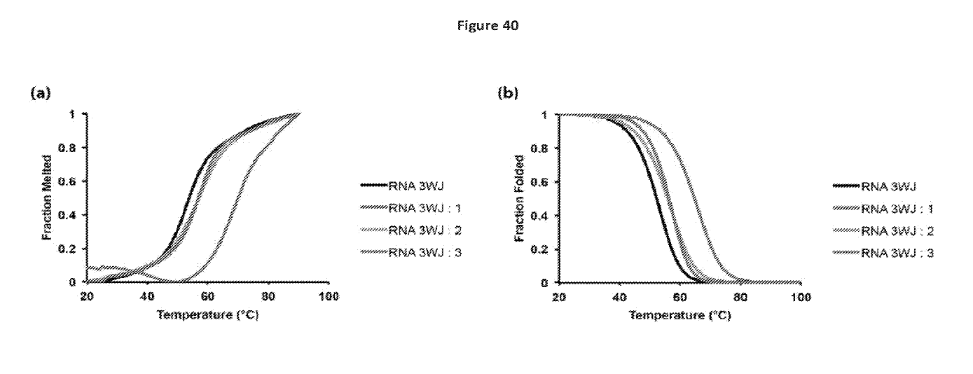

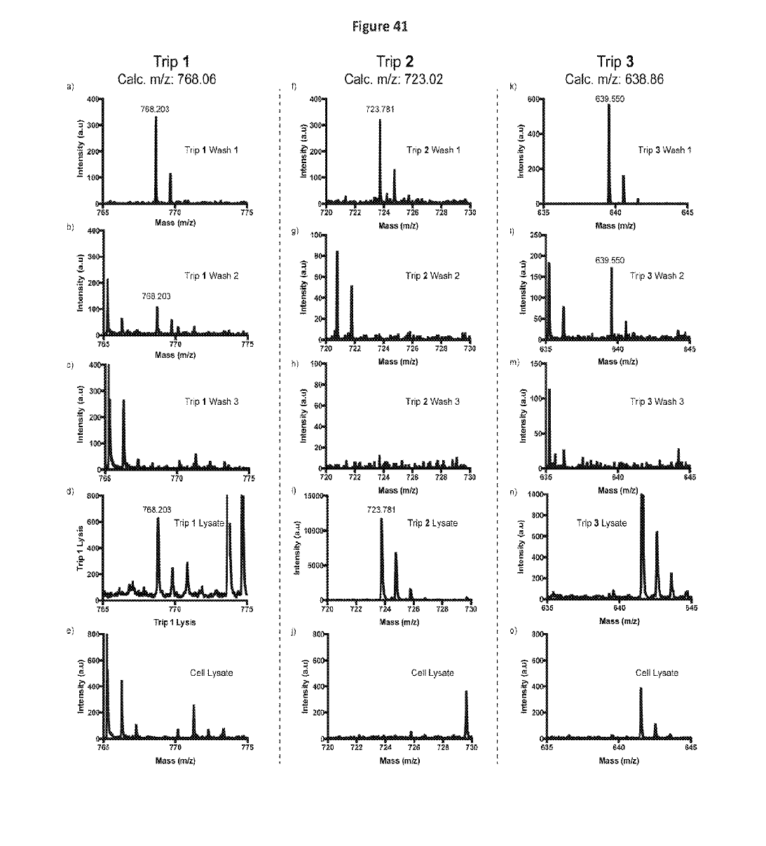

UV thermal melting experiments were performed to determine the degree of stabilization of Trip 1-3 toward a DNA 3WJ versus dsDNA (FIG. 25a,b). Triptycene 1-3 did not stabilize dsDNA even though a significant stabilization of the DNA 3WJ was observed, with ΔTₘ values of 28.5, 26.3, and 18.5° C. for Trip 1-3, respectively (FIG. 25b). Maximum stabilization was achieved after addition of slightly more than one equivalent of ligand (FIG. 31). Triptycene derivatives 1-3 also provide a comparison of the effect of positive charge and linker length on junction stabilization. Triptycene 2 and 3, where the internal and terminal amines have been removed, demonstrate that the linker length is an important structural parameter to explore in future studies. Despite the decreased number of charges in Trip 2 and 3, both compounds retain significant 3WJ stabilizing ability (FIG. 25b). As a further control experiment, stabilization of a DNA hairpin corresponding to one of the arms of the junction was evaluated and showed no significant stabilization (FIG. 35). To further study the impact of the triptycene core, compound 4 was synthesized. Compound 4 consists of the minimal repeating structural motif embedded in the trimeric architecture of triptycenes 1-3. UV melting analysis with 4 results in a ΔTₘ=2.0° C. for DNA 3WJ and a ΔTₘ=−1.3° C. for dsDNA (FIG. 25b). This lack of stabilization further demonstrates the unique structure selectivity imparted by triptycenes 1-3. Trip 1 was also evaluated against a well studied three-way junction (3WJ2) shown in FIG. 26. The stability of this junction was found to increase by 24.5° C. in the presence of Trip 1. In addition to DNA three-way junction stabilization studies we have conducted initial studies with RNA three-way junctions. Triptycenes 1-3 stabilize RNA three-way junctions by UV melting analysis. The ΔTₘ values for stabilization of the RNA 3WJ are as follows: Trip 1=12.5° C., Trip 2=2.9° C., and Trip 3=3.4° C. (FIG. 40). RNA selective junction binders can also be developed.

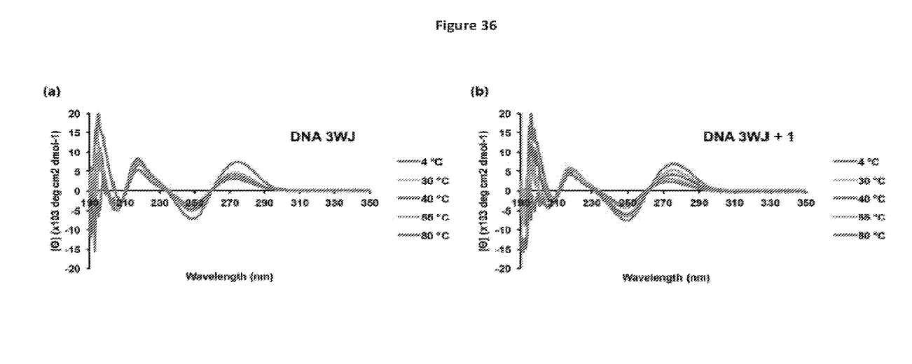

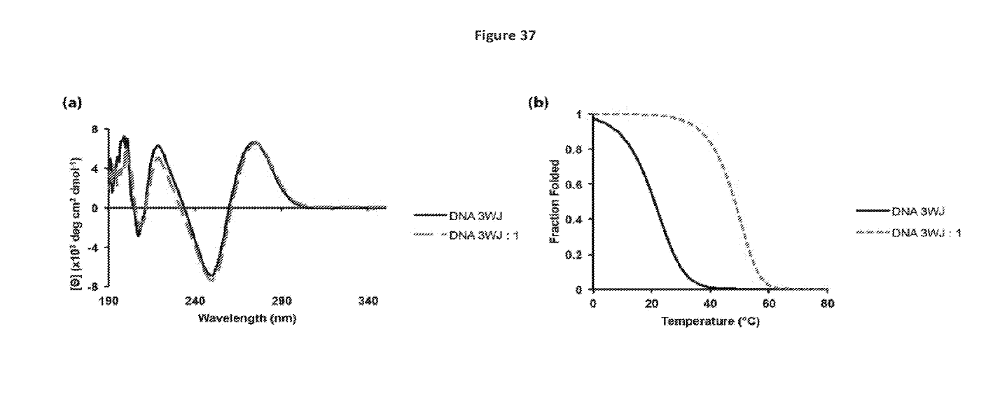

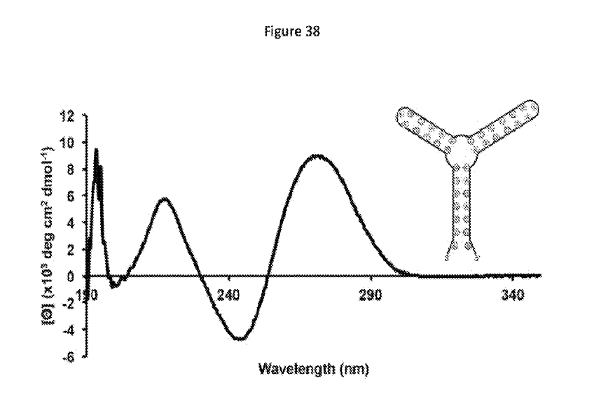

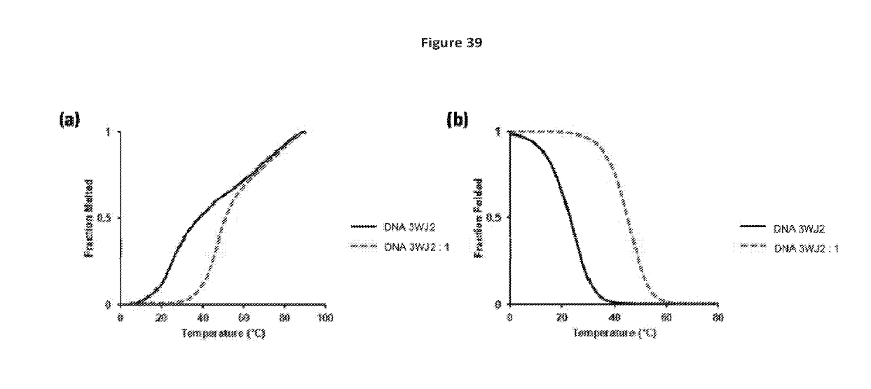

Circular dichroism (CD) was used to further explore the interaction of Trip 1 with DNA 3WJ (FIG. 25c and FIG. 36) The temperature-dependent CD spectra of the DNA 3WJ with and without Trip 1 exhibit a maximum at 275 nm and a minimum at 245 nm centered around 260 nm, which is indicative of the B-DNA helical conformation. This CD signature resembles that of other intramolecular nucleic acid junctions. As the temperature was increased from 4° C. to 80° C., the maximum at 275 nm decreased, and the minimum at 245 nm became less negative. The temperature-induced change in the CD spectrum indicates melting of the DNA 3WJ helical arms. The largest change in the spectrum for the DNA 3WJ was observed between 4° C. and 30° C. In the presence of Trip 1, the CD maximum at 275 nm decreased more gradually with increasing temperature, which is consistent with the ligand-induced stabilization observed during UV experiments. A CD thermal denaturation experiment, in which the molar ellipticity is measured at 255 nm as a function of temperature, further demonstrated the dramatic stabilization of DNA 3WJ in the presence of Trip 1. The CD ΔTₘ was determined to be 27.4° C., which is in agreement with the UV thermal denaturation results (FIG. 37).

Gel shift experiments were performed on DNA 3WJ2 to further support junction binding by 1 (FIG. 26). Addition of increasing concentrations of Trip 1 directly to DNA 3WJ2 did not result in a measurable shift using a polyacrylamide gel. This indicates that there is likely not a non-specific intercalative binding mode. To further confirm that Trip 1 binds the three-way junction, 3WJ2 was incubated with a 12 bp oligonucleotide complementary to the 5′-end of the junction (inhibitor 12). Consistent with the known DNA three-way junctions in the literature, faster migration was observed relative to the migration of duplex DNA (<25 bp), suggesting the formation of a more compact 3WJ structure. Titration of inhibitor 12 (I12) results in a slower-migrating band on the gel, indicating the formation of a larger complex (FIG. 26e). Addition of increasing concentrations of Trip 1, results in reformation of the three-way junction and displacement of the inhibitor strand (FIG. 26f).

A fluorescence quenching experiment was used to verify binding to the three-way junction. The 5′- and 3′-ends of a DNA 3WJ forming oligonucleotide were labeled with a fluorophore (FAM) and a quencher (BHQ-1), respectively (FIG. 26). Folding of the junction brings the 5′ and 3′ ends into close proximity resulting in fluorescence quenching. A 12 bp oligonucleotide, complementary to the 5′-end of the junction, was used as an inhibitor to stabilize the open state of the junction. Addition of Trip 1 to the unquenched state of the 3WJ2-I12 complex resulted in a decrease in fluorescence, indicating that the ends were brought into close proximity as a result of 3WJ formation. The apparent Kd of the triptycenes were determined to be 0.221 μM for Trip 1, 0.396 μM for Trip 2, and 5.499 μM for Trip 3 in the fluorescence displacement assay (FIG. 26a, b).

Initial studies of the cytotoxicity and cell uptake for Trip 1-3 were conducted using human ovarian carcinoma cell lines. Previous studies of metallohelicates show diverse biological activity and cytotoxic effects in cisplatin resistant A2780cis cell lines. This promising result for the metallohelicates led us to explore initial cytotoxicity studies for triptycenes 1-3 in these cell lines. Their activity was investigated in human ovarian carcinoma A2780 cells and the cisplatin-resistant A2780cis cell line using an Alamar blue assay. Significant differences in sensitivity were observed for each of the triptycenes in the two cell lines compared to cisplatin (FIG. 49a). No cytotoxic activity was observed for triptycene 3. Triptycene 1 shows similar potency to cisplatin in the A2780 cell line, but demonstrated increased cytotoxicity against the A2780cis cell line compared to cisplatin. The highest potency was observed for triptycene 2, with a complete loss of cell viability observed for A2780 cells and only 6% viability for A2780cis cells. Triptycenes 1-3 show very promising anticancer activity, demonstrating increased or similar potency to cisplatin in the cell lines tested. We conducted cellular uptake studies in A2780 cells and demonstrate that triptycenes can be efficiently internalized within two hours post treatment. Trends in cellular uptake parallel our initial cytotoxicity data with the most potent compound Trip 2 showing the greatest cellular uptake, followed by Trip 1 and Trip 3 (FIG. 49b).

In summary, we have rationally designed a new class of non-intercalative triptycene-based nucleic acid junction binders. We have demonstrated that triptycene-based molecules have the ability to recognize both DNA and RNA three-way junctions, providing a new versatile scaffold for targeting higher-order nucleic acid structure. Initial biological studies show promising cytotoxicity in cisplatin resistant human ovarian carcinoma cell lines and positive cellular uptake. Ongoing efforts in our laboratory are directed toward the recognition of both DNA and RNA junctions in addition to the development of new classes of structure specific nucleic acid modulators. Junctions are one of the most ubiquitous structural motifs and many exciting opportunities exist for developing small molecule modulators of higher-order structures such three-way and four-way junctions. Seminal studies on four-way junctions have paved the way for recent important contributions where selective small molecule modulators are being developed. Small molecule targeting of non-canonical nucleic acid motifs and higher-order structures represent an important challenge at the forefront of chemistry and chemical biology with many exciting opportunities.

General Methods:

DNA 3WJ (5′-CGA CAA AAT GCA AAA GCA TTA CTT CAA AAG AAG TTT GTC G-3′), duplex DNA (5′-CCAGTACTGG-3′), DNA 3WJ2 (5′-GGG AGA CAA GGA AAA TCC TTC AAT GAA GTG GGT CGA CA-3′), and DNA hairpin (5′-CAA AAT GCA AAA GCA TTT TG-3′) were purchased from Integrated DNA Technologies (IDT). HPLC-purified DNA 3WJ2 oligo modified with a 5′-FAM and a 3′-BHQ-1 was purchased from IDT. The DNA 3WJ was predicted to have a cooperative single inflection melting curve using NUPACK. Trans-Dichlorobis(triphenylphospine)palladium(II) was purchased from Strem Chemicals, Inc. (Newburyport, Mass., USA). Sodium hydroxide was purchased from Fisher Scientific (Pittsburgh, Pa., USA). (1-[Bis(dimethylamino)methylene]-1H-1,2,3-triazolo[4,5-b]pyridinium 3-oxid hexafluorophosphate) (HATU) was purchased from GenScript (Piscataway, N.J., USA). Benzoic acid was purchased from Acros Organics. All other reagents were purchased from Sigma Aldrich (St. Louis, Mo., USA) and used without further purification. Reactions requiring anhydrous conditions were run under argon with solvents purchased from Fisher dried via an alumina column. Silicycle silica gel (55-65 A pore diameter) was used for silica chromatography. Thin-layer chromatography was done using Sorbent Technologies (Norcross, Ga., USA) silica plates (250 μm thickness). Milli-Q (18 MΩ) water was used for all solutions (Millipore; Billerica, Mass., USA).

¹H and ¹³C NMR were recorded on a Bruker UNI 500 NMR at 500 and 125 MHz, respectively. Matrix-assisted laser desorption ionization (MALDI) mass spectra were obtained on a Bruker Ultraflex III MALDI-TOF-TOF mass spectrometer (Billerica, Mass., USA) using a-cyano-4-hydroxycinnamic acid (CHCA). Low resolution electrospray ionization (ESI) mass spectra (LRMS) were collected on a Waters Acquity Ultra Performance LC (Milford, Mass., USA). High resolution mass spectra were obtained at the Univeristy of Pennsylvania Mass Spectrometry Center on a Micromass AutoSpec electrospray/chemical ionization spectrometer. Ultraviolet absorption spectroscopy and thermal denaturation experiments were performed on a JASCO V-650 spectrophotometer (JASCO Analytical Instruments; Easton, Md., USA) equipped with a JASCO PC-734R multichannel Peltier using quartz cells with 1 cm path lengths. High-performance liquid chromatography was performed on a JASCO HPLC (Easton, Md., USA) equipped with a Phenomenx (Torrance, Calif., USA) column (Luna 5μ C18(2) 100 A; 250×4.60 mm, 5 μm) using aqueous (H2O+0.1% CF3CO2H) and organic (CH3CN) phases. Circular dichroism experiments were performed on an Aviv 410 CD spectrometer (Aviv Biomedical; Lakewood, N.J., USA) using a 0.1 cm path length quartz cuvette. Fluorescence measurements were collected on a Tecan M1000 plate reader (Mannedorf, Switzerland).

HPLC chromatograms were obtained at all wavelengths from 200 to 800 nm (bottom plot). 254 and 214 nm were chosen as virtual channels to show the absorbances at those two specific wavelengths (top plot). The blue line corresponds to 254 nm and the red line corresponds to 214 nm (top plot). The lamps used were D2+W with a slit width of 4 nm. A flow rate of 1.00 mL/min was used over 30 minutes. The method began at 10% acetonitrile and 90% water+0.1% TFA. The gradient was slowly increased to 100% acetonitrile.

Synthesis:

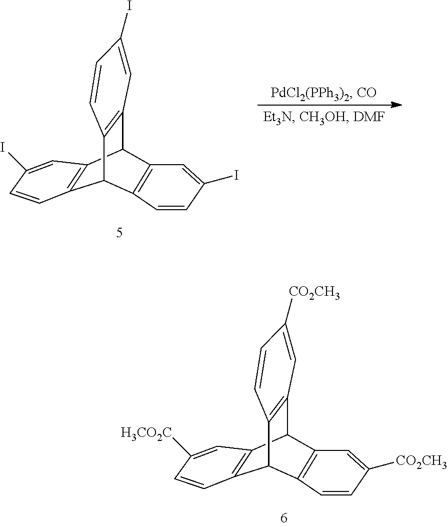 [SMILES: Ic1ccc2c(c1)C1c3cc(I)ccc3C2c2ccc(I)cc21 ; COC(=O)c1ccc2c(c1)C1c3cc(C(=O)OC)ccc3C2c2ccc(C(=O)OC)cc21]

### Trimethyl 9,10-dihydro-9,10-[1,2]benzenoanthracene-2,7,15-tricarboxylate (6)

2,7,15-triiodo-9,10-dihydro-9,10-[1,2]benzenoanthracene (5) was synthesized according to previously described method, C. Zhang, C. F. Chen, J. Org. Chem. 2006, 71, 6626-6629, hereby expressly incorporated herein by reference for the methods, figures and legends herein. To a solution of 5 (23.6 mg, 0.037 mmol) in anhydrous DMF (0.5 mL) was added PdCl₂(PPh₃)₂ (1 mg, 0.0014 mmol), Et₃N (27.2 mg, 0.27 mmol) and CH₃OH (0.2 mL). The solution was stirred under CO (150 psi) in a Parr apparatus at 60° C. for 14 hours. The reaction mixture was concentrated under vacuum and purified by column chromatography on silica gel with 30% dichlromethane/hexanes. The product was isolated as a yellow solid (15.9 mg, 0.037 mmol, 60%). ¹H NMR (500 MHz, CDCl₃) δ 8.07 (s, 3H), 7.77-7.75 (m, 3H), 7.48-7.46 (d, 3H), 5.62 (s, 1H), 5.59 (s, 1H), 3.87 (s, 9H); ¹³C NMR (500 MHz, CDCl₃) δ 166.77, 148.52, 144.80, 128.01, 127.96, 124.98, 124.12, 54.29, 53.74, 52.26; HRMS m/z calcd for C₂₆H₂₀O₆ [M−H] 427.1259, observed [M−H] 427.1201.

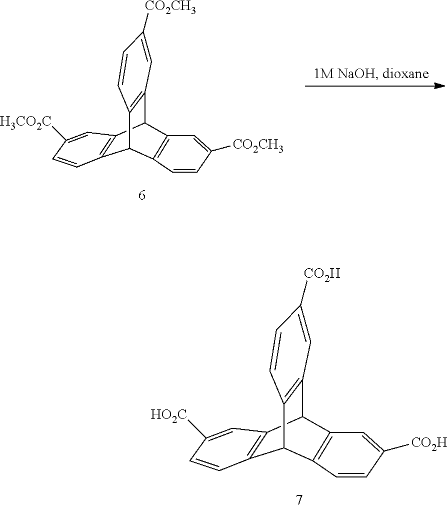 [SMILES: COC(=O)c1ccc2c(c1)C1c3cc(C(=O)OC)ccc3C2c2ccc(C(=O)OC)cc21 ; O=C(O)c1ccc2c(c1)C1c3cc(C(=O)O)ccc3C2c2ccc(C(=O)O)cc21]

### 9,10-dihydro-9,10-[1,2]benzenoanthracene-2,7,15-tricarboxylic acid (7)

To a solution of 6 (9.6 mg, 0.019 mmol) in dioxane (0.2 mL) was added 1 M NaOH (0.2 mL) and heated to 45° C. for 2 hours. Dioxane was removed under vacuum, diluted with water and extracted with CH₂Cl₂. The aqueous phase was acidified with 1 M HCl to pH 2 and the solution was extracted with EtOAc. The organic phase was dried over Na₂SO₄, and concentrated under vacuum to leave 7 (7.1 mg, 0.018 mmol, 95%) as a white solid. ¹H NMR (500 MHz, MeOD) δ 8.10 (d, 3H), 7.77-7.75 (m, 3H), 7.56-7.55 (d, 3H), 5.82 (s, 1H), 5.80 (s, 1H). ¹³C NMR (500 MHz, MeOD) δ 169.58, 150.37, 146.61, 129.61, 128.97, 125.85, 125.01, 55.11, 54.57; HRMS m/z calcd for C₂₃H₁₄O₆ [M−H] 385.079, observed 385.0725.

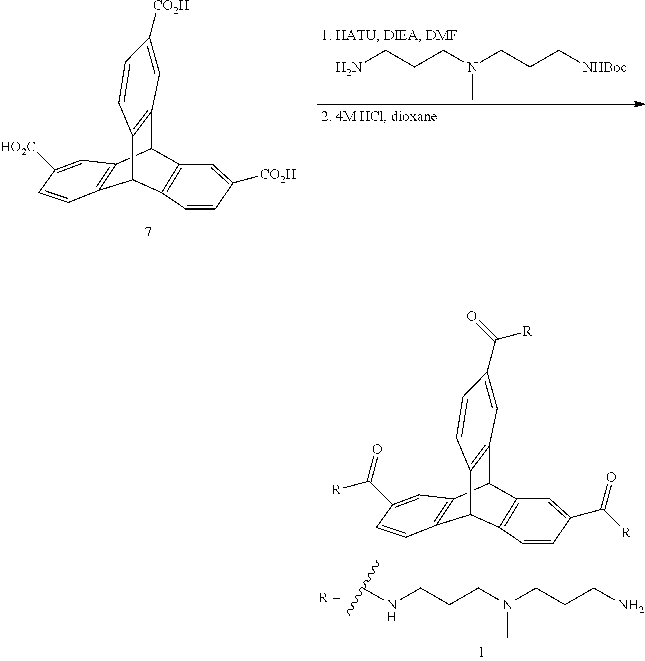

### N²,N⁶,N¹⁴-tris(3-((3-aminopropyl)(methyl)amino)propyl)-9,10-dihydro-9,10-[1,2]benzenoanthracene-2,6,14-tricarboxamide (1)

To a solution of 7 (7 mg, 0.018 mmol) in DMF (0.2 mL) was added HATU (24 mg, 0.063 mmol) and DIEA (15.5 mg, 0.12 mmol) and was stirred at room temperature for 5 min. Tert-butyl (3-((3-aminopropyl)(methyl)amino)propyl)carbamate (15.5 mg, 0.063 mmol) was added and stirred overnight. The reaction mixture was concentrated under vacuum, then diluted with water and extracted with EtOAc. The organic phase was dried over Na₂SO₃ and concentrated. Crude product was suspended in 4 M HCl in dioxane and stirred for 2 hours. The mixture was concentrated, redissolved in acidic water (0.1% TFA) and washed with CH₂Cl₂. Product was purified on a JASCO High-Performance Liquid Chromatography (HPLC) instrument on a C18 column using aqueous (H₂O+0.1% TFA) and organic (CH₃CN) phases. ¹H NMR (500 MHz, D₂O) δ 7.87 (s, 3H), 7.60 (d, 6H), 5.92 (s, 1H), 5.89 (s, 1H), 3.45-3.44 (m, 6H), 3.31-3.17 (m, 12H), 3.09-3.06 (t, 6H), 2.89 (s, 9H), 2.16-2.03 (m, 12H); ¹³C NMR (500 MHz, D₂O) δ 170.55, 147.95, 145.03, 131.01, 125.19, 124.34, 122.54, 117.46, 115.14, 112.82, 53.97, 52.86, 52.69, 39.54, 36.59, 36.38, 23.83, 21.87; HRMS m/z calcd for C₄₄H₆₅N₉O₃ [M+H] 768.521, observed 768.5291.

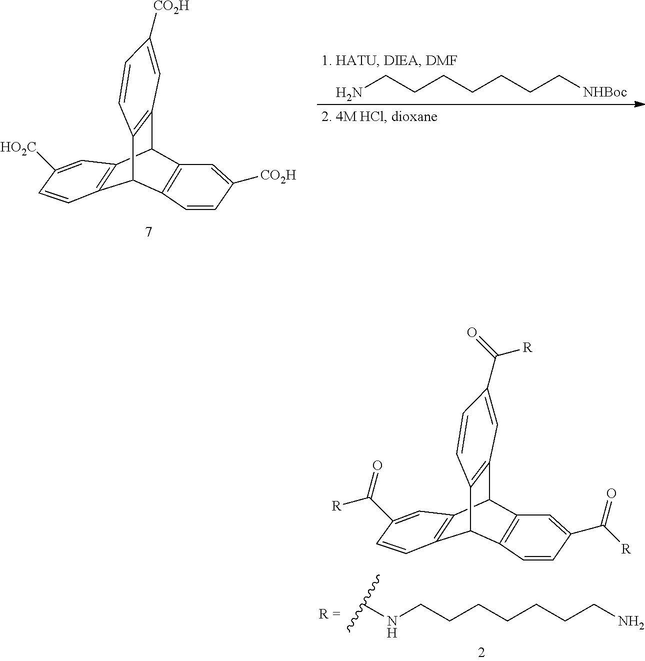

### N²,N⁶,N¹⁴-tris(7-aminoheptyl)-9,10-dihydro-9,10-[1,2]benzenoanthracene-2,6,14-tricarboxamide (2)

To a solution of 7 (4.5 mg, 0.012 mmol) in DMF (0.2 mL) was added HATU (14.2 mg, 0.037 mmol) and DIEA (9.0 mg, 0.070 mmol) and was stirred at room temperature for 5 min. tert-butyl (7-aminoheptyl)carbamate (8.5 mg, 0.037 mmol) was added and stirred overnight. The reaction mixture was concentrated under vacuum, then diluted with water and extracted with EtOAc. The organic phase was dried over Na₂SO₃ and concentrated. Crude product was suspended in 4 M HCl in dioxane and stirred for 2 hours. The mixture was concentrated, redissolved in acidic water (0.1% TFA) and washed with CH₂Cl₂. Product was purified on a JASCO High-Performance Liquid Chromatography (HPLC) instrument on a C18 column using aqueous (H₂O+0.1% TFA) and organic (CH₃CN) phases. ¹H NMR (500 MHz, D₂O) δ 7.80 (s, 3H), 7.61-7.60 (d, 3H) 7.47-7.46 (d, 3H), 5.90 (s, 1H), 5.79 (s, 1H), 3.36-3.33 (t, 6H), 2.92-2.89 (t, 6H), 1.61-1.58 (m, 12H), 1.34 (m, 18H); ¹³C NMR (500 MHz, D₂O) δ 169.20, 147.55, 144.77, 131.30, 124.15, 117.56, 115.24, 39.91, 39.362, 28.62, 27.91, 26.65, 26.00, 25.48; HRMS m/z calcd for C₄₄H₆₂N₆O₃ [M+H] 723.4848, observed 723.4964.

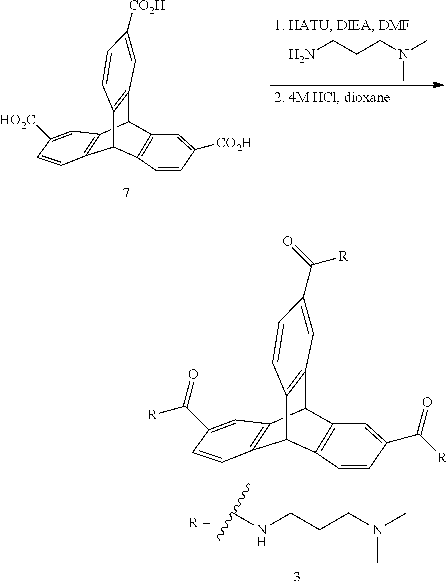

### N²,N⁶,N¹⁴-tris(3-(dimethylamino)propyl)-9,10-dihydro-9,10-[1,2]benzenoanthracene-2,6,14-tricarboxamide (3)

To a solution of 7 (4.2 mg, 0.011 mmol) in DMF (0.2 mL) was added HATU (13.2 mg, 0.035 mmol) and DIEA (8.7 mg, 0.068 mmol) and was stirred at room temperature for 5 min. N¹,N¹-dimethylpropane-1,3-diamine (8.7 mg, 0.035 mmol) was added and stirred overnight. The reaction mixture was concentrated under vacuum, then diluted with water and extracted with EtOAc. Organic phase was dried over Na₂SO₃ and concentrated. Crude product was suspended in 4 M HCl in dioxane and stirred for 2 hours. The mixture was concentrated, redissolved in acidic water (0.1% TFA) and washed with CH₂Cl₂. Product was purified on a JASCO High-Performance Liquid Chromatography (HPLC) instrument on a C18 column using aqueous (H₂O+0.1% TFA) and organic (CH₃CN) phases. ¹H NMR (500 MHz, D₂O) δ 7.77 (s, 3H), 7.48 (d, 3H), 7.43-7.41 (m, 3H), 5.77 (s, 1H), 5.73 (s, 1H), 3.42-3.39 (t, 6H), 3.13-3.10 (m, 6H), 2.82 (s, 18H), 2.00-1.94 (m, 6H); ¹³C NMR (500 MHz, D₂O) δ 170.22, 147.81, 144.91, 130.92, 125.1, 124.26, 122.52, 117.47, 55.26, 42.63, 36.48, 24.16; MALDI m/z calcd for C₃₈H₅₀N₆O₃ [M+H] 639.3946, observed 639.326.

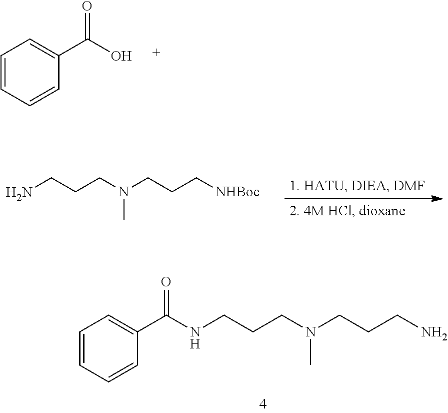 [SMILES: O=C(O)c1ccccc1 ; CN(CCCN)CCCNC(=O)OC(C)(C)C ; CN(CCCN)CCCNC(=O)c1ccccc1]

### N-(3-((3-aminopropyl)(methyl)amino)propyl)benzamide (4)

To a solution of benzoic acid (10 mg, 0.082 mmol) in DMF (0.2 mL) was added HATU (37.4 mg, 0.098 mmol) and DIEA (23.3 mg, 0.18 mmol) and was stirred at room temperature for 5 min. Tert-butyl (3-((3-aminopropyl)(methyl)amino)propyl)carbamate (24.1 mg, 0.098 mmol) was added and stirred overnight. The reaction mixture was concentrated under vacuum, then diluted with water and extracted with EtOAc. Organic phase was dried over Na₂SO₃ and concentrated. Crude product was suspended in 4 M HCl in dioxane and stirred for 2 hours. The mixture was concentrated, redissolved in acidic water (0.1% TFA) and washed with CH₂Cl₂. Product was purified on a JASCO High-Performance Liquid Chromatography (HPLC) instrument on a C18 column using aqueous (H₂O+0.1% TFA) and organic (CH₃CN) phases. ¹H NMR (500 MHz, D₂O) δ 7.83-7.82 (d, 2H), 7.70-7.67 (m, 1H), 7.61-7.58 (m, 2H), 3.58-3.56 (m, 2H), 3.40-3.37 (m, 2H), 3.29-3.27 (m, 2H), 3.16-3.13 (t, 2H), 2.97 (s, 3H), 2.20-2.13 (m, 4H); ¹³C NMR (500 MHz, CDCl₃) δ 171.35, 133.29, 132.38, 128.89, 127.05, 54.03, 52.97, 39.68, 36.57, 36.48, 23.94, 21.94. HRMS m/z calcd for C₁₄H₂₃N₃O [M+H] 250.184, observed 250.1923

HPLC:

HPLC chromatograms were obtained at all wavelengths from 200 to 800 nm. 254 and 214 nm were chosen as virtual channels to show the absorbances at those two specific wavelengths. The blue line corresponds to 254 nm and the red line corresponds to 214 nm (top plot). The lamps used were D2+W with a slit width of 4 nm. A flow rate of 1.00 mL/min was used over 30 minutes. The method began at 10% acetonitrile and 90% water+0.1% TFA. The gradient was slowly increased to 100% acetonitrile.

Melting Temperature Analysis:

An aqueous solution of 10 mM potassium cacodylate (CacoK), pH 7.2 was used as analysis buffer. DNA 3WJ and dsDNA were brought to a final concentration of 1 1.1.1\4 and 2 μM, respectively. Samples were heated to 90° C. for 5 min, cooled to room temperature slowly, then to 4° C. Samples were incubated for 1 hour at room temperature with 3 μL of ligand at a final concentration of 4 μM for DNA 3WJ. The concentration of the ligand was 8 μM for dsDNA. Denaturation was recorded at 260 nm from 5° C. to 90° C. with a heating rate of 0.5° C. min⁻¹. Upper and lower baselines were used to plot the fraction folded.

Circular Dichroism:

DNA was suspended at 20 μM in 10 mM CacoK, pH 7.2 and annealed by heating to 90° C. for 5 min, cooled to room temperature slowly, then to 4° C. Spectra were measured every 0.5 nm between 350 nm and 190 nm with a 5 s averaging time. Samples were incubated at each temperature for 20 minutes prior to scan.

A 50 μM solution of DNA was prepared in 10 mM CacoK, pH 7.2 and annealed by heating to 90° C. for 5 min, cooled to room temperature slowly, then to 4° C., for CD melting. Samples were incubated with ligand (200 μM) at room temperature for 1 hour. The CD melting experiment was measured at 255 nm using a 1° C. step and a 2 min equilibration time. The averaging time was 15 s. All CD spectra were buffer corrected and converted to molar ellipticity.

Fluorescence Quenching Experiments:

All binding experiments were conducted in 50 mM sodium phosphate buffer, pH 7.0. Fluorescence measurements were recorded with excitation at 495 nm and emission at 520 nm using 5 nm bandwidths. Inhibitor strand binding curves were obtained by adding 1 μL of increasing concentrations of inhibitor strand to 19 μL of 120 nM aptamer. Samples were incubated for 1 hour and ran in triplicate in a 384-well plate. Inhibitor strand displacement curves were obtained by incubating 120 nM aptamer with 1 μM inhibitor 12 for 1 hour, followed by addition of increasing concentrations of 1. Samples were incubated for 1 hour and measured in triplicate.

Gel Shift Assay:

The inhibitor strand titration gel was run by incubating aptamer (0.25 μM) with increasing concentrations of inhibitor strand in a 20 μL solution in 50 mM sodium phosphate buffer, pH 7.0 at room temperature for 1 hour. Samples for compound titration were prepared by incubating aptamer (0.25 μM) with inhibitor strand (0.25 μM) for 1 hour followed by titration of 1 and incubated at room temperature for 1 hour. Samples were run on a 15% non-denaturing polyacrylamide gel (19:1 monomer:bis) at 50V in 1×TBE buffer at 4° C. for 5 hours. Gels were stained with SYBR Gold for 10 minutes and visualized using a BioRad GelDoc XR+ imager.

Cell Culture and Cytotoxicity:

All cell lines were maintained in a humidified incubator at 37° C. in 5% CO2. A2780 and A2780cis cells were cultured in RPMI 1640 (Corning Cellgro) supplemented with 10% fetal bovine serum (Giboc, Life Technologies), 1% L-glutamine (Corning Cellgro), penicillin and streptomycin (Corning Cellgro). Cells were seeded at a density of 2,000 cells/well in culture media (50 pt) in 385-well plates 24 hours prior to treatment. Cells were treated with DMSO (vehicle control), doxorubicin (positive control, 10 μM final), cisplatin (50 μM to 25 nM), or triptycenes (50 μM to 25 nM) for 72 hours at 37° C. with 5% CO2. DMSO concentrations were kept at 1%. Alamar blue (5 μL) was added and incubated for 4 hours. Fluorescence was measured at 590 nm. The vehicle control was taken as 100% cell viability and doxorubicin was taken as 0% viability.

Cellular Uptake by MALDI:

A2780 cells were grown as described above. Cells were diluted to 250,000 cells/mL in fresh media. Cells (2 mL) were treated with DMSO (vehicle control) or triptycenes (50 μM final) for 2 hours at 37° C. with 5% CO2. DMSO concentrations were kept at 1%. The cells were centrifuged at 640 g for 2 minutes. The supernatant was removed and the cells were washed with 500 μL of 50 mM Tris-HCl, pH 7.4 three times. The supernatant was placed in a clean microcentrifuge tube for MALDI analysis. The cell pellet was suspended in lysis buffer (0.3% Triton-X-100, 100 mM NaCl) and heated to 100° C. for 15 minutes. The lysate was centrifuged at 7080 g for 5 minutes, the supernant was transferred to a clean microcentrifuge tube. MALDI was used to analyze the washes and lysate using a-cyano-4-hydroxycinnamic acid as the matrix.

### Example 2

Modulation of the rpoH Temperature Sensor in E. coli

Figures and references are as published in Barros et al., Angew. Chem. Int. Ed., 2016(55):8258, published on May 30, 2016.

Regulation of the heat shock response (HSR) is essential in all living systems. In E. coli, the HSR is regulated by an alternative σ factor, σ³², which is encoded by the rpoH gene. The mRNA of rpoH adopts a complex secondary structure that is critical for the proper translation of the σ³² protein. At low temperatures, the rpoH gene transcript forms a highly structured mRNA containing several three-way junctions, including a rare perfectly paired three-way junction (3WJ). This complex secondary structure serves as a primitive but highly effective strategy for the thermal control of gene expression. In this work, the first small-molecule modulators of the E. coli σ³² mRNA temperature sensor are reported.

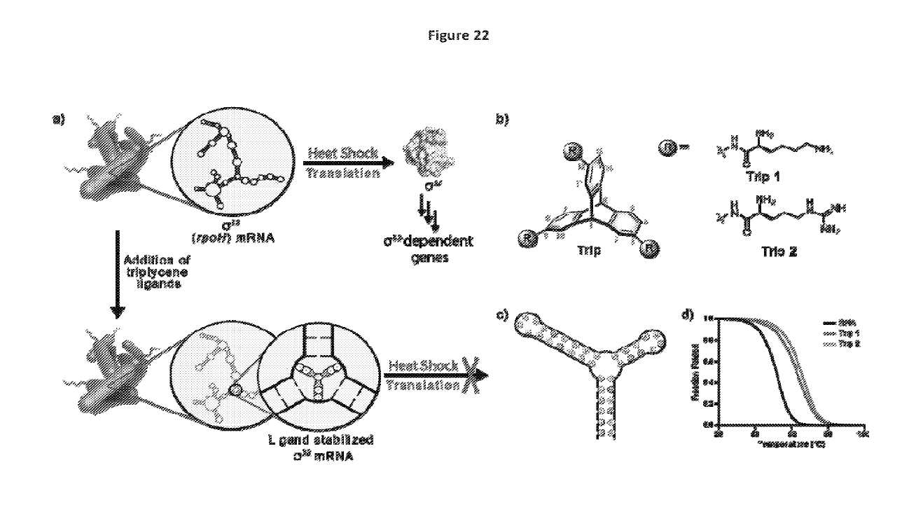

Temperature is a universal stress factor for all living organisms, and a rapid response to temperature fluctuations is essential for cell survival. The heat shock response (HSR) is a cellular process characterized by the increased synthesis of a set of heat shock proteins (HSPs) in response to stress, such as temperature. The Escherichia coli (E. coli) HSR is regulated by an alternative σ factor, σ³², encoded by the rpoH gene. An increase in temperature from 30° C. to ≥37° C. results in the increased synthesis and stability of σ³², leading to the transcription of σ³²-dependent genes involved in the HSR. Translational control is a common strategy for the modulation of the HSR in both eukaryotes and prokaryotes. It is found that the σ³² mRNA secondary structure acts as a thermosensor, crucial for the induction of σ³², in the E. coli HSR pathway (FIG. 22). Intramolecular base-pairing interactions in the first 229 nucleotides control the translation efficiency of σ³². Analysis of a series of deletions and mutations shows the presence of two regulatory elements that fold into a complex structure, preventing the initiation of translation at low temperatures. The first regulatory element is a 15 nucleotide downstream box (region A) near the AUG start codon that allows for binding of the 30S ribosome. The second regulatory element, stem III (FIG. 22b), blocks the downstream box. The AUG start codon is then blocked by nucleotides present in stem I. Base pairing of the start codon and the downstream box by stems I and III prevents ribosome binding at low temperatures. Primer-extension inhibition (toeprinting) experiments have demonstrated that thermal stress disrupts the RNA secondary structure, leading to ribosome binding and increased translation. These experiments directly correlated the degree of ribosome binding to RNA stability. Very few small molecules have been developed for direct prokaryotic or eukaryotic translational control at the RNA level. Small-molecule probes with the ability to stabilize the σ³² mRNA secondary structure could be useful probes for studying the HSR pathway as well as potential antibacterial agents or adjuvants.

Chemical and enzymatic probing of the 5′-end of the σ³² mRNA secondary structure reveals that the regulatory regions (regions A and B) within the RNA structure form a perfectly paired three-way junction (3WJ). Recently, we developed a new class of nucleic acid junction binders based on the triptycene scaffold. Herein, we report the first triptycene-based small molecules that are able to modulate the stability of the σ³² mRNA. We determined the ability of these ligands to modulate the structure of σ³² RNA by UV thermal melting, circular dichroism (CD), and fluorescence quenching experiments. Furthermore, we demonstrate the in vivo modulation of the heat shock response using a σ³²-GFP fusion protein reporter system in E. coli.

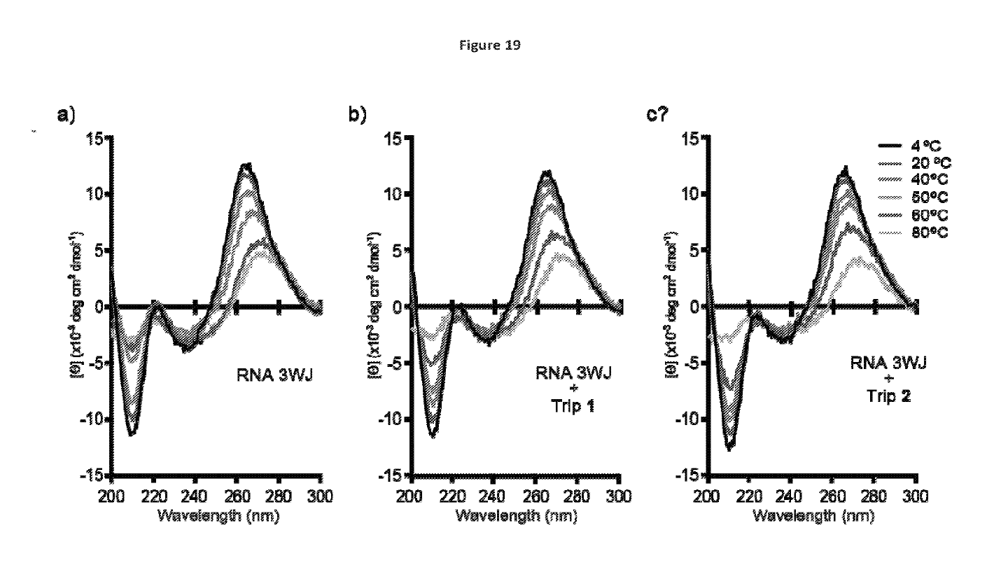

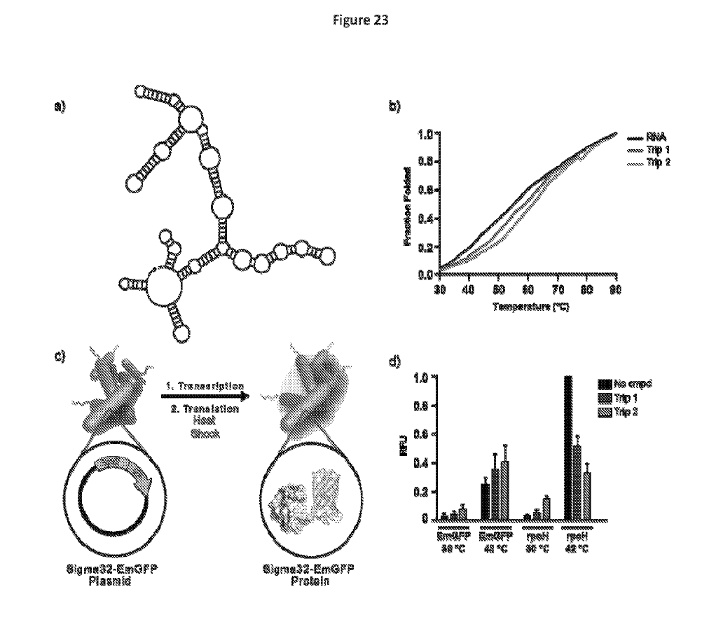

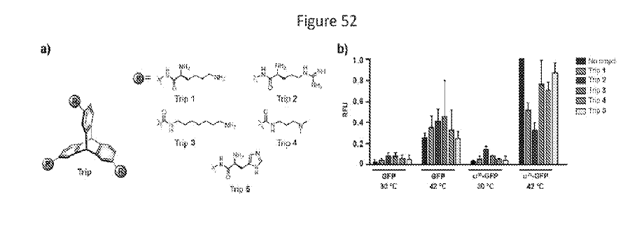

We initiated our studies with a model system corresponding to the regulatory junction present in the rpoH mRNA (FIG. 23). UV melting experiments were performed to determine the ability of Trip 1 and 2 (FIG. 23a, c) to stabilize the model system. In the absence of ligand, the RNA melted at 51.6° C. Thermal stabilization was observed in the presence of Trip 1 and 2, with ΔTm values of 11.3 and 13.7° C., respectively (FIG. 23c). CD spectroscopy was also performed to investigate the interaction of Trip 1 and 2 with the RNA. CD spectra of the model system in the presence and absence of Trip 1 and 2 at 48 C are consistent with A-form RNA, displaying a maximum at 266 nm, a large minimum at 210 nm, and a smaller minimum around 240 nm (FIG. 19). As the temperature increases from 4° C. to 80° C., the maximum at 266 nm decreased and the minimum at 210 nm became less negative. These changes are indicative of the melting of the helical segments. Temperature-dependent CD spectroscopy in the presence of Trip 1 and Trip 2 gave the same trend, but the change was more gradual, particularly between 50° C. and 80° C. (FIG. 19). This is consistent with ligand-induced stabilization as observed in the UV experiment. CD spectra in the presence of increasing concentrations of the triptycenes show slight signal changes (FIG. 52). A more negative signal is observed at 210 nm as well as a decrease and slight shift at 220 nm. These changes are not consistent with intercalation or groove-binding modes, rather they are suggestive of native helical structural stabilization through a non-helix-perturbing binding event.

A fluorescence quenching experiment was used to further support the modulation of the σ³² RNA (FIG. 23d). The oligonucleotide was labeled with a fluorophore on the 5′-end and a quencher on the 3′-end. Once the RNA is folded, little to no fluorescence is observed as the fluorophore and quencher are in close proximity. Upon addition of a 16 base pair oligonucleotide complementary to the 5′-end, an increase in fluorescence is observed, indicating that both ends are further apart in space owing to the formation of an open state (FIG. 23e). The addition of Trip 1 and 2 to this open-state structure results in a decrease in fluorescence, which is consistent with reformation of the folded 3WJ state (FIG. 23f). The apparent K_{d} values of Trip 1 and Trip 2 were determined to be 2.5 mm and 1.5 mm, respectively.

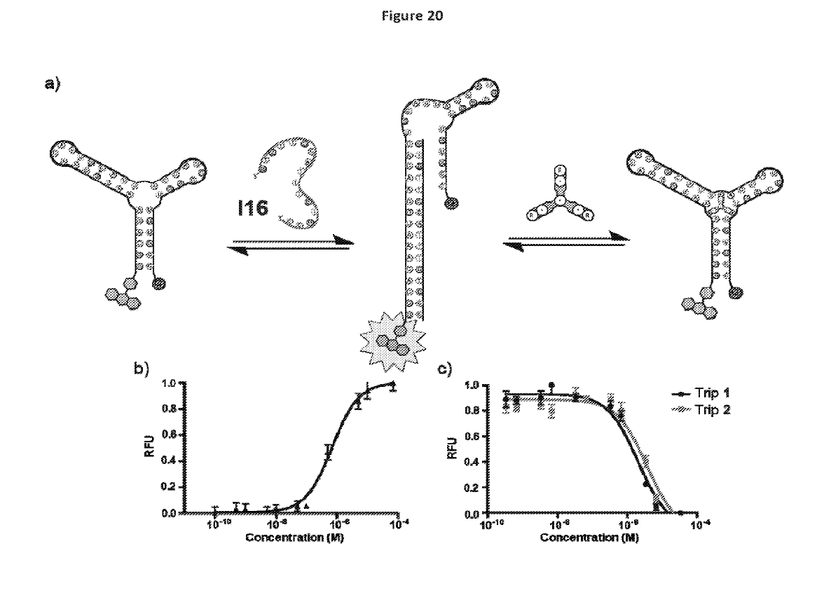

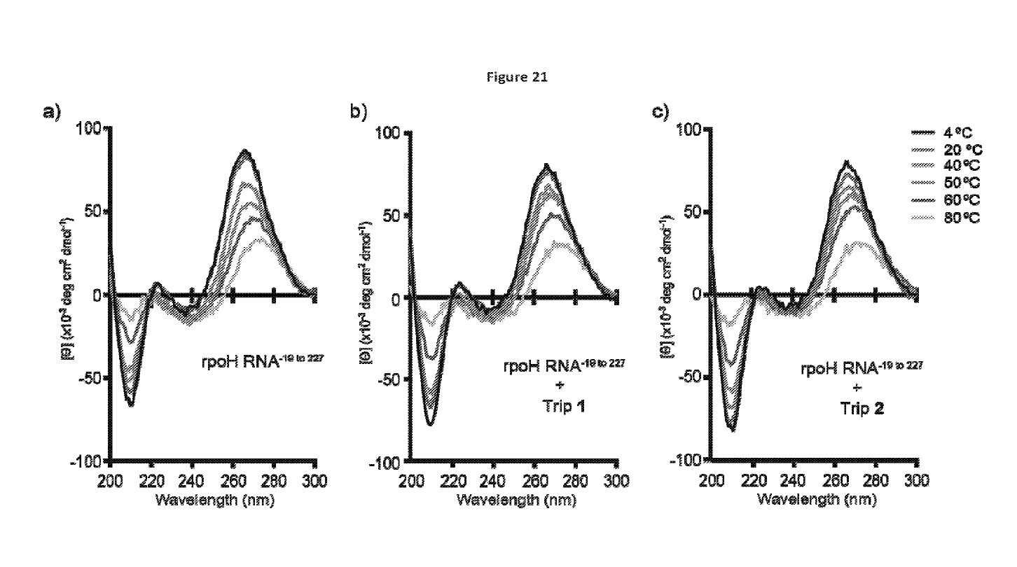

Having characterized the interactions of Trip 1 and 2 with the model system by fluorescence quenching, we turned our attention to the full 5′-region of the σ³² mRNA (−19 to +229). The 5′-end of the σ³² mRNA was transcribed in vitro for characterization by temperature-dependent UV and CD techniques. UV thermal melting experiments in the absence of the triptycenes showed a double inflection, indicating that portions of the RNAmelt at different temperatures, with the most critical structural changes occurring below 42° C. The first inflection, with an initial onset below 42° C., is consistent with the temperature-dependent translation previously observed for rpoH mRNA. In the presence of Trip 1 and Trip 2, thermal stabilization of the full-length σ³² mRNA (−19 to +229, FIG. 20a) is observed. The shift in thermal stabilization is especially prominent between 30 and 60° C. The CD spectra in the absence and presence of Trip 1 and 2 are consistent with an A-form RNA structure (FIG. 21). The melting of the helical regions of the RNA was confirmed by temperature-dependent CD spectroscopy. The addition of Trip 1 or 2 resulted in a more negative peak at 210 nm and a slightly lower maximum at 266 nm, which is indicative of a structural change in the RNA. Thermal stabilization is maximally observed between 40° C. and 50° C. and between 60° C. and 80° C. in the presence of the triptycenes.

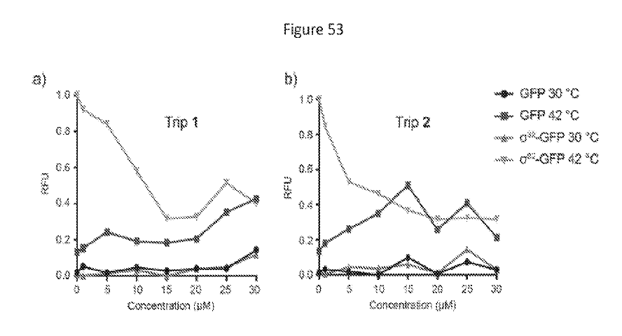

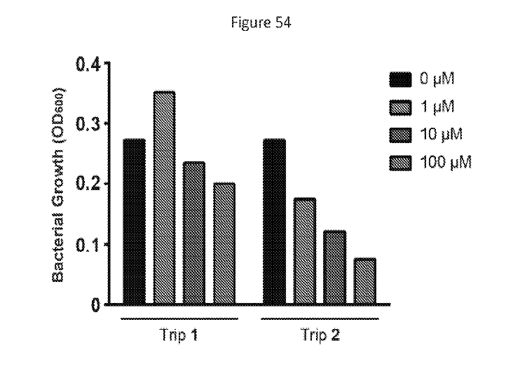

A reporter assay based on a σ³² GFP fusion protein was developed and used to monitor the responses to cellular stress in E. coli (FIG. 20b). The rpoH gene, which codes for the σ³² protein, along with its promoters, was PCR-amplified from the genomic DNA of E. coli and inserted into a plasmid encoding GFP. Cells were grown at 30° C. for several hours in the presence or absence of various tricptycene derivatives, followed by heat shock at 42° C. (FIG. 52). Cells containing the control GFP plasmid (no σ³²) showed low relative GFP fluorescence when grown at 30° C. (FIG. 3c). An increase in temperature to 42° C. resulted in a slight increase in fluorescence in the absence and presence of triptycene derivatives using the GFP control plasmid. As expected, cells that contained the σ³²-GFP fusion protein and were grown at 30° C. showed low fluorescence similar to the GFP control plasmid. However, those grown at 42° C. displayed a large increase in GFP fluorescence in the absence of these compounds. Upon triptycene addition, a decrease in fluorescence was observed to varying degrees at 42° C. The most significant decrease in fluorescence was observed in the presence of Trip 1 or Trip 2 compared to Trip 3, 4, and 5 (FIG. 52). The addition of a 5 mm solution of Trip 1 or Trip 2 resulted in a slight decrease in the relative fluorescence intensity at 42° C. This decrease was more significant at triptycene concentrations of 25 mm. The relative fluorescence intensities were similar to those observed with the control GFP plasmid, indicating a loss of the heat shock response in the presence of both triptycenes, although Trip 2 appeared to be more potent. A concentration-dependent decrease in the signal at 42° C. was also observed (FIG. 53). This is consistent with thermal stabilization of the mRNA by the triptycenes, where upon an increase in temperature, the structure is more folded and stable, suppressing translation of the σ³²-GFP fusion protein. The increased translational inhibition with Trip 2 over Trip 1 could be due to a combination of affinity, cell permeability, or various non-specific interactions.

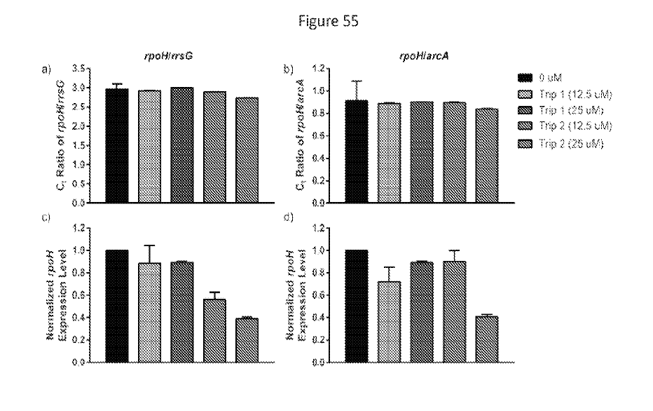

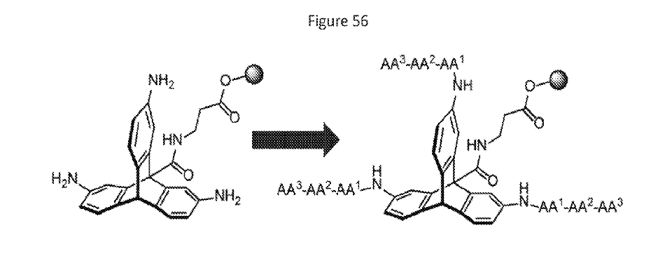

Non-specific inhibition of translation was evaluated using a control GFP-only plasmid in the presence of Trip 1 and Trip 2. Interestingly, we observed an approximately fourfold increase in translation going from the GFP control plasmid at 42° C. to the σ³²-GFP plasmid at 42° C. (FIG. 3c). This is reflective of increased translation upon incorporation of the heat-shock-responsive σ³² RNA element, which promotes translation at higher temperatures. Furthermore, we observed a mild increase in translation upon treatment with Trip 1 and 2, except in the case of σ³²-GFP at 42° C. Polyamines have been shown to enhance translation in certain cases, and this could be the origin, although the effect is small. Bacterial growth experiments in the presence of the triptycenes indicate that Trip 1 and Trip 2 are moderately inhibitory at high concentrations (FIG. 54). Furthermore, we conducted qRT-PCR experiments to gauge the amount of transcriptional inhibition induced by Trip 1 and Trip 2. Overall, the mRNA levels of σ³² were not affected by Trip 1 and only moderately affected at high concentrations of Trip 2, indicating little inhibition of transcription or differential mRNA stabilization, except with high levels of Trip 2 (FIG. 55).

In summary, we have described triptycene-based molecules that modulate the 5′-region of the σ³² mRNA temperature sensor from E. coli. Trip 1 and Trip 2 thermally stabilize a model system consisting of the critical central three-way junction that is present in the σ³² mRNA and responsible for regulation of the heat shock response as determined by UV thermal melting experiments and temperature-dependent CD spectroscopy. UV thermal melting experiments on the full 5′-region of the σ³² mRNA also show thermal stabilization. This stabilization was corroborated by temperature-dependent CD spectroscopy in the presence of ligands. To determine the effect of the triptycenes on the heat shock response in E. coli, a σ³²-GFP fusion protein assay was utilized. In the absence of the triptycenes, an increase in fluorescence was observed when the cells were heat-shocked at 42° C., indicating σ³² protein translation. However, the addition of Trip 1 or Trip 2 suppresses the fluorescence, which is consistent with a decrease in σ³² protein translation. This new class of small molecules may be useful for studying the effects of the heat shock response in E. coli. Furthermore, modulation of the temperature-sensing RNA regulatory elements in bacteria could lead to the development of novel methods for targeting pathogens or potentiating current antibiotics.

General Methods:

The σ³² mRNA model system (5′-GGCACAAACGCAACACUGCAUUACCAUGCGGUUGUGCC-3′), Inhibitor 16 (I16) (5′-GTGTTGCATTTGTGCC-3′) and all other oligonucleotides were purchased from Integrated DNA Technologies (IDT). HPLC-purified σ³² mRNA model system modified with a 5′-FAM and a 3′-IowaBlack was also purchased from IDT. E. coli genomic DNA was purchased from Addgene (Cambridge, Mass. USA). Restriction enzymes and T4 DNA ligase were purchased from New England BioLabs. T7 RNA polymerase was purchased from Promega (Madison, Wis. USA). Milli-Q (18 MS2) water was used for all solutions (Millipore; Billerica, Mass., USA).

Ultraviolet absorption spectroscopy and thermal denaturation experiments were performed on a JASCO V-650 spectrophotometer (JASCO Analytical Instruments; Easton, Md., USA) equipped with a JASCO PC-734R multichannel Peltier using quartz cells with 1 cm path lengths. Circular dichroism experiments were performed on a JASCO J-1500 CD Spectrometer (Easton, Md., USA) using a 0.1 cm path length quartz cuvette. Fluorescence measurements were collected on a Tecan M1000 plate reader (Mannedorf, Switzerland). Polymerase chain reaction (PCR) was run on a BioRad C1000 Touch Thermocycler.

Synthesis:

Trip 1 and Trip 2 were synthesized according to previously described methods, S. A. Barros, D. M. Chenoweth, Chem. Sci. 2015, 6, 4752-4755, hereby expressly incorporated herein by reference for the methods, figures and legends herein.

Synthesis of rpoH mRNA −19 to +229:

Plasmid pRSET-EmGFP (HA EmGFP ABC2 V94F) was used for cloning into the XbaI and EcoRI restriction sites. The rpoH (−19 to 229) gene was obtained by PCR amplification from genomic DNA from E. coli K-12. The forward and reverse primers used were 5′-GATCTAGAATCGATTGAGAGGATTTGAATG-3′ and 5′-GAGAATTCCCGCCTGTGGCAGGCCATAGC-3′, respectively. The pRSET-EmGFP plasmid was digested with XbaI and EcoRI then gel purified to isolate the linear vector. The σ³² (−19 to 229) PCR product was also digested and inserted into the plasmid using T4 DNA ligase. The resulting plasmid was verified by DNA sequencing using a T7 primer.

The DNA template was prepared for transcription by linearization with EcoRI then gel purified. The RNA was transcribed in vitro by T7 RNA polymerase (Promega). In vitro transcription reactions were set up using the protocol supplied by Promega with 1× transcription buffer, 10 mM DTT, 0.5 mM each rNTP, and 2-5 μg DNA.

UV Thermal Denaturation: model system RNA was suspended at 1 μM in 10 mM sodium phosphate buffer, pH 7.2 and annealed by heating to 90° C. for 5 min, cooled to room temperature slowly, then to 4° C. Samples were incubated for 1 hour at room temperature with 1 μL of ligand at a final concentration of 2 μM. σ³² RNA (−19 to +229) was suspended at 0.25 μM in 10 mM sodium phosphate buffer, pH 7.2 and annealed by heating to 65° C. for 5 min, cooled to room temperature slowly, then to 4° C. Samples were incubated for 1 hour at room temperature with 1 μL of ligand at a final concentration of 2.5 μM. Denaturation was recorded at 260 nm from 20° C. to 90° C. with a heating rate of 0.5° C. min-1.

Fluorescence quenching experiments: all binding experiments were conducted in 50 mM sodium phosphate buffer, pH 7.2. Fluorescence measurements were recorded with excitation at 495 nm and emission at 520 nm using 5 nm bandwidths. Inhibitor strand binding curves were obtained by adding 1 μL of increasing concentrations of inhibitor strand to 19 μL of 120 nM RNA. Samples were incubated for 2 hours and ran in triplicate in a 384-well plate. Inhibitor strand displacement curves were obtained by incubating 120 nM RNA with 1.4 μM inhibitor 16 for 2 hours, followed by addition of increasing concentrations of Trip 1 or Trip 2. Samples were incubated for 2 hours and measured in triplicate.

Circular Dichroism:

Model system RNA was suspended at 5 μM in 10 mM sodium phosphate buffer, pH 7.2 and annealed by heating to 90° C. for 5 min, cooled to room temperature slowly, then to 4° C. Spectra were measured every 1 nm between 350 nm and 200 nm with a 16 s averaging time. Samples containing ligand were incubated with Trip 1 or Trip 2 (10 μM) at room temperature for 1 hour. Samples were incubated at each temperature for 20 minutes prior to scan. All CD spectra were buffer corrected and converted to molar ellipticity.

σ32 mRNA (−19 to +229) was suspended at 0.5 μM in 10 mM sodium phosphate buffer, pH 7.2 and annealed by heating to 65° C. for 5 min, cooled to room temperature slowly, then to 4° C. Spectra were measured every 1 nm between 350 nm and 200 nm with a 16 s averaging time. Samples containing ligand were incubated with Trip 1 or Trip 2 (5 μM) at room temperature for 1 hour. Samples were incubated at each temperature for 20 minutes prior to scan All CD spectra were buffer corrected and converted to molar ellipticity.

σ³²-EmGFP Plasmid Construction: Plasmid pRSET-EmGFP (HA EmGFP ABC2 V94F) was used for cloning into the XbaI and EcoRI restriction sites. The rpoH gene was obtained by PCR amplification from genomic DNA from E. coli K-12. This included four rpoH promoters (p2, p3, p4, and p5). The forward and reverse primers used were 5′-GATCTAGAGAACTTGTGGATAAAATCACG-3′ and 5′-GAGAATTCGGATCCTTACGCTTCAATGGCAGCAC-3′, respectively. The pRSET-EmGFP plasmid was digested with XbaI and EcoRI then gel purified to isolate the linear vector. The rpoH PCR product was also digested and inserted into the plasmid using T4 DNA ligase. The resulting plasmid was verified by DNA sequencing using a T7 primer.

σ³²-EmGFP Assay: E. coli DHSa cells transformed with the 632-EmGFP plasmid were grown overnight at 30° C. in Luria broth (LB) supplemented with 50 μg/mL ampicillin. Overnight cultures were diluted 1:100 in LB. Triptycenes were added at a final concentration of 25 μM. Samples were allowed to grow at 30° C. for 3 hours. Cultures were kept at 30° C. or heat shocked at 42° C. for 18 hours. Optical density was measured at 600 nm. Fluorescence was measured by excitation at 486 nm and emission at 535 nm. Measurements were made in triplicate for each sample. The raw fluorescence intensity was divided by the optical density at 600 nm, which was then normalized.

qRT-PCR Experiment: E. coli DH5a cells transformed with the 632-EmGFP plasmid were grown as described in σ³²-EmGFP Assay. Triptycenes were added at a final concentration of 25 or 12.5 μM. Samples were allowed to grow at 30° C. for 3 hours. Cultures were then kept at 30° C. for 18 hours. Total RNA from E. coli was extracted and purified using RNAprotect Bacteria Reagent (QIAGEN, catalog #: 76506) and RNeasy Mini Kit (QIAGEN, catalog #: 74104). The user manual provided by QIAGEN was followed. The expression was quantified in quadruplicate by qRT-PCR using Custom TaqMan™ Gene Expression Assays (Applied Biosystems by Life Technologies, Foster City, Calif., USA) at the University of Pennsylvania Perelman School of Medicine Molecular Profiling Core. The reverse transcription reaction was carried out with High Capacity cDNA Reverse Transcription Kit (Applied Biosystems) in 100 μl containing 1.0 μg RNA in 30.0 μl nuclease free water, 4 μl of 25× (100 mM) dNTPs, 5 μl of multiscribe reverse transcriptase (50 U/μl), 10 μl of 10× reverse transcription buffer, 10 μl 10× random primer. For synthesis of cDNA, the reaction mixtures were incubated at 25° C. for 10 min, at 37° C. for 120 min, at 85° C. for 5 min and then held at 4° C. Then, 4.5 μl of 1:5 diluted cDNA solution was amplified using 5.0 μl TaqMan 2× Fast Universal PCR Master Mix with no AmpErase UNG (Applied Biosystems), 0.5 μl of assay in a final volume of 10.0 μl. Quantitative PCR was run on a QuantStudio 12K Flex Real-Time PCR system (Applied Biosystems) and the reaction mixtures were incubated at 95° C. for 10 min, followed by 40 cycles of 95° C. for 15 s and 60° C. for 1 min. The cycle threshold (Ct) values were calculated with QuanStudio Software version 1.2.2 (Applied Biosystems). The mRNA levels of rpoH were normalized to two housekeeping genes, rrsG (16S ribosomal RNA of rrnG operon) and arcA (response regulator in two-component regulatory system with ArcB or CpxA), using the methods described below.

Method A: Cₜ (rpoH)/Cₜ (rrsG or arcA)

Method B²:

ΔCₜ₁=Cₜ(rpoH_Trip 1 or 2)−Cₜ(rrsG or arcA_Trip 1 or 2)

ΔCₜ₂=Cₜ(rpoH No compound)−Cₜ(rrsG or arcA_No compound)

ΔΔCₜ=Cₜ₁−Cₜ₂

Normalized rpoH gene expression level=2^{ΔΔCt}

### Example 3

### Triptycene-Based Small Molecules Modulate (CAG)⋅(CTG) Repeat Junctions

The figures and references are as published in Barros et al., Chemical Science, 2015 (6):4752, published on Jun. 10, 2015.

Nucleic acid three-way junctions (3WJs) play key roles in biological processes such as nucleic acid replication in addition to being implicated as dynamic transient intermediates in trinucleotide repeat sequences. Structural modulation of specific nucleic acid junctions could allow for control of biological processes and disease states at the nucleic acid level. Trinucleotide repeat expansions are associated with several neurodegenerative diseases where dynamic slippage is thought to occur during replication, forming transient 3WJ intermediates with the complementary strand. Here, we report triptycene-based molecules that bind to a d(CAG)⋅(CTG) repeat using a gel shift assay, fluorescence-quenching and circular dichroism

Nucleic acid junctions play important roles in biological processes and serve as key structural motifs in nanotechnology and aptamer-based sensing applications. In biology, three-way junctions (3WJs) are found as transient intermediates during replication, recombination, and DNA damage repair. Junctions are also present in several viral genomes, such as HIV-1, HCV, and adeno-associated virus in addition to playing key roles in viral assembly. Nucleic acid junctions are also prevalent in the emerging field of DNA and RNA nanotechnology where the bacteriophage phi29 pRNA containing RNA three-way junctions provide a particularly impressive example. Furthermore, they occur in trinucleotide repeat expansions found in unstable genomic DNA associated with neurodegenerative diseases. The development of structure and sequence-specific nucleic acid binding molecules remains an important challenge in chemical biology. The ability to target specific motifs using small molecules would allow for the precise control of biological processes and the possibility of modulating disease states.

DNA trinucleotide repeats are present throughout the genome. Expansions of these repeats, however, are associated with a number of neurodegenerative diseases, including Huntington's disease, spinobulbar muscular atrophy, and mytonic dystrophy. Current models of triplet repeat expansion disease suggest slippage during DNA synthesis by the formation of dynamic DNA hairpin structures. As the length of the repeat increases, the growing hairpin structure gains thermodynamic stability, with repeat length providing an important positively correlated diagnostic for disease severity. Slipped-out (CAG)ₙ⋅(CTG)ₙ repeats have been implicated in the pathogenesis of triplet repeat expansion diseases such as Huntington's disease and several others. These “slipped-out” regions are dynamic and occur along the duplex, forming three-way junctions. Current models suggest that one arm of the junction contains the excess repeats while the other arms maximize complementary pairing between adjacent strands. A dynamic ensemble of conformations are possible at the slipped junction interface, where base pairing interactions differ with each state. The slipped-out (CAG)ₙ repeat in FIG. 4 has been shown to contain one unpaired base at the center of the junction. Previous NMR studies have demonstrated that this sequence can adopt a multitude of conformations, where dynamic single nucleotide bulges at the junction interface interconvert between structures. Small molecule probes could provide important tools for gaining molecular level insight into the dynamics and repair processes associated with trinucleotide repeat junctions; however, probes of this kind are currently unknown.

Recently, we reported a new class of triptycene-based three-way junction (3WJ) binders. Here, we apply the triptycene scaffold as a first step toward developing new tools to recognize trinucleotide repeat junctions. We assessed the ability of our new triptycene based molecules to modulate the structure of d(CAG)ₙ repeats using gel shift assays, a fluorescence quenching assay, and circular dichroism (CD).

Results and Discussion

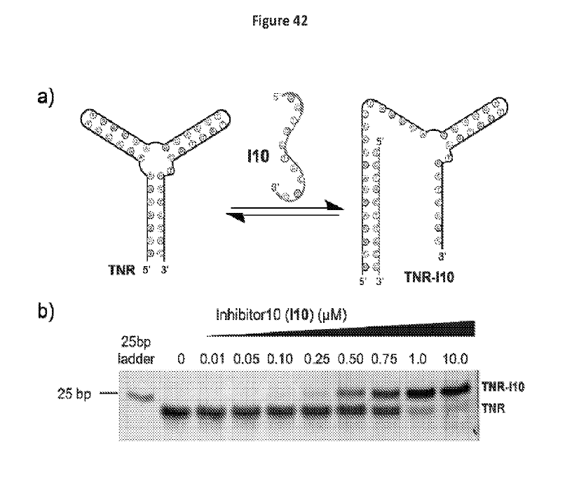

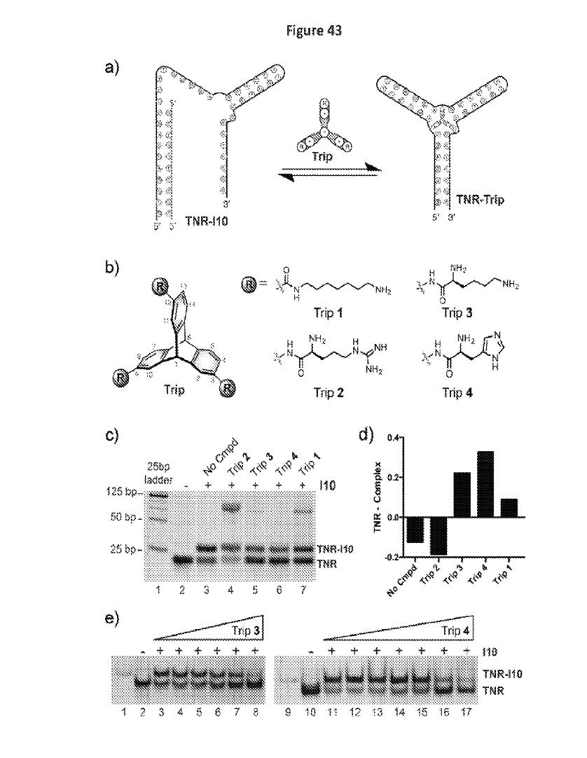

Utilizing a well-studied (CAG)⋅(CTG) repeat sequence known to form slipped-DNA three-way junctions, we developed a competitive inhibitor-based gel shift assay. This assay was inspired by previous work from our laboratory and seminal studies from the Plaxco and Ricci laboratories. We utilized the junction forming (CAG)⋅(CTG) repeat sequence (TNR) and an inhibitor strand (I10) shown in FIG. 42a. The optimal inhibitor strand was 10 base pairs long and complementary to the 5′-end of the junction. Titration of inhibitor strand I10 into the folded junction resulted in a concentration dependent supershift of the band corresponding to hybridization of the TNR junction with I10, forming a larger molecular weight complex (TNR-I10) (FIG. 42b). Addition of small molecules capable of binding the TNR junction leads to HO strand displacement from the TNR-I10 complex in a concentration dependent manner (FIG. 43a).

Inspired by nucleic acid binding proteins, we synthesized a new class of triptycene molecules containing amino acids commonly found at the protein-nucleic acid interface. Analysis of these interfaces reveals an abundance of positively charged amino acid residues such as arginine, lysine, and histidine. We synthesized triptycene derivatives substituted with arginine, lysine, and histidine (Trip 2-4) as shown in FIG. 43b. Compounds 2-4 were assessed using a competitive displacement gel shift assay and compared to Trip 1, which was previously shown to significantly stabilize 3WJs (FIG. 43). Folded TNR 3WJ was incubated with I10, followed by addition of triptycenes 1-4 at the same concentration. Triptycenes 1-4 were able to reform the junction to varying degrees (FIGS. 43c and d). Trip 3 and Trip 4 were the most effective, while Trip 1 was slightly less effective and Trip 2 did not show reformation of the junction. Interestingly, addition of Trip 1 and Trip 2 resulted in a slower moving band on the gel, indicating formation of a higher order structure. Trip 3 and Trip 4 resulted in the most significant inhibitor (I10) displacement, shifting TNR-I10 to the TNR-Trip complex and reforming the junction. A full titration of Trip 3 and Trip 4 with pre-incubated TNR-I10 was then performed (FIG. 43e). Concentration dependent displacement of the inhibitor strand (I10) resulted in reformation of the junction.

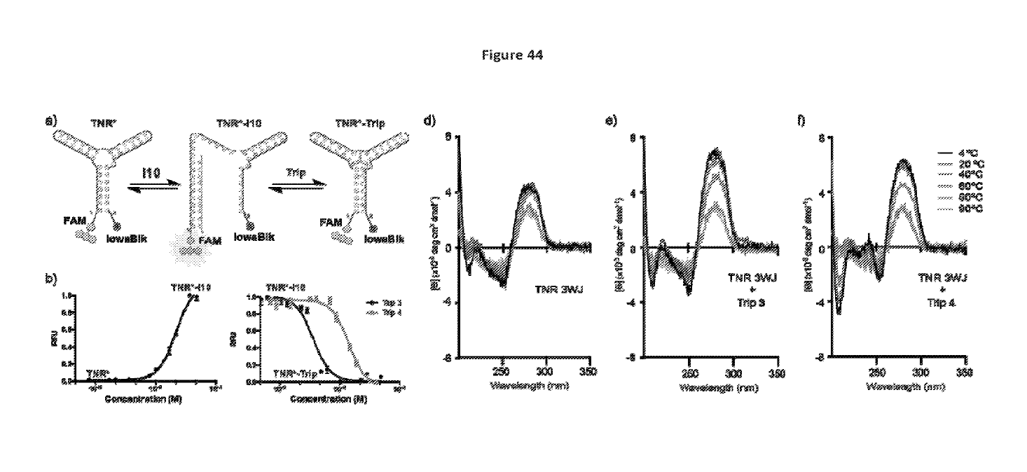

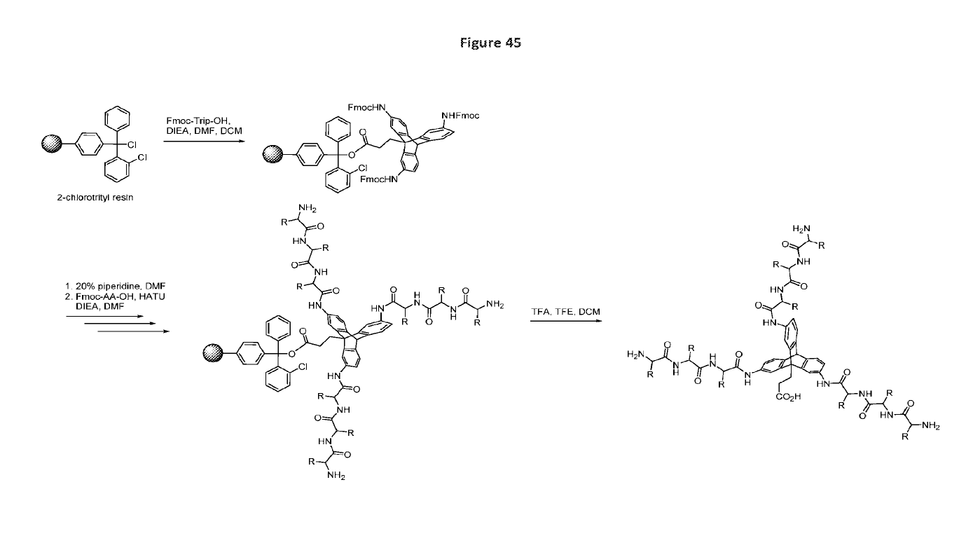

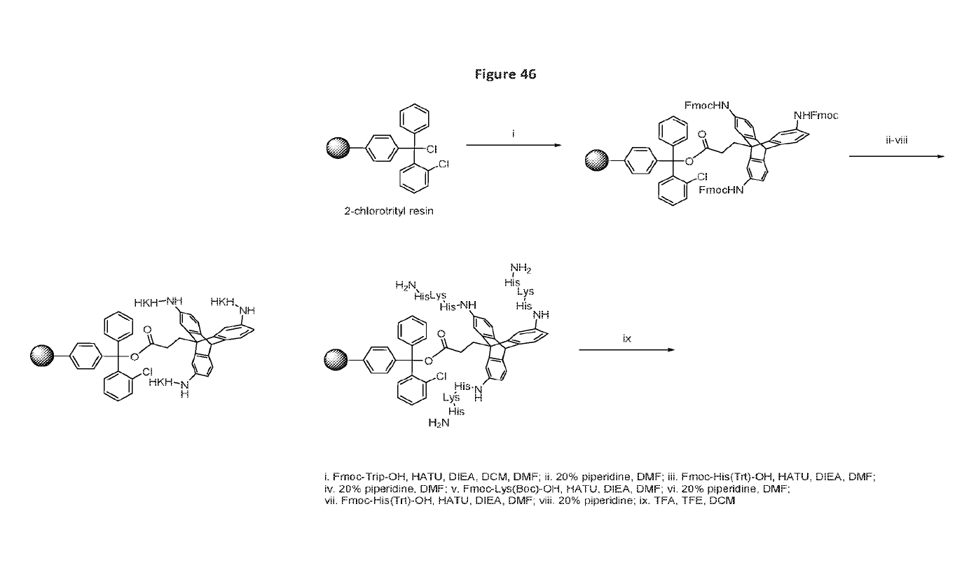

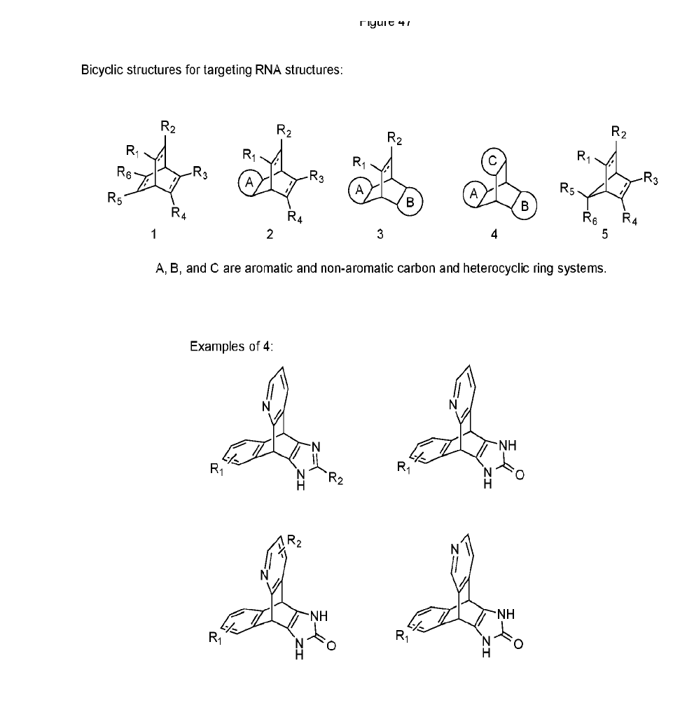

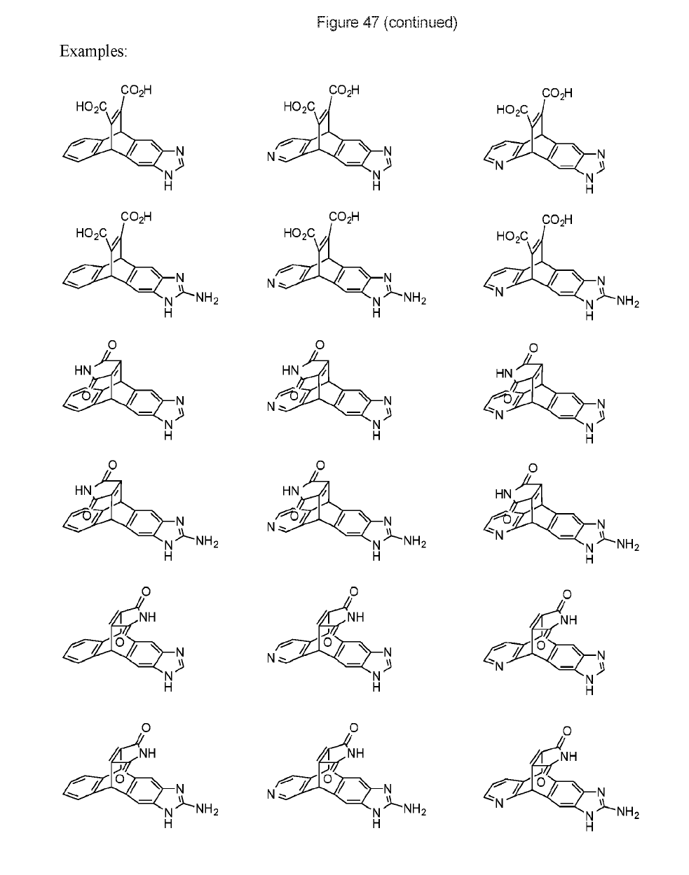

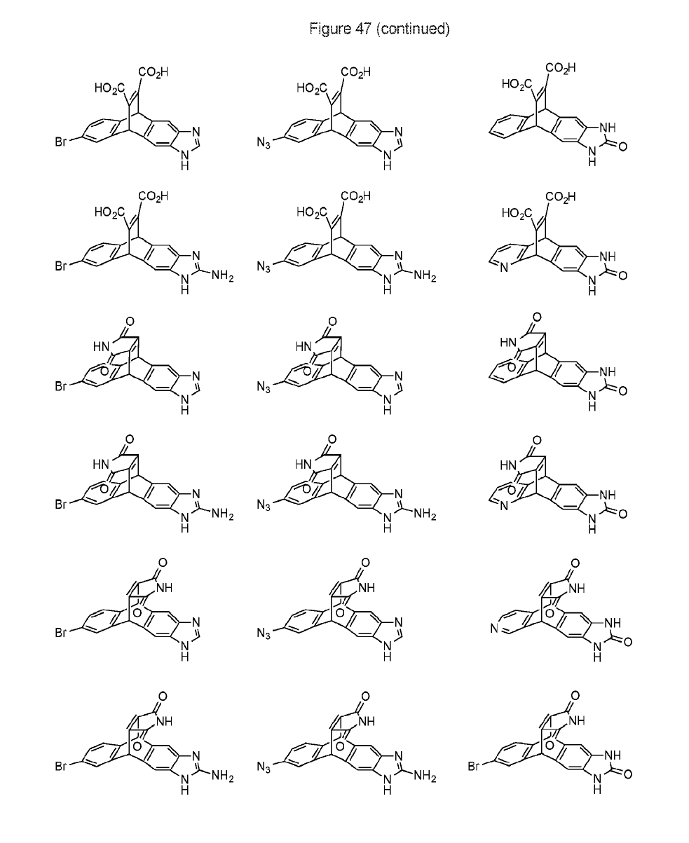

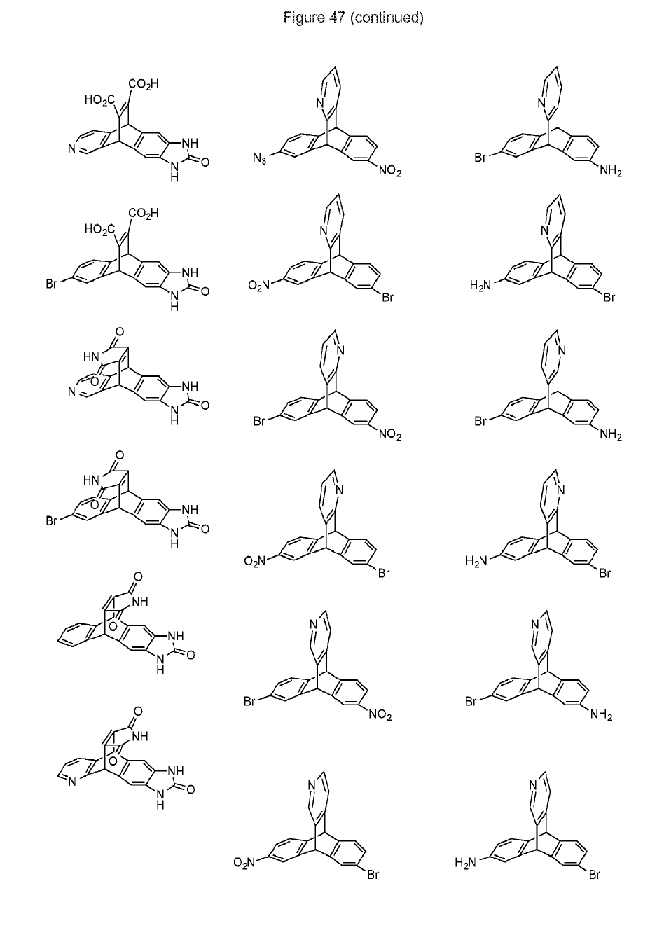

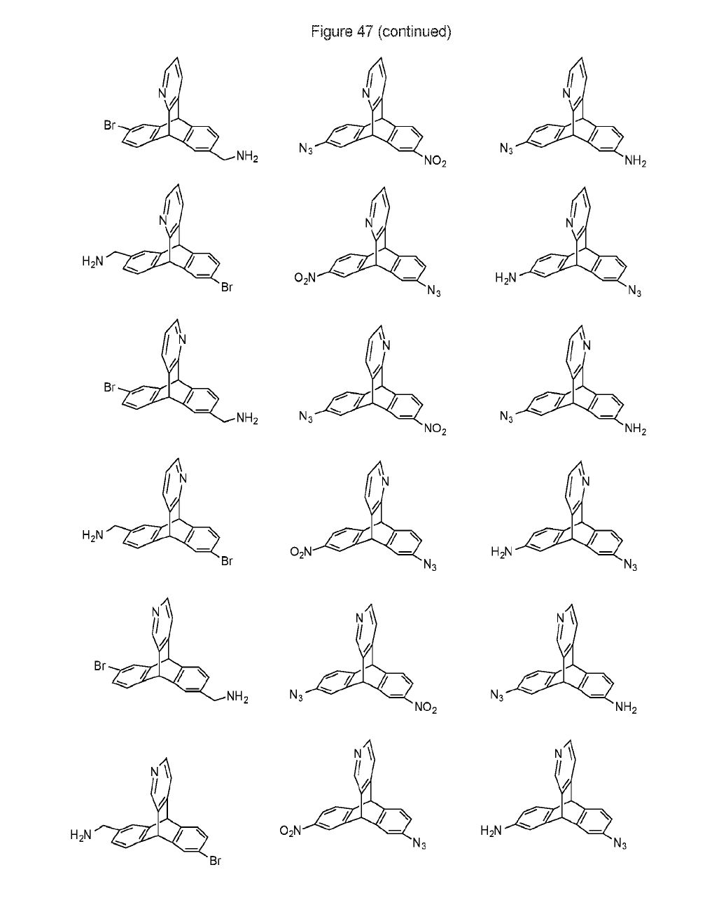

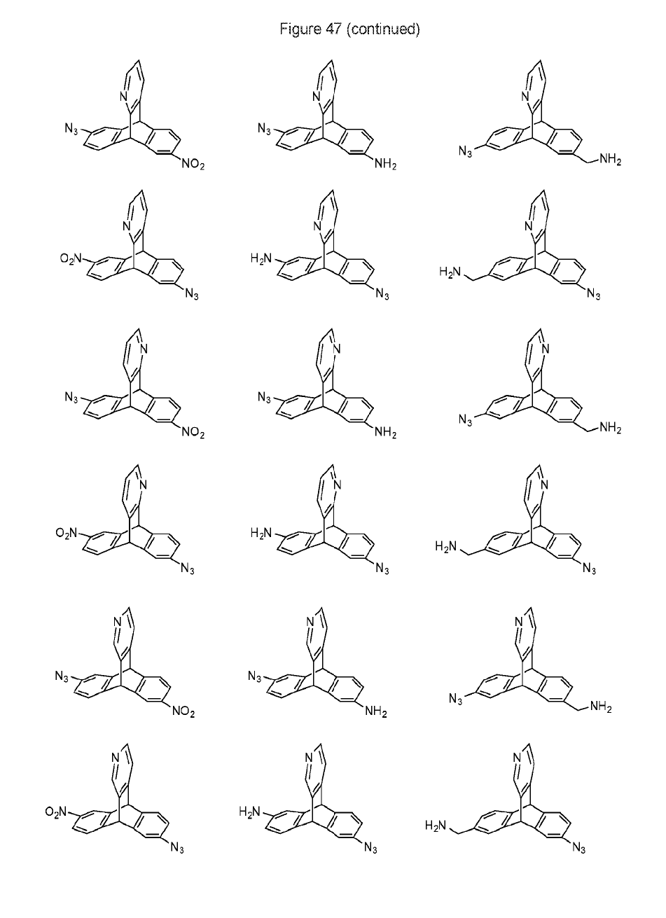

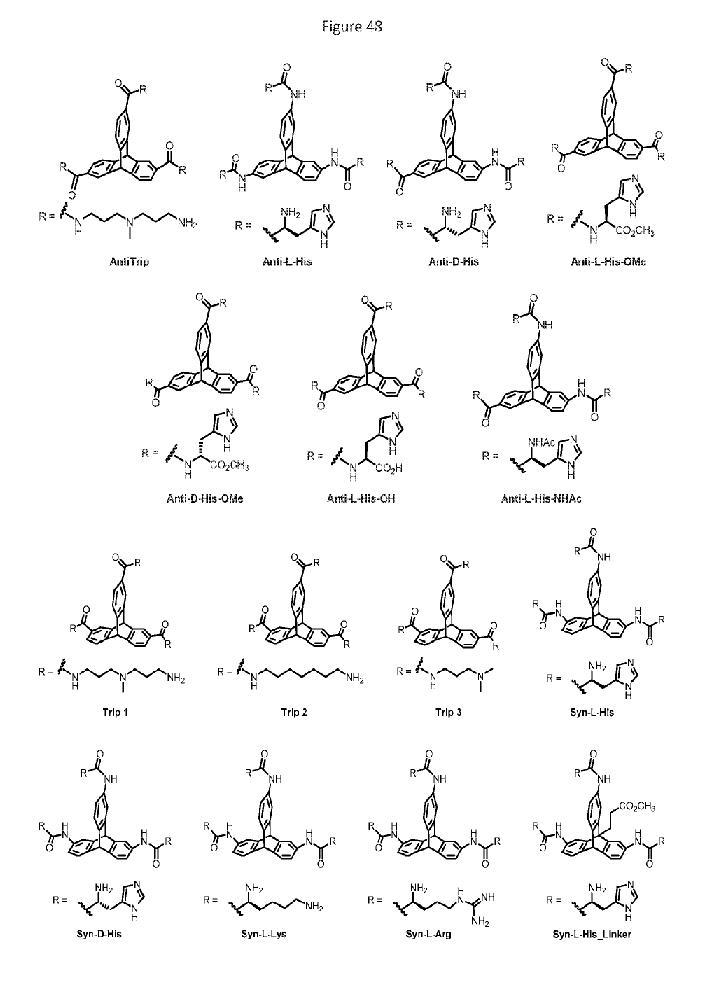

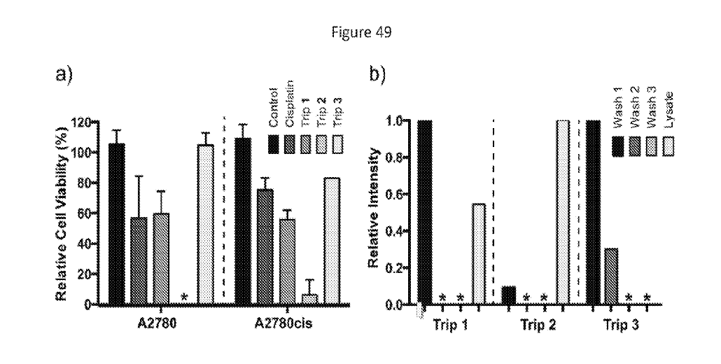

Next, we tested the ability of Trip 3 and 4 to induce fluorescence-quenching upon junction formation using a double labelled (CAG)⋅(CTG) repeat oligonucleotide. The TNR oligonucleotide was labelled with a FAM fluorophore on the 5′-end and an IowaBlack quencher on the 3′-end (TNR*) (FIG. 44a). Formation of the junction results in little to no fluorescence due to the proximity of the quencher and fluorophore, resulting in efficient contact quenching. The junction was incubated with the 10 bp inhibitor strand (I10). As expected, titration of HO resulted in an increase in fluorescence, consistent with disruption of the folded junction, in which the fluorophore and quencher are separated in space (TNR*-I10) (FIG. 44b). Pre-formation of the open inhibited state of the junction (TNR*-I10) followed by titration of Trip 3 or Trip 4, resulted in a concentration dependent decrease in fluorescence (FIG. 44c). The decrease in signal indicates that the fluorophore and quencher are in close proximity due to refolding of the junction. Due to the competitive nature of this assay, only apparent K_{d} values may be calculated. The apparent K_{d} values of Trip 3 and Trip 4 were determined to be 52.8 nM and 2.36 μM, respectively.

Temperature-dependent circular dichroism (CD) was used to further characterize the interaction of Trip 3 and Trip 4 with the TNR junction. The CD spectra are indicative of B-DNA, showing positive signals at 280 nm due to base stacking and negative signals at 250 nm due to the right-handed helicity (FIG. 44d-f). As the temperature increased, the positive at 280 nm decreased and the negative at 250 nm increased, consistent with melting of the DNA structure (FIG. 44d). Upon addition of Trip 3 and Trip 4, distinct spectral changes are observed, resulting in an increased signal at 240 nm as well as a more positive signal at 280 nm and more negative signal at 210 nm (FIG. 44e,f). The increase at 280 nm is consistent with enhanced base stacking and increased helicity. Studies have shown that CAG slip-outs in a 3WJ are less paired and adopt more of an open loop structure. The increased helicity observed in the CD spectrum may be due to increased base pairing interactions in the slip-out region upon addition of Trip 3 or 4.

In summary, we have described triptycene-based molecules that binds to d(CAG)⋅(CTG) trinucleotide repeats. Trip 3 and Trip 4 bind to a model (CAG)⋅(CTG) repeat as determined by gel shift and fluorescence-quenching experiments. The CD spectra are also consistent with enhanced helicity of the slipped out junction upon addition of Trip 3 and 4. This new class of nucleic acid binding small molecule may serve as inspiration for creating valuable probes for diseases associated with trinucleotide repeat expansions. Trinucleotide repeat nucleic acid sequences are associated with a large number (>30) of inherited human muscular and neurological diseases. The trinucleotide repeat tract length is dynamic and often correlates with disease severity, where short stable tracts are commonplace in the non-affected population. Longer unstable triplet repeat tracts are more prone to expansion as opposed to contraction, in addition to being predisposed to generational transmission. Trincleotide repeat repair outcomes are also affected by structural features present in slipped sequences, where the structure may determine which proteins are recruited for repair. Stabilization of a particular structure could lead to increased repair of these slipped-out junction. Addition of ligands that bind to these junctions may affect repair outcomes as well as recruitment of proteins. Small molecule probes will provide valuable tools to study these processes. Small molecules binding and stabilization or modulation of these dynamic structures could lead to the development of therapeutic agents for their associated diseases.

General Methods:

TNR DNA 3WJ (5′-GCGGAGCAGCCCTTGGGCAGCACCTTGGTGCTGCTCCGC-3′) and DNA inhibitor 10 (5′-GCTGCTCCGC-3′) were purchased from Integrated DNA Technologies (IDT). HPLC-purified TNR DNA 3WJ oligo modified with a 5′-FAM and a 3′-IowaBlack was purchased from IDT. Amino acids (Boc-Arg(Mtr)-OH, Boc-Lys(Boc)-OH, and Boc-His-OH were purchased from Merck Millipore Novabiochem (Billerica, Mass., USA). (1-[Bis(dimethylamino)methylene]-1H-1,2,3-triazolo[4,5-b]pyridinium 3-oxid hexafluorophosphate) (HATU) was purchased from GenScript (Piscataway, N.J., USA). All other reagents were purchased from Sigma Aldrich (St. Louis, Mo., USA) and used without further purification. Reactions requiring anhydrous conditions were run under argon with solvents purchased from Fisher dried via an alumina column. Thin-layer chromatography was done using Sorbent Technologies (Norcross, Ga., USA) silica plates (250 μm thickness). Milli-Q (18 MS2) water was used for all solutions (Millipore; Billerica, Mass., USA).

¹H and ¹³C NMR were recorded on a Bruker UNI 500 NMR at 500 and 125 MHz, respectively. Matrix-assisted laser desorption ionization (MALDI) mass spectra were obtained on a Bruker Ultraflex III MALDI-TOF-TOF mass spectrometer (Billerica, Mass., USA) using α-cyano-4-hydroxycinnamic acid (CHCA). Low resolution electrospray ionization (ESI) mass spectra (LRMS) were collected on a Waters Acquity Ultra Performance LC (Milford, Mass., USA). High resolution mass spectra were obtained at the University of Pennsylvania Mass Spectrometry Center on a Waters LC-TOF mass spectrometer (model LCT-XE Premier) using electrospray ionization in positive or negative mode, depending on the analyte. High-performance liquid chromatography was performed on a JASCO HPLC (Easton, Md., USA) equipped with a Phenomenx (Torrance, Calif., USA) column (Luna 5μ C18(2) 100 A; 250×4.60 mm, 5 μm) using aqueous (H₂O+0.1% CF3CO2H) and organic (CH3CN) phases. Circular dichroism experiments were performed on a JASCO J-1500 CD Spectrometer (Easton, Md., USA) using a 0.1 cm path length quartz cuvette. Fluorescence measurements were collected on a Tecan M1000 plate reader (Mannedorf, Switzerland). HPLC chromatograms were obtained at all wavelengths from 200 to 800 nm (bottom plot). 254 and 214 nm were chosen as virtual channels to show the absorbances at those two specific wavelengths (top plot). The blue line corresponds to 254 nm and the red line corresponds to 214 nm (top plot). The lamps used were D2+W with a slit width of 4 nm. A flow rate of 1.00 mL/min was used over 35 minutes. The method began at 10% acetonitrile and 90% water+0.1% TFA. The gradient was increased to 25% acetonitrile over 25 minutes and then increased to 100% acetonitrile.

Synthesis:

General Procedure:

To a solution of 5 (0.08 mmol) in DMF (1 mL) was added HATU (0.256 mmol) and DIEA (0.496 mmol) and was stirred at room temperature for 5 minutes. The corresponding amino acid (0.256 mmol) was added and stirred overnight. The reaction mixture was concentrated under vacuum, then diluted with water and extracted with EtOAc. The crude product was suspended in 4M HCl in dioxane and stirred for 2 hours. The mixture was concentrated, dissolved in acidic water (0.1% TFA) and washed with CH₂Cl₂. The product was purified on a JASCO High-Performance Liquid Chromatography (HPLC) instrument on a C18 column using aqueous (H₂O+0.1% TFA) and organic (CH₃CN) phases.

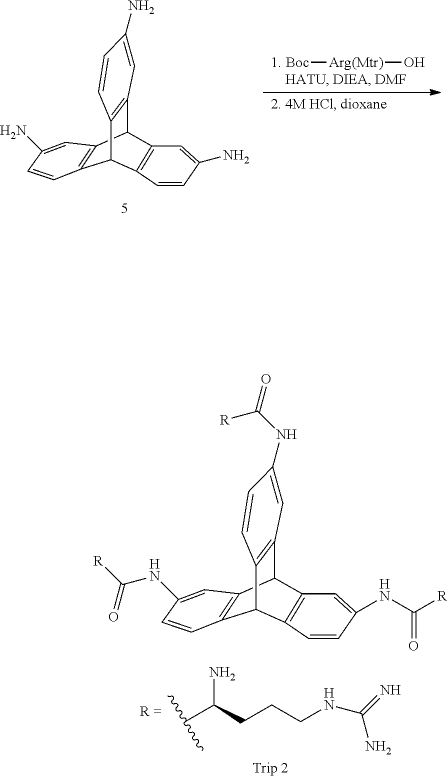

### (2S,2'S,2″S)—N,N′,N″-(9,10-dihydro-9,10-[1,2]benzenoanthracene-2,7,15-triyl)tris(2-amino-5-guanidinopentanamide) (2)

¹H NMR (500 MHz, D₂O) δ 7.65 (s, 3H), 7.51-7.50 (d, 3H) 7.12-7.10 (d, 3H), 5.72 (s, 1H), 5.66 (s, 1H), 4.13-4.10 (t, 3H), 3.23-3.20 (t, 3H), 2.03-1.98 (m, 6H), 1.72-1.66 (m, 6H); HRMS m/z calcd for C₃₈H₅₆H₁₅O₃³⁺ [M+3H]³⁺ 256.8225, observed 256.8231.

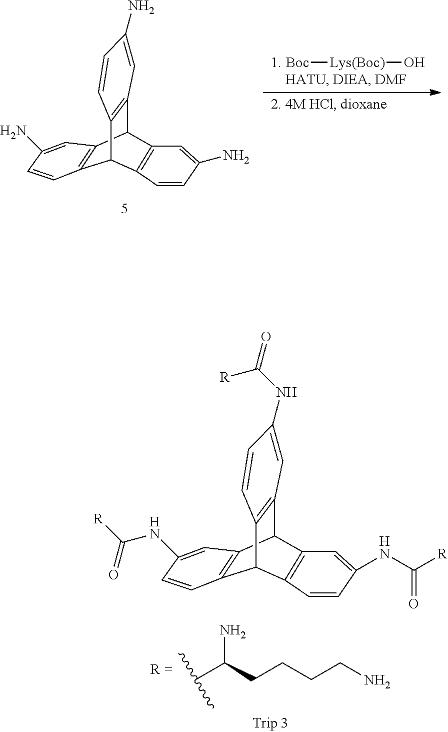

### (2S,2'S,2″S)—N,N′,N″-(9,10-dihydro-9,10-[1,2]benzenoanthracene-2,7,15-triyl)tris(2,6-diaminohexanamide) (3)

¹H NMR (500 MHz, D₂O) δ 7.65 (s, 3H), 7.52-7.50 (d, 3H) 7.13-7.11 (d, 3H), 5.73 (s, 1H), 5.67 (s, 1H), 4.11-4.09 (t, 3H), 2.99-2.96 (t, 6H), 2.01-1.96 (m, 6H), 1.17-1.67 (m, 6H), 1.51 (m, 6H); HRMS m/z calcd for C₃₈H₅₄N₉O₃⁺ [M+H]⁺ 684.4344, observed 684.4357.

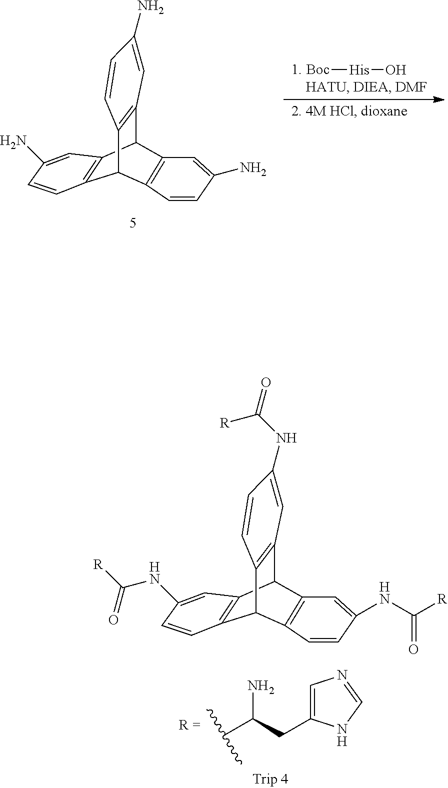

### (2S,2′S,2″S)—N,N′,N″-(9,10-dihydro-9,10-[1,2]benzenoanthracene-2,7,15-triyl)tris(2-amino-3-(1H-imidazol-5-yl)propanamide) (4)

¹H NMR (500 MHz, D₂O) δ 8.58 (s, 3H), 7.65 (s, 3H) 7.54-7.52 (d, 3H), 7.40 (s, 3H), 7.07-7.05 (d, 3H), 5.75 (s, 1H), 5.69 (s, 1H), 4.40-4.36 (t, 3H), 3.47-3.45 (d, 6H); ¹³C NMR (500 MHz, D₂O) δ 166.63, 145.73, 142.70, 134.65, 133.19, 126.72, 124.29, 118.65, 118.15, 117.52, 115.20, 52.93, 51.60, 26.58; MALDI-TOF m/z calcd for C₃₈H₃₈H₁₂O₃ [M+H] 710.80, observed 711.397, HRMS m/z calcd for C₃₈H₃₉H₁₂O₃⁺ [M+H]⁺ 711.3263, observed 711.3274.

Gel Shift Assay:

All gel shift experiments were conducted in 50 mM sodium phosphate buffer, pH 7.2. The screening gel was run by incubating TNR 3WJ (0.5 μM) with inhibitor strand 10 bases long (1.5 μM) in a 20 μL solution at room temperature for 2 hours. Triptycenes were then added at a final concentration of 10 μM and incubated for 2 hours. Samples were run on a 20% non-denaturing polyacrylamide gel (19:1 monomer:bis) at 50V in 1×TBE buffer at 4° C. for 10 hours. Gels were stained with SYBR Gold for 10 minutes and visualized using a BioRad GelDoc XR+ imager.

Inhibitor strand titration gel was run by incubating TNR 3WJ (0.5 μM) with increasing concentrations of inhibitor strand in a 20 μL solution at room temperature for 2 hours. Samples for compound titration were prepared by incubating TNR 3WJ (0.5 μM) with inhibitor strand (1.5 μM) for 2 hours followed by titration of Trip 4 and incubation at room temperature for 2 hours. Samples were run on a gel as described above.

Fluorescence Quenching Experiments:

All binding experiments were conducted in 50 mM sodium phosphate buffer, pH 7.2. Fluorescence measurements were recorded with excitation at 495 nm and emission at 520 nm using 5 nm bandwidths. Inhibitor strand binding curves were obtained by adding 1 μL of increasing concentrations of inhibitor strand to 19 μL of 120 nM TNR 3WJ. Samples were incubated for 2 hours and ran in triplicate in a 384-well plate. Inhibitor strand displacement curves were obtained by incubating 120 nM aptamer with 1 μM inhibitor 10 for 2 hours, followed by addition of increasing concentrations of 4. Samples were incubated for 2 hours and measured in triplicate.

Circular Dichroism:

DNA was suspended at 6 μM in 50 mM sodium phosphate buffer, pH 7.2 and annealed by heating to 90° C. for 5 min, cooled to room temperature slowly, then to 4° C. Spectra were measured every 0.5 nm between 350 nm and 200 nm with an 8 s averaging time. Samples were incubated at each temperature for 20 minutes prior to scan. Samples were incubated with ligand (24 μM) at room temperature for 1 hour. All CD spectra were buffer corrected and converted to molar ellipticity.

### Example 4

### Synthesis of 9-Substituted Triptycene Building Blocks for Solid-Phase Diversification and Nucleic Acid Junction Targeting

Figures, tables and references are as published in Yoon et al., Organic Letters, 2016(18):1096, published on Feb. 17, 2016.

Triptycenes have been shown to bind nucleic acid three-way junctions, but rapid and efficient methods to diversify the triptycene core are lacking. An efficient synthesis of a 9-substituted triptycene scaffold is reported that can be used as a building block for solid-phase peptide synthesis and rapid diversification. The triptycene building block was diversified to produce a new class of tripeptide-triptycenes, and their binding abilities toward d(CAG)⋅(CTG) repeat junctions were investigated.

Nucleic acid junctions play important roles in many biological events. Three-way junctions (3WJs) have diverse architectures and are found in DNA and RNA, where they often serve as important structural elements. Several small molecules are known to bind to nucleic acid junctions. However, these molecules often lack specificity, leading to binding of various structures.

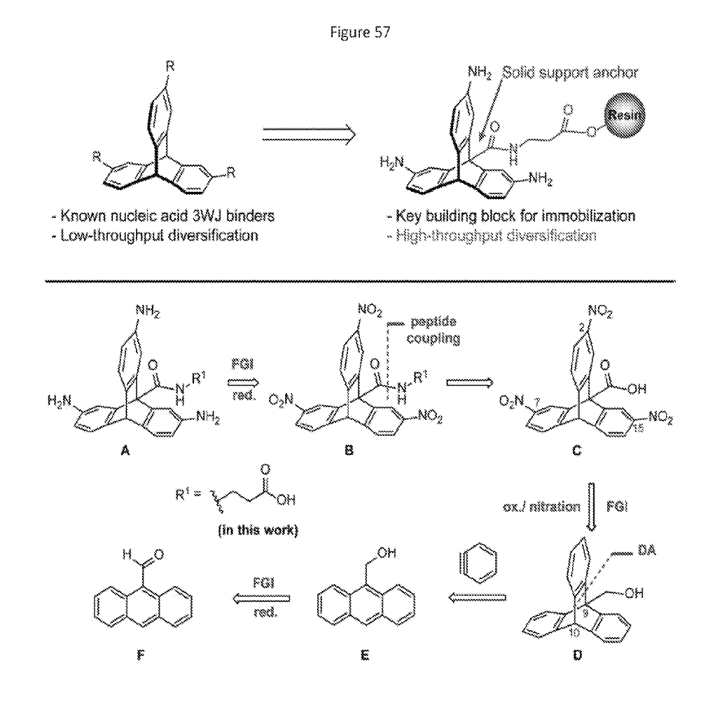

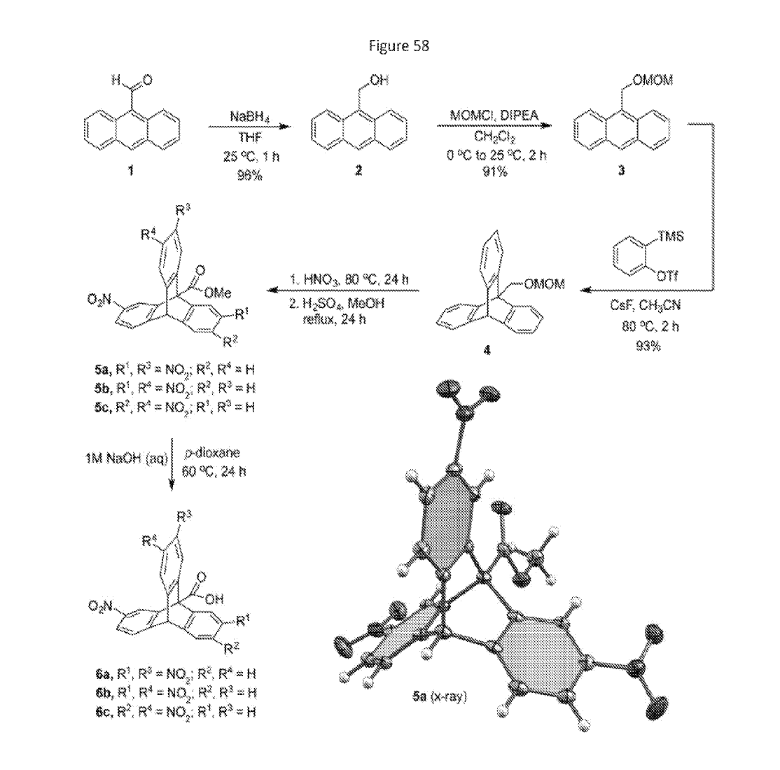

Efficient strategies for triptycene diversification are needed to accelerate the discovery of new nucleic acid junction binders with enhanced specificity and binding properties. Triptycene building blocks that are amenable to immobilization on a solid support would allow for rapid diversification and compound library construction (FIG. 57). To immobilize triptycene, we designed and synthesized a 9-substituted derivative that provides a point of attachment at the bridgehead, maintaining the C3 symmetry. Although triptycene has been extensively modified for use in materials chemistry applications, functionalization at the C9-position of triptycene has rarely been reported.

A carboxylic acid was chosen for functionalization at the C9 tertiary carbon of triptycene due to its versatility of conversion into other functional groups, such as aldehyde, haloalkane, ester, and amide. The carboxylic acid group may also be removed via decarboxylation at a later stage. More importantly, the carboxylic acid group has been extensively employed for directed C-H bond functionalization reactions, which could prove valuable during future triptycene diversification efforts.

Our synthetic plan (FIG. 57) relies on reduction of the nitro groups on precursor B to yield building block A. Further disconnection of the amide bond at the bridgehead position affords carboxylic acid C. A β-alanine ethyl ester was coupled to the carboxylic acid on C. O-Directed nitration was envisioned to regioselectively build three nitro groups onto triptycene D. In addition to nitration, simultaneous oxidation of the alcohol of D to the desired bridgehead carboxylic acid was anticipated. Next, disconnection at C9 and C10 affords benzyne and anthracene E precursors, which could be assembled via a Diels-Alder reaction. Precursor E was prepared by reduction of aldehyde F. Early stage functionalization of triptycene at C9 would provide an efficient route to a triptycene building block suitable for solid-phase immobilization and further diversification.

Commercially available anthracene-9-carbaldehyde 1 was employed as a starting material. Reduction of 1 using sodium borohydride afforded anthracen-9-ylmethanol 2 in 96% yield within 1 h (FIG. 58). Prior to the addition of the Kobayashi benzyne precursor, the primary alcohol was protected with a MOM group to prevent electrophilic attack by benzyne. The Diels-Alder reaction between 3 and benzyne, which was generated in situ from 2-(trimethylsilyl)phenyltrifluoromethanesulfonate and cesium fluoride, led to the efficient formation of triptycene 4 in high yield.

Treatment of 4 with nitric acid resulted in nitration of the aromatic rings. During the nitration reaction, the protecting group on the alcohol was simultaneously deprotected and oxidized to the carboxylic acid, providing 6a along with two other isomers 6b and 6c. The nitrated triptycene isomers proved inseparable by silica gel chromatography. Acid-catalyzed esterification of the crude mixture provided ester isomers 5a-c, which were separated via silica gel chromatography. The structures of ester isomers 5a-c were confirmed by twodimensional NMR spectroscopy, HMBC, and HSQC. Single crystals of 5a were grown in CHCl3/CH2Cl2/CH3OH, and the structure was determined by X-ray crystallography (FIG. 58). Following separation of each isomer, saponification was performed to convert the ester to a carboxylic acid for coupling to an amine linker. Nitration on the α-carbon was not observed due to its higher electronegativity compared to that of β-carbons.

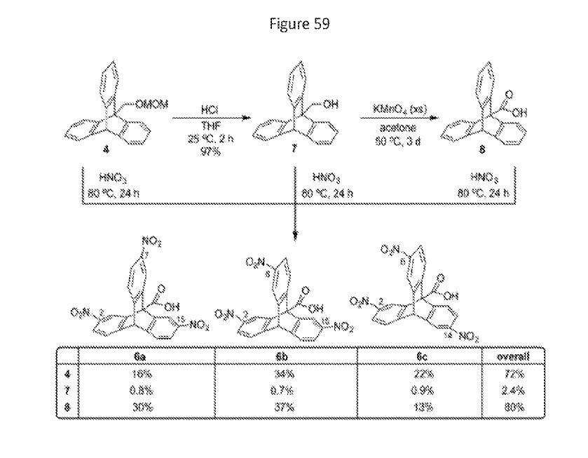

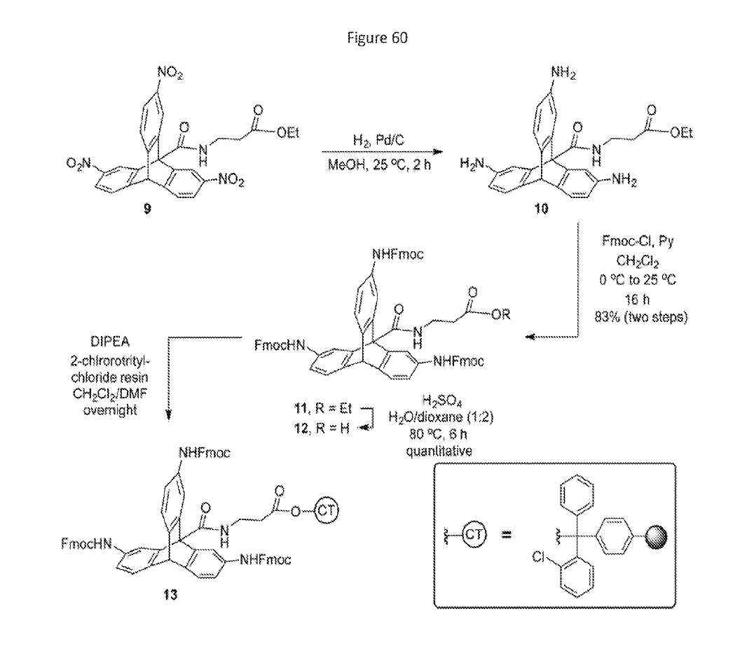

To investigate the O-directing effect observed during nitration and to further reduce the number of undesired side products, compound 8, containing a carboxylic acid at the C9 position, was prepared by deprotection of 4 followed by KMnO4 oxidation (FIG. 59). Compounds 4, 7, and 8 were treated with excess nitric acid at 80° C. for 24 h, and the crude mixtures were analyzed by HPLC using 9,10-diphenylanthrancene as an internal standard. HPLC analysis demonstrated that nitration of 8 led to fewer side products compared to nitration of 4 and 7.

Interestingly, nitration of 7 produced little of desired products 6a-c. The composition of 6a and 6c significantly changed compared to that from the nitration of 4 and 8, and the overall yield increased for the nitration of 8. Attempts were made to increase the proportion of 6a over that of the other isomers. The highest ratio of 6a to 6b achieved using this nitration method was 0.33. The introduction of a carboxylic acid at the C9 position of triptycene significantly increased the ratio of 6a to 6b to 0.81. These observations are consistent with the carboxylic acid functioning as a directing group during the nitration reaction.

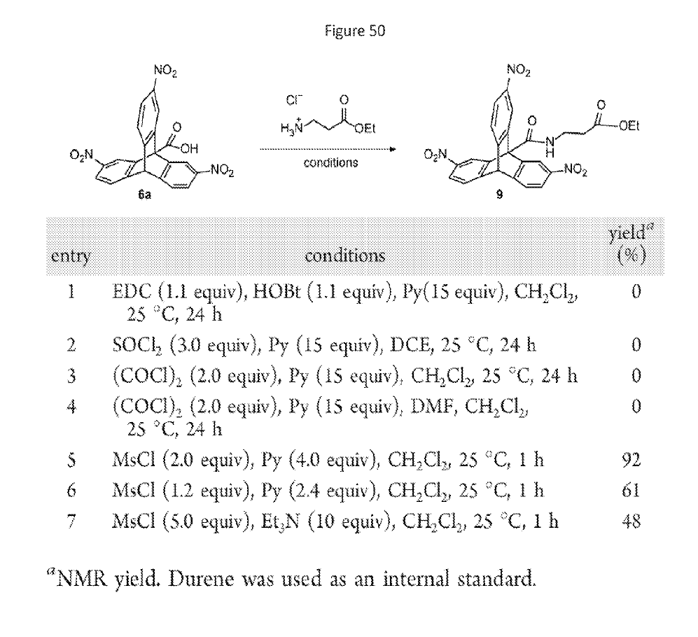

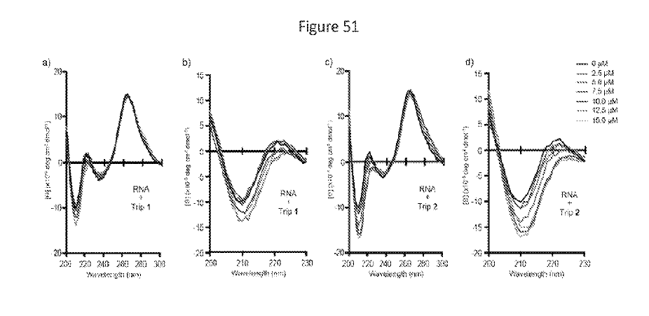

Isomer 6a was chosen for further elaboration due to its 3-fold symmetry, which is complementary to that of nucleic acid 3WJs. To extend the length of the linker at the 9-position, several standard reaction conditions for amide bond formation were examined. However, the amidation reaction proved recalcitrant, and all attempted conditions resulted in unreacted starting material (FIG. 50, entries 2-4). The coupling of 9-triptycenecarboxylic acid derivatives with EDC has been previously reported. However, this method was not reproducible using 6a as the starting material (FIG. 50, entry 1). Our results suggested that the sterically hindered environment around the carboxylic acid prevents coupling of amines under standard conditions, possibly due in part to the bulky active ester intermediates. After a comprehensive literature search, we were inspired by Nicolaou's use of methanesulfonyl chloride (MsCl) in the total synthesis of the CP molecules to overcome limitations of a difficult Arndt-Eistert homologation on sterically encumbered carboxylic acids. Triethylamine and MsCl were added to 6a followed by addition of β-alanine ethyl ester hydrochloride, which was pretreated with triethylamine at 0° C. After being warmed to room temperature, 9 was synthesized in 48% yield (FIG. 50, entry 7). However, complete conversion of the starting material was not achieved under these conditions. To drive the reaction to completion, the base was changed to pyridine, which is less sterically hindered and allows for access to the carboxylic acid near the bridgehead position. The solvent was also changed to dichloromethane due to solubility issues. These changes led to completion of the reaction within 1 h after warming to room temperature and a substantial increase in the yield to 92% (FIG. 50, entry 5). A decrease in the equivalence of MsCl and pyridine decreased the yield to 61% (FIG. 50, entry 6). Among the various amide bond-forming reaction conditions tested on triptycene 6a, only the mesylation route afforded the desired product in high yield.

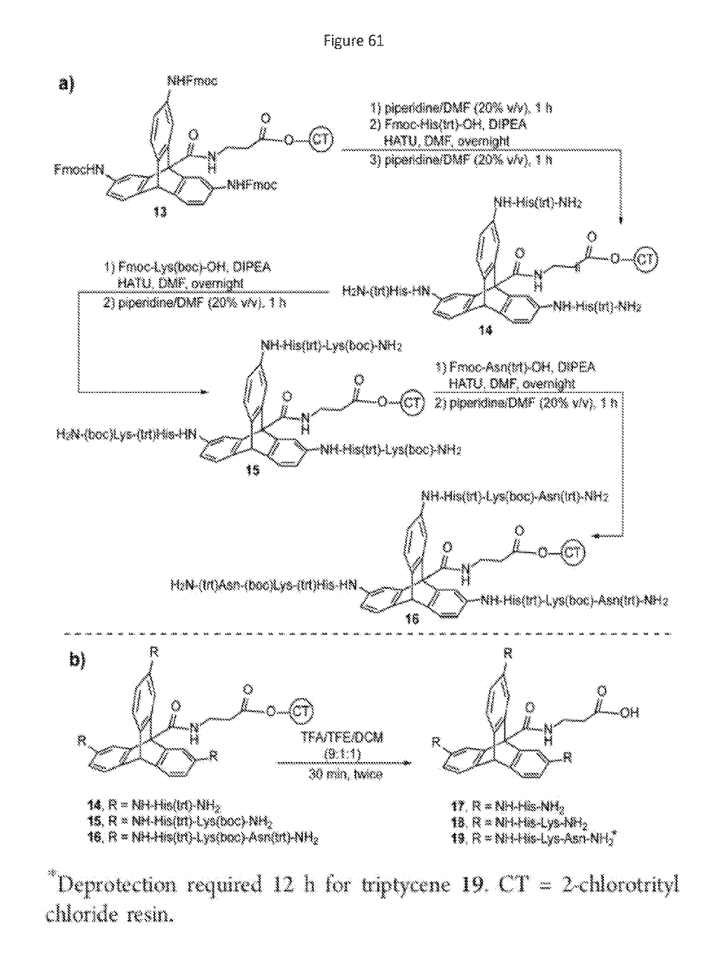

Pd/C-catalyzed hydrogenation of 9 led to reduction of the three nitro groups to afford triaminotriptycene 10. Next, the free amines were protected with Fmoc groups by treatment with Fmoc chloride and pyridine. The linker ester group was hydrolyzed in the presence of sulfuric acid and water to produce acid 12. The free carboxylate of fully protected building block 12 allowed for attachment to 2-chlorotrityl chloride resin, which is compatible with Fmoc deprotection chemistry (FIG. 60). After attachment to resin, the Fmoc groups were deprotected using 20% piperidine in dimethylformamide to generate the free amines. The corresponding Fmocprotected amino acid was preactivated with HATU and N,Ndiisopropylethylamine (DIPEA) and added to the deprotected triptycene on resin. L-Histidine, L-lysine, and L-asparagine were selected for attachment to the triptycene arms. The deprotection and coupling steps were repeated until the desired sequence of amino acids was achieved (FIG. 61a). Once the desired peptide was synthesized on solid phase, the triptycene derivatives were cleaved from the resin with simultaneous deprotection of the amino acid side chain protecting groups by treatment with a cleavage solution (9:1:1 trifluoroacetic acid (TFA)/2,2,2-trifluoroethanol (TFE)/dichloromethane). Asparagine, which was coupled at the N-terminus, required longer cleavage times due to the slow deprotection rate of the trityl group close to the amino group (FIG. 61b). Each compound was purified by preparative reversed-phase HPLC and analyzed by analytical HPLC and MALDI-MS.

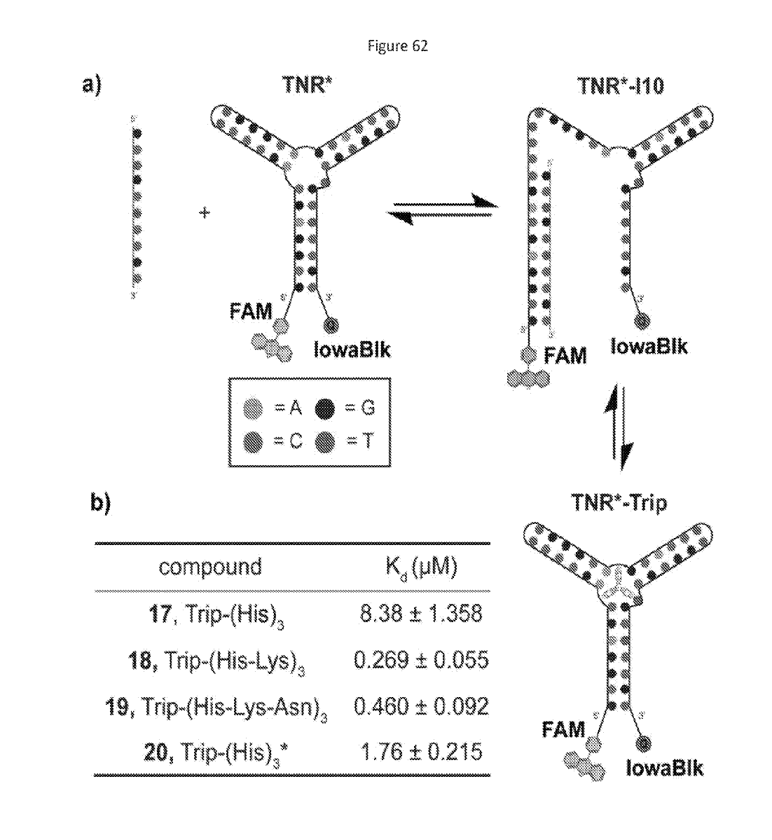

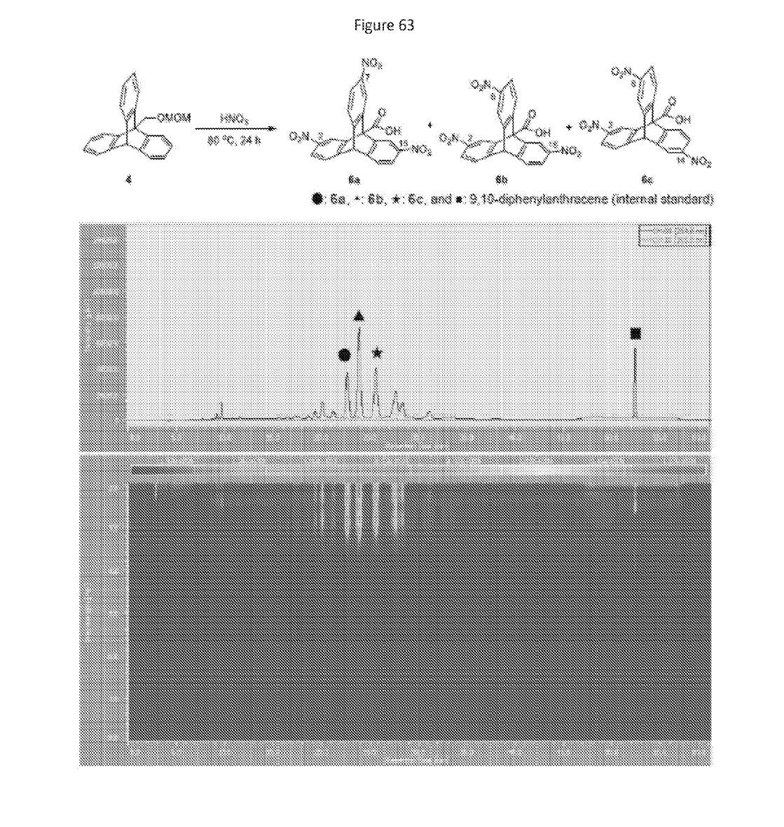

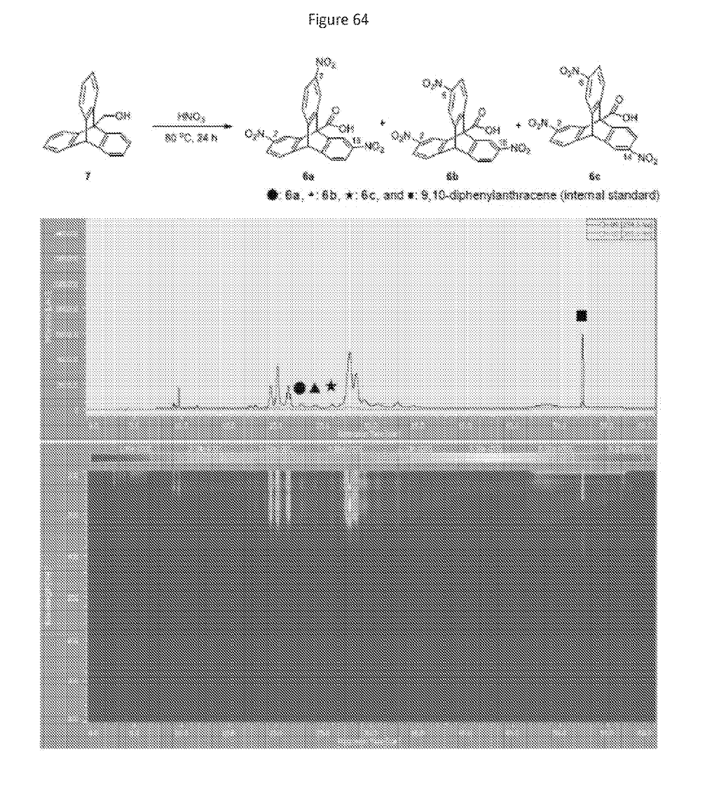

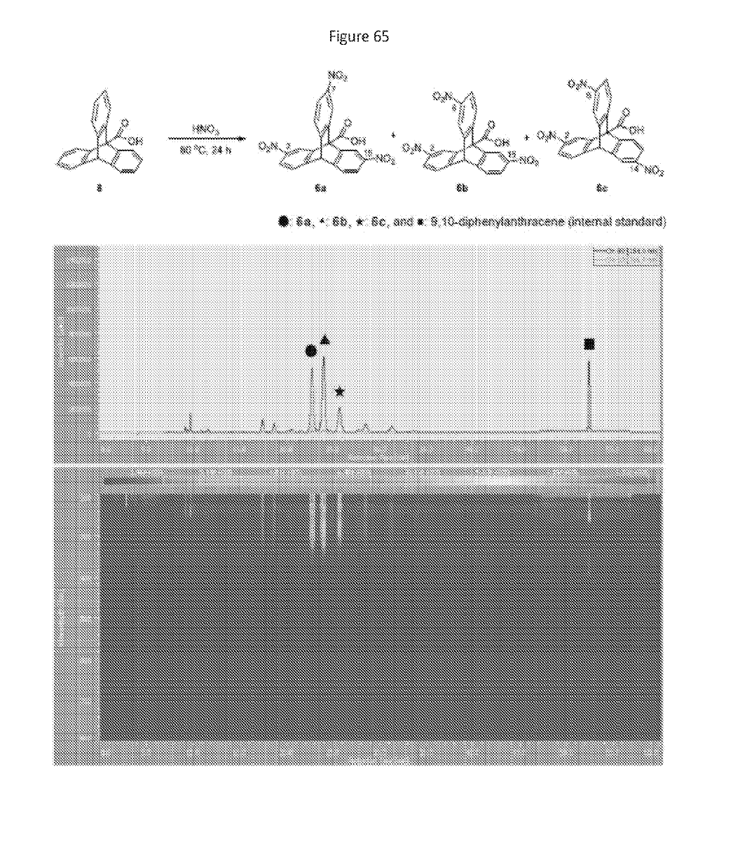

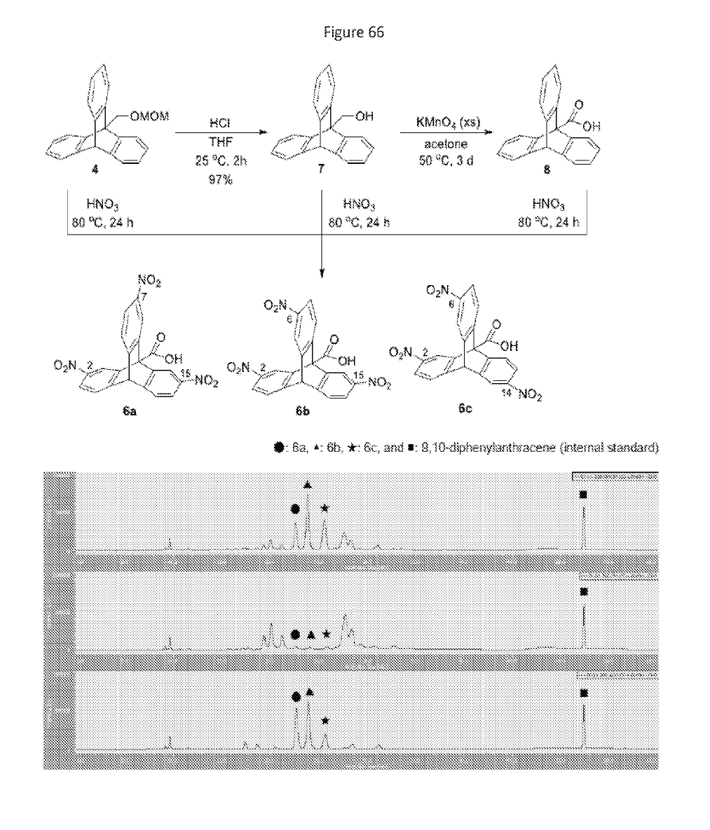

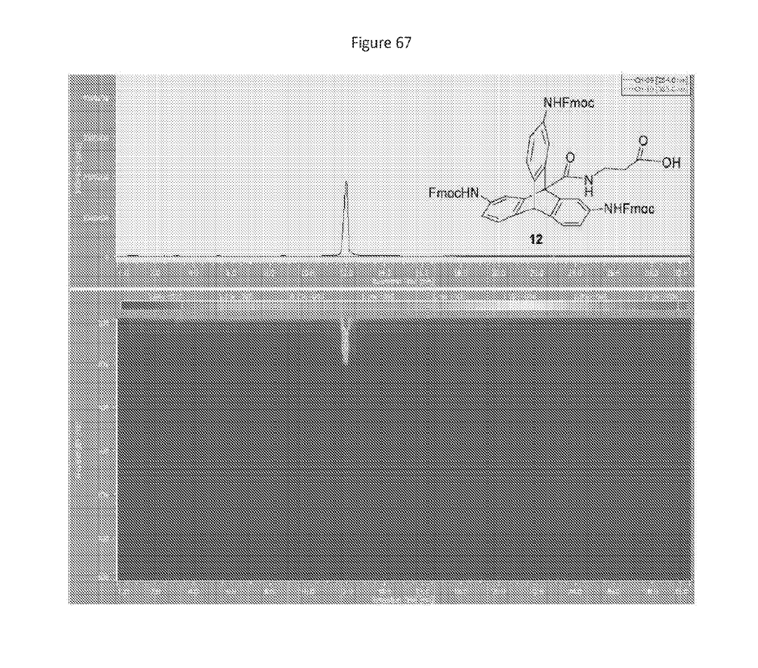

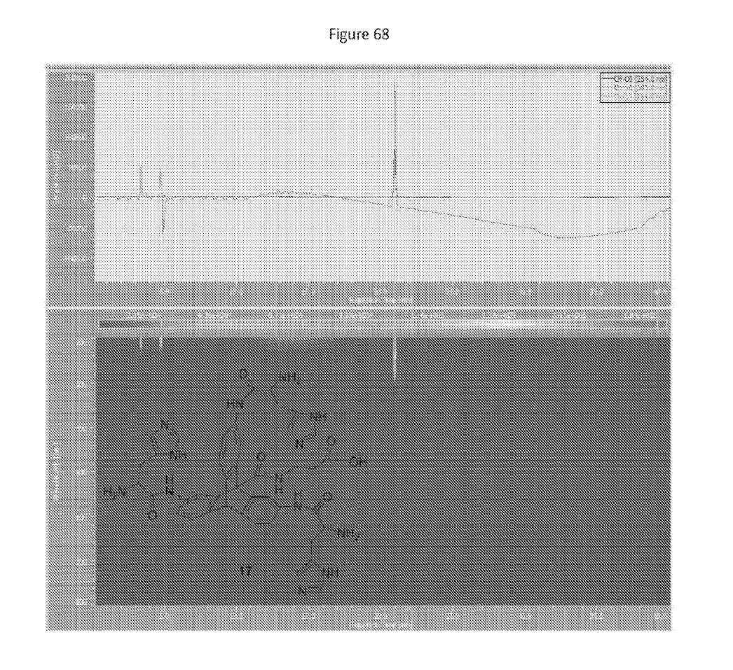

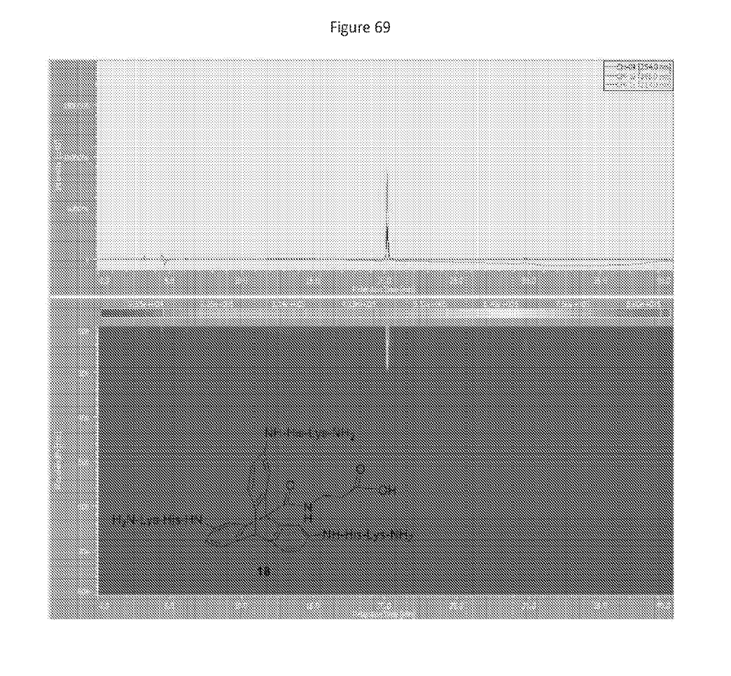

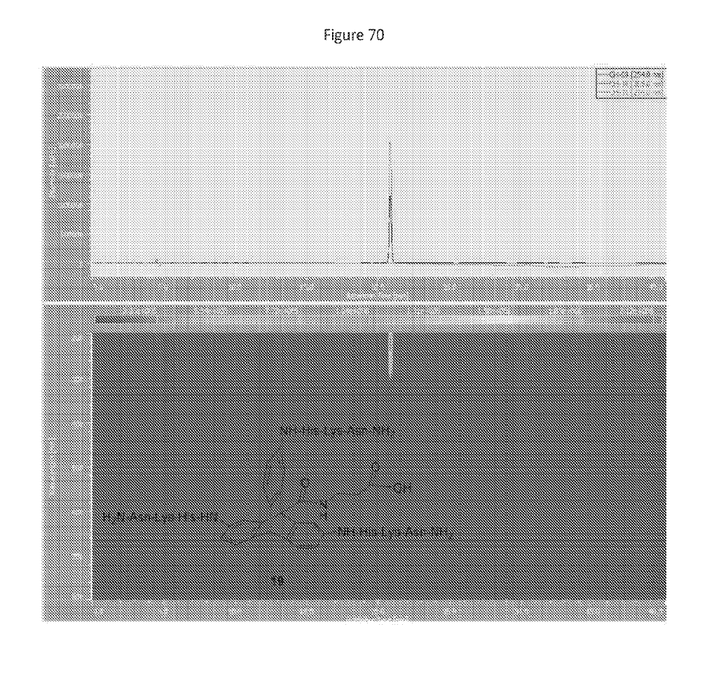

Triptycenes 17-19 were evaluated for binding toward a d(CAG)⋅(CTG) trinucleotide repeat junction using a previously developed fluorescence-quenching experiment. The binding of triptycenes 17-19 were compared to a previously reported triptycene that binds to the junction. The previously reported junction binder (20) is analogous to 17 but lacks the linker at the 9-position. A d(CAG)⋅(CTG) repeat junction was labeled with a fluorophore (FAM) and a quencher (IowaBlk). This labeled 3WJ was preincubated with a 10 bp inhibitor (I10) strand that is complementary to the junction. Hybridization of the inhibitor strand to the junction results in an open form, leading to an increase in fluorescence (FIG. 62a). Triptycenes 17-20 were added to the preincubated fluorescent form. Binding of the triptycenes leads to displacement of I10 and reformation of the 3WJ, resulting in a decrease in fluorescence. The K value for triptycene 17 was determined to be 8.38 μM and exhibited a slight decrease in binding compared to that of triptycene 20 (i.e., K_{d} value of 1.76 μM). Triptycenes 18 and 19, containing di- or tripeptides substituents, exhibited enhanced binding affinity toward the junction compared to that of 20 with K_{d} values of 0.27 and 0.46 μM, respectively (FIG. 62b). The presence of lysine appears to play an important role in binding to the junction and will be investigated in future studies.

In summary, we have developed a synthetic approach for preparing new 9-substituted triptycene building blocks. This approach enables solid-phase diversification of triptycene. During the synthesis, O-directed nitration was observed from the MOM protected primary alcohol (4), primary alcohol (7), and carboxylic acid (8) at the C9 position of triptycene. These results indicated that the carboxylic group increased the ratio of nitration on β-carbons toward the linker position, pointing to a possible carboxylic acid directing effect. In addition, a key amide bond formation was achieved on a sterically hindered and geometrically fixed tertiary carboxylic acid using a unique MsCl activation strategy. This may be regarded as a general strategy toward functionalization of extremely sterically encumbered tertiary carboxylic acids. For diversification of the new triptycene building block, three amino acids were utilized including histidine, lysine, and asparagine to produce trisubstituted triptycenes 17-19. The binding ability of the synthesized triptycene derivatives toward a d(CAG)⋅(CTG) trinucleotide repeat junction was evaluated, and triptycenes 18 and 19 exhibited better binding affinity to the junction compared to that of a previously reported triptycene with no linker (20). This new synthetic strategy provides rapid and efficient access to triptycene building blocks, enabling high throughput diversification for rapid evaluation of potential junction binders and other medicinal chemistry targets.

General Methods:

All commercial reagents and solvents were used as received. 9-anthracenecarboxaldehyde, sodium borohydride, N,N-diisopropylethylamine, chloromethyl methyl ether, beta-Alanine ethyl ester hydrochloride, Fmoc chloride, palladium on activated carbon, cesium fluoride, 2-(trimethylsilyl)phenyl trifluoromethanesulfonate, and nitric acid from Aldrich, 1-[bis(dimethylamino)methylene]-1H-1,2,3-triazolo[4,5-b]pyridinium3-oxide hexafluorophosphate (HATU) from Oakwood Products, Inc., 2-chlorotrityl chloride resin from Advanced ChemTech, chloroform-d, methylene chloride-d2, dimethylsulfoxide-d6, and acetone-d6 from Cambridge Isotope Laboratories Inc. were purchased. HPLC-purified TNR DNA 3WJ oligo modified with a 5′-FAM and a 3′-IowaBlack (5′-(FAM)-GCGGAGCAGCCCTTGGGCAGCACCTTGGTGCTGCTCCGC-(IowaBlk)-3′) and DNA inhibitor 10 (5′-GCTGCTCCGC-3′) were purchased from Integrated DNA Technologies (IDT).

Flash column chromatography was performed using Silicycle silica gel (55-65 Å pore diameter). Thinlayer chromatography was performed on Sorbent Technologies silica plates (250 μm thickness). Proton nuclear magnetic resonance spectra (1H NMR) and Carbon nuclear magnetic resonance (13C NMR) spectra were recorded on a Bruker DMX 500. High-resolution mass spectrometry analysis was obtained by Dr. Rakesh Kohli at the University of Pennsylvania's Mass Spectrometry Service Center on a Waters LC-TOF mass spectrometer (model LCT-XE Premier) using electrospray ionization. High-performance liquid chromatography (HPLC) chromatograms were recorded and triptycenes 17-19 was purified on JASCO HPLC (Easton, Md.) equipped with a Phenomenx (Torrance, Calif.) column (Analytical: Luna 5μ C18(2) 100 A; 250×4.60 mm, 5 μm Semi-prep: 5μ C18(2) 100 A; 250×10.00 mm, 5 μm) using aqueous (H2O+0.1% CF3CO2H) and organic (CH3CN) phases. Matrix-assisted laser desorption ionization (MALDI) mass spectra were recorded on a Bruker Ultraflex III MALDITOF-TOF mass spectrometer (Billerica, Mass.) using α-cyano-4-hydroxycinnamic acid (CHCA). Fluorescence measurements were obtained on a Tecan M1000 plate reader (Mannedorf, Switzerland).

### Experimental Procedures

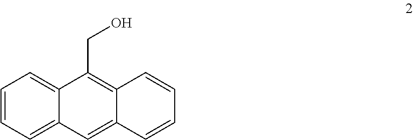 [SMILES: OCc1c2ccccc2cc2ccccc12]

### anthracen-9-ylmethanol (2)

To 4.9 g (23.76 mmol) of anthracene-9-carbaldehyde (1) in THF (50 mL) was added 1.35 g (35.64 mmol) of NaBH4. The mixture was stirred for 1 h at 25° C. The mixture was poured into water (400 mL) resulting in a yellow precipitate. The yellow solid was filtered off, washed thoroughly with water, and dried. (4.7 g, 96% isolated yield).¹

¹H NMR (500 MHz, CDCl3) δ 8.46 (s, 1H), 8.40 (d, 2H, J=8.8 Hz), 8.02 (d, 2H, J=8.4 Hz), 7.59-7.53 (m, 2H), 7.52-7.46 (m, 2H), 5.65 (s, 2H).

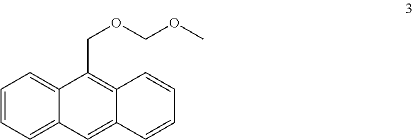 [SMILES: COCOCc1c2ccccc2cc2ccccc12]

### 9-((methoxymethoxy)methyl)anthracene (3)

To 354 mg (1.7 mmol) of anthracen-9-ylmethanol 2 in CH₂Cl₂ was added 1.76 mL (10.2 mmol) of N,N-diisopropylethylamine at 0° C. After stirring for 30 min, 0.4 mL (5.1 mmol) of chloromethylmethyl ether was added to this solution at 0° C. The mixture was stirred for 10 min, warmed to 25° C., and stirred for 18 h. Saturated NH₄Cl (aq) solution was added to the reaction. The organic layer was extracted from the solution, dried with anhydrous sodium sulfate, concentrated in vacuo, and purified by column chromatography using ethyl acetate/hexanes (4%) as the eluent to give 391 mg of 3 (391 mg, 91% isolated yield). Physical Property: Pale yellow solid, m.p.=80-81° C.

TLC: Rf=0.52 (silica gel, 25% ethyl acetate/hexanes).

¹H NMR (500 MHz, CDCl₃) δ 8.53 (d, 2H, J=8.8 Hz), 8.47 (s, 1H), 8.04 (d, 2H, J=8.4 Hz), 7.68-7.62 (m, 2H), 7.58-7.51 (m, 2H), 5.67 (s, 2H), 4.90 (s, 2H), 3.61 (s, 3H). ¹³C NMR (125 MHz, CDCl₃) δ 131.6, 131.3, 129.2, 128.7, 128.4, 126.4, 125.1, 124.4, 95.7, 61.1, 55.8.

IR (neat): 1733, 1446, 1265, 1147, 1093, 1061, 1029, 934, 914, 891, 731, 703, 640 cm⁻¹.

HRMS (ESI) calculated for C₁₇H₁₆NaO₂⁺ [M+Na]⁺ 275.1043, found 275.1055.

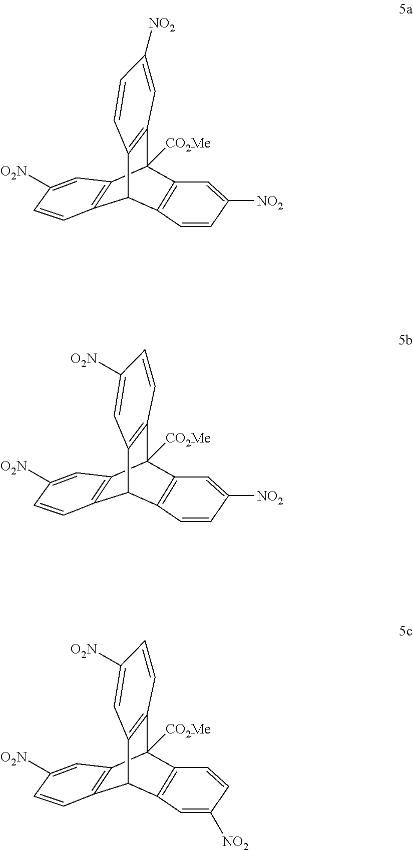 [SMILES: COC(=O)C12c3cc([N+](=O)[O-])ccc3C(c3ccc([N+](=O)[O-])cc31)c1ccc([N+](=O)[O-])cc12 ; COC(=O)C12c3ccc([N+](=O)[O-])cc3C(c3ccc([N+](=O)[O-])cc31)c1ccc([N+](=O)[O-])cc12 ; COC(=O)C12c3ccc([N+](=O)[O-])cc3C(c3cc([N+](=O)[O-])ccc31)c1ccc([N+](=O)[O-])cc12]

### methyl trinitro-9,10-[1,2]benzenoanthracene-9(10H)-carboxylate (5a-5c)

To a round bottom flask was added 3.97 g (12.1 mmol) of 4 and 50 mL of concentrated nitric acid at 25° C. The solution was heated to 80° C. and stirred for 24 h. After the reaction was complete, water was added to the solution. The solution was neutralized with K₂CO₃ and re-acidified with 1M HCl. Ethyl acetate was added to the solution and the organic layer was extracted from the solution. The combined organic solution was dried with anhydrous sodium sulfate, and concentrated in vacuo. To the crude mixture was added 40 mg of H₂SO₄ and 100 mL of anhydrous methanol. The solution was stirred under reflux for 24 h. After the reaction was completed, the solution was cooled, extracted with ethyl acetate, dried with anhydrous sodium sulfate, and then concentrated in vacuo. The crude mixture of 5a-5c was then purified using column chromatography. The composition of each isomer was determined by HPLC analysis.

### methyl 2,7,15-trinitro-9,10-[1,2]benzenoanthracene-9(10H)-carboxylate (5a)

Physical Property: White solid, m.p.=282-283° C.

TLC: R_{f}=0.41 (silica gel, 50% ethyl acetate/hexanes).

¹H NMR (500 MHz, CDCl₃) δ 8.64 (d, 3H, J=2.1 Hz), 8.08 (dd, 3H, J=8.2, 2.2 Hz), 7.64 (d, 3H, J=8.2 Hz), 5.77 (s, 1H), 4.43 (s, 3H).

¹³C NMR (125 MHz, CDCl₃) δ 167.9, 148.9, 146.3, 143.1, 124.9, 122.9, 120.1, 61.4, 53.8, 53.6.

IR (neat): 2924, 1746, 1522, 1455, 1340, 1301, 1274, 1250, 1214, 1166, 1025, 903 cm⁻¹.

HRMS (ESI) calculated for C₂₂H₁₄N₃O₈⁺ [M+H]⁺, no peak matched the calculated exact mass. Hydrolysis of the ester to carboxylic acid 6a was required to obtain the HRMS. See next page for HRMS data on acid 6a.

### methyl (9s,10r)-2,6,15-trinitro-9,10-[1,2]benzenoanthracene-9(10H)-carboxylate (5b)

Physical Property: White Solid, m.p.=163-164° C.

TLC: R_{f}=0.73 (silica gel, 50% ethyl acetate/hexanes).

¹H NMR (500 MHz, CDCl3) δ 8.63 (d, 2H, J=2.0 Hz), 8.33 (d, 1H, J=2.0 Hz), 8.04 (dd, 2H, J=8.1, 2.0 Hz), 7.99 (dd, 1H, J=8.5, 2.0 Hz), 7.89 (d, 1H, J=8.5 Hz), 7.70 (d, 2H, J=8.1 Hz), 5.89 (s, 1H), 4.40 (s, 3H).

¹³C NMR (125 MHz, CDCl3) δ 168.1, 149.6, 147.9, 146.2, 144.4, 142.7, 125.4, 125.1, 122.9, 122.1, 120.1, 119.4, 61.6, 53.5, 53.4.

IR (neat): 2924, 1743, 1519, 1455, 1341, 1297, 1214, 1165, 1071, 1024, 892 cm⁻¹.

HRMS (ESI) calculated for C₂₂H₁₄N₃O₈⁺ [M+H]⁺, no peak matched the calculated exact mass. Hydrolysis of the ester to carboxylic acid 6b was required to obtain the HRMS. See next page for HRMS data on acid 6b.

### methyl (9r,10s)-2,6,14-trinitro-9,10-[1,2]benzenoanthracene-9(10H)-carboxylate (5c)

Physical Property: White Solid, m.p.=161-162° C.

TLC: R_{f}=0.81 (silica gel, 50% ethyl acetate/hexanes).

¹H NMR (500 MHz, CDCl₃) δ 8.65 (d, 1H, J=2.2 Hz), 8.33 (d, 2H, J=2.3 Hz), 8.09 (dd, 1H, J=8.2, 2.2 Hz), 8.05 (dd, 2H, J=8.6, 2.3 Hz), 7.93 (d, 2H, J=8.6 Hz), 7.67 (d, 1H, J=8.2 Hz), 5.80 (s, 1H), 4.37 (s, 3H). ¹³C NMR (125 MHz, CDCl₃) δ 168.1, 149.8, 147.4, 146.24, 146.21, 144.7, 142.3, 125.5, 124.9, 122.9, 122.1, 120.3, 119.3, 61.9, 53.4, 53.3.

IR (neat): 2924, 1744, 1598, 1458, 1438, 1342, 1254, 1165, 1023 cm⁻¹.

HRMS (ESI) calculated for C₂₂H₁₄N₃O₈⁺ [M+H]⁺, no peak matched the calculated exact mass. Hydrolysis of the ester to carboxylic acid 6c was required to obtain the HRMS. See next page for HRMS data on acid 6c.

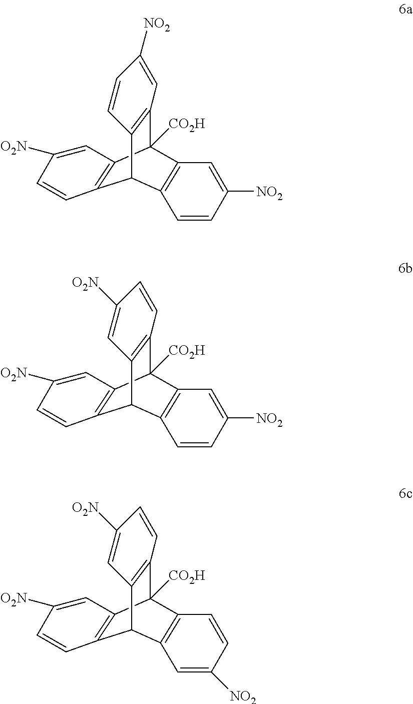 [SMILES: O=C(O)C12c3cc([N+](=O)[O-])ccc3C(c3ccc([N+](=O)[O-])cc31)c1ccc([N+](=O)[O-])cc12 ; O=C(O)C12c3ccc([N+](=O)[O-])cc3C(c3ccc([N+](=O)[O-])cc31)c1ccc([N+](=O)[O-])cc12 ; O=C(O)C12c3ccc([N+](=O)[O-])cc3C(c3cc([N+](=O)[O-])ccc31)c1ccc([N+](=O)[O-])cc12]

### General Procedure for Preparation of trinitro-9,10-[1,2]benzenoanthracene-9(10H)-carboxylic acid (6a-6c)

To 1 eq of 5a (or 5b, 5c) dissolved in p-dioxane was added 3 eq of 1M NaOH (aq) and heated to 60° C. for 24 h. After the reaction was completed, the solution was neutralized and acidified with 1N HCl. Ethyl acetate was added to the solution and the organic layer was extracted from the solution. The combined organic layer was washed with NH₄Cl (aq) and brine. The organic layer was dried with anhydrous sodium sulfate, and concentrated in vacuo to give 6a (or 6b, 6c) in quantitative yield.

### 2,7,15-trinitro-9,10-[1,2]benzenoanthracene-9(10H)-carboxylic acid (6a)

Physical Property: Pale yellow solid, m.p.=358-359° C.

TLC: R_{f}=0.43 (silica gel, 100% ethyl acetate).

¹H NMR (500 MHz, (CD₃)₂CO) δ 9.23 (s, 3H), 7.96 (dd, 3H, J=8.1, 1.9 Hz), 7.74 (d, 3H, 8.1 Hz), 6.21 (s, 1H).

¹³C NMR (125 MHz, (CD₃)₂CO) δ 171.3, 151.3, 147.5, 146.1, 124.6, 121.8, 121.4, 63.7, 53.4.

IR (neat): 2924, 1592, 1518, 1341, 1262, 1092, 1069, 1023, 903 cm⁻¹.

HRMS (ESI) calculated for C₂₁H₁₀N₃O₈ ⁻[M−H]⁻ 432.0473, found 432.0457.

### (9s,10r)-2,6,15-trinitro-9,10-[1,2]benzenoanthracene-9(10H)-carboxylic acid (6b)

Physical Property: Pale yellow solid, m.p.=265-266° C.

TLC: R_{f}=0.23 (silica gel, 100% ethyl acetate).

¹H NMR (500 MHz, (CD₃)₂CO) δ 9.29 (d, 2H, J=2.2 Hz), 8.54 (d, 1H, J=8.3 Hz), 8.36 (d, 1H, J=2.2 Hz), 8.01 (dd, 2H, J=8.1, 2.2 Hz), 7.88 (d, 1H, J=8.1 Hz), 7.81 (d, 2H, J=8.1 Hz), 6.28 (s, 1H).

¹³C NMR (125 MHz, (CD₃)₂CO) δ 171.2, 152.5, 151.7, 146.8, 146.0, 145.9, 145.5, 127.3, 124.6, 121.8, 121.4, 121.1, 118.6, 63.7, 53.1.

IR (neat): 2921, 1737, 1593, 1524, 1462, 1377, 1344, 1260, 1093 cm⁻¹.

HRMS (ESI) calculated for C₂₁H₁₀N₃O₈ [M−H]⁻ 432.0473, found 432.0462.

### (9r,10s)-2,6,14-trinitro-9,10-[1,2]benzenoanthracene-9(10H)-carboxylic acid (6c)

Physical Property: White solid, m.p.=202-203° C.

TLC: R_{f}=0.03 (silica gel, 100% ethyl acetate).

¹H NMR (500 MHz, (CD₃)₂CO) δ 8.92 (s, 1H), 8.49 (s, 2H), 8.23 (d, 2H, J=8.6 Hz), 8.15-8.06 (m, 3H), 7.95 (d, 1H, 8.2 Hz), 6.47 (s, 1H).

¹³C NMR (125 MHz, (CD₃)₂CO) δ 168.6, 151.1, 148.2, 146.2, 146.1, 145.7, 142.9, 125.8, 125.53, 122.51, 121.7, 120.1, 119.5, 61.8, 52.5.

IR (neat): 2927, 1720, 1598, 1518, 1458, 1418, 1341, 1260, 1179, 1090, 902 cm⁻¹.

HRMS (ESI) calculated for C₂₁H₁₀N₃O₈ [M−H]⁻ 432.0473, found 432.0466.

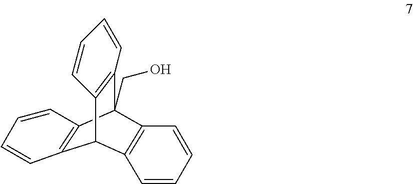 [SMILES: OCC12c3ccccc3C(c3ccccc31)c1ccccc12]

### 9,10-[1,2]benzenoanthracen-9(10H)-ylmethanol (7)

To a vial was added 1.95 g (5.94 mmol) of 4, 70 mL of 1M HCl, and 100 mL of THF. The solution was stirred at 25° C. After 1 h, ethyl acetate was added to the solution. The organic layer was extracted from the solution, dried with anhydrous sodium sulfate, and concentrated in vacuo to give 7 (1.68 g, 99% isolated yield).

Physical Property: Pale yellow solid.

TLC: R_{f}=0.31 (silica gel, 25% ethyl acetate/hexanes).

¹H NMR (500 MHz, (CD₃)₂CO) δ 7.65-7.44 (m, 6H), 7.04-6.97 (m, 6H), 5.58 (s, 1H), 5.30 (d, 2H, J=3.6 Hz), 4.60 (t, 1H, J=3.6 Hz).

¹³C NMR (125 MHz, (CD₃)₂CO) δ 147.2, 145.6, 124.71, 124.66, 123.3, 122.9, 60.0, 54.2, 54.0.

IR (neat): 3308, 2962, 2918, 1579, 1458, 1261, 1071, 1035, 798, 740, 648, 629, 611, 481 cm⁻¹.

HRMS (ESI) calculated for C₂₁H₁₇O⁺ [M+H]⁺ 285.1274, found 285.1288.

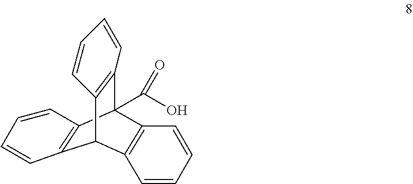 [SMILES: O=C(O)C12c3ccccc3C(c3ccccc31)c1ccccc12]

### 9,10-[1,2]benzenoanthracene-9(10H)-carboxylic acid (8)

To a vial, 31.9 mg (0.11 mmol) of 7 was dissolved in acetone and heated to 50° C. 88.6 mg (0.56 mmol) of KMnO₄ was added to the solution. Whenever the solution turned to black or brown, an additional 88.6 mg (0.56 mmol) of KMnO₄ was added to the solution. After 3 days, sodium sulfite solution (aq) was added to the crude mixture and then extracted with ethyl acetate. The combined organic layer was dried with anhydrous sodium sulfate, and concentrated in vacuo to yield 8 (22.4 mg, 67% isolated yield).

Physical Property: Pale yellow solid.

TLC: R_{f}=0.28 (silica gel, 100% ethyl acetate).

¹H NMR (500 MHz, (CD₃)₂CO) δ 8.09-8.01 (m, 3H), 7.46-7.42 (m, 3H), 7.02-6.97 (m, 6H), 5.57 (s, 1H)

¹³C NMR (125 MHz, (CD₃)₂CO) δ 172.3, 146.4, 144.6, 125.1, 124.6, 124.6, 123.2, 62.4, 54.2.

IR (neat): 2925, 1712, 1458, 1448, 1386, 1261, 1213, 1171, 1085, 1032, 867, 801, 748, 735, 703, 685, 645, 624, 609, 478 cm⁻¹.

HRMS (ESI) calculated for C₂₁H₁₃O₂⁻ [M−H]⁻ 297.0921, found 297.0914.

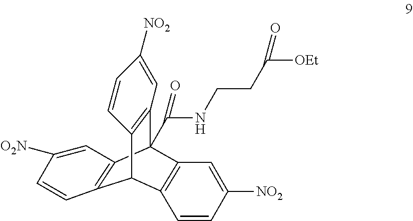 [SMILES: CCOC(=O)CCNC(=O)C12c3cc([N+](=O)[O-])ccc3C(c3ccc([N+](=O)[O-])cc31)c1ccc([N+](=O)[O-])cc12]

### ethyl 3-(2,7,15-trinitro-9,10-dihydro-9,10-[1,2]benzenoanthracene-9-carboxamido)propanoate (9)

To a round bottom flask was added 213.2 mg (0.49 mmol) of 6a, 97.3 mg (1.23 mmol) of pyridine, and 10 mL of CH₂Cl₂. 140.9 mg (1.23 mmol) of MsCl was added to the solution at 0° C. After 30 minutes, 188.9 mg (1.23 mmol) of betaalanine ethyl ester hydrochloride and 97.3 mg (1.23 mmol) of pyridine in 10 mL of CH₂Cl₂ was added to the solution and warmed to 25° C. After 1 h, the solution was dried in vacuo and triturated with ethyl acetate several times to give 9 (240 mg, 91% isolated yield).

Physical Property: White solid, m.p.=304-305° C.

TLC: R_{f}=0.23 (silica gel, 50% ethyl acetate/hexanes).

¹H NMR (500 MHz, (CD₃)₂CO) δ 8.92 (d, 3H, J=2.2 Hz), 8.19-8.12 (bs, 1H), 8.09 (dd, 3H, J=8.2, 2.2 Hz), 7.90 (d, 3H, J=8.2 Hz), 6.40 (s, 1H), 4.19 (q, 2H, J=7.2 Hz), 4.14-4.08 (m, 2H), 2.99 (t, 2H, J=6.6 Hz), 1.25 (t, 3H, J=7.2 Hz).

¹³C NMR (125 MHz, (CD₃)₂CO) δ 171.3, 166.6, 150.5, 146.1, 144.5, 125.5, 122.4, 120.4, 60.2, 60.0, 53.1, 35.9, 33.6, 13.6.

IR (neat): 3301, 2924, 1722, 1668, 1521, 1342, 1261, 1203, 1031, 800 cm⁻¹.

HRMS (ESI) calculated for C₂₆H₂₀N₄NaO₉⁺ [M+Na]⁺ 555.1122, found 555.1127.

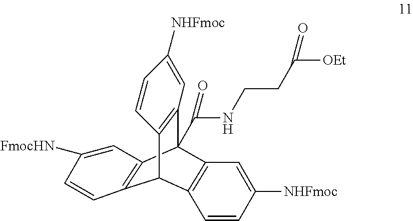

### ethyl 3-(2,7,15-tris(4(9H-fluoren-9-yl)methoxy)carbonyl)amino)-9,10-dihydro-9,10-[1,2]benzenoanthracene-9-carboxamido)propanoate (11)

To a vial was charged 180 mg (0.338 mmol) of 9, 3.6 mg (0.034 mmol) of Pd/C, 2 mL of MeOH under H₂ gas. After 24 h stirring at 25° C., the solution was filtered and concentrated in vacuo to yield crude of 10. To a round flask was added 10, 0.27 mL (3.38 mmol) of pyridine, and 30 mL of CH₂Cl₂. The solution was cooled and stirred for 30 minutes at 0° C. 612.1 mg (2.37 mmol) of Fmoc-Cl in 3 mL of CH₂Cl₂ was added to the solution. The crude mixture was warmed to 25° C. and stirred for 16 h. The solution was washed with saturated NH₄Cl(aq), dried, and purified by column chromatography using ethyl acetate (100%) as the eluent to give 11 (310 mg, 83% isolated yield).

Physical Property: White solid, m.p.=157-158.3° C.

TLC: R_{f}=0.81 (silica gel, 50% ethyl acetate/hexanes).

¹H NMR (500 MHz, CD₂Cl₂) δ 7.98 (s, 3H), 7.78 (d, 6H, J=7.5 Hz), 7.59 (d, 6H, J=6.3 Hz), 7.39 (t, 6H, J=7.5 Hz), 7.32-7.20 (m, 12H), 7.15 (s, 3H), 6.75 (bs, 1H), 5.28 (s, 1H), 4.40 (d, 6H, J=6.7 Hz), 4.19 (t, 3H, J=6.7 Hz), 4.06 (q, 2H, J=7.1 Hz), 3.89 (q, 2H, J=5.7 Hz), 2.80 (t, 2H, J=5.7 Hz), 1.11 (t, 3H, J=7.1 Hz).

¹³C NMR (125 MHz, CD₂Cl₂) δ 173.1, 168.8, 153.4, 144.3, 143.9, 141.4, 141.3, 135.2, 127.7, 127.0, 125.0, 123.6, 119.9, 115.9, 66.7, 60.9, 60.5, 52.4, 47.1, 35.8, 34.1, 13.9.

IR (neat): 1715, 1604, 1526, 1464, 1450, 1409, 1322, 1297, 1260, 1213, 1155, 1055, 985, 804, 758, 737, 702, 621, 531, 501 cm⁻¹.

HRMS (ESI) calculated for C₇,H₅₆N₄NaO₉+[M+Na]⁺ 1131.3940, found 1131.3949.

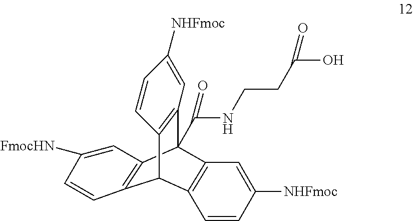

### 3-(2,7,15-tris(4(9H-fluoren-9-yl)methoxy)carbonyl)amino)-9,10-dihydro-9,10-[1,2]benzenoanthracene-9-carboxamido)propanoic acid (12)

To a vial was charged 30.0 mg (0.027 mmol) of 11, 0.1 mL of H₂SO₄, 4 mL of 1,4-dioxane, and 2 mL of water. The solution was stirred at 80° C. After 24 h, the solution was concentrated in vacuo and purified by column chromatography using ethyl acetate (100%) as the eluent to give 12 in quantitative yield.

Physical Property: White solid, m.p.=188-189° C.

TLC: R_{f}=0.67 (silica gel, 100% ethyl acetate).

¹H NMR (500 MHz, (CD₃)₂SO) δ 9.83-9.53 (bs, 3H), 8.12-7.94 (bs, 4H), 7.87 (d, 6H, J=7.5 Hz), 7.71 (d, 6H, J=7.5 Hz), 7.39 (t, 6H, J=7.5 Hz), 7.30 (t, 6H, J=7.5 Hz), 7.28-7.07 (m, 6H), 5.36 (s, 1H), 4.39 (d, 6H, J=6.8 Hz), 4.25 (t, 3H, J=6.8 Hz), 3.78-3.68 (m, 2H), 2.78-2.62 (m, 2H).

IR (neat): 3324, 2924, 1719, 1604, 1536, 1464, 1299, 1216, 1052, 985 cm⁻¹.

HRMS (ESI) calculated for C₆₉H₅₁N₄O₉⁻ [M−H]⁻ 1079.3662, found 1079.3630.

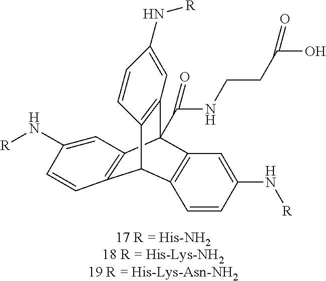

### General Procedure for Preparation of 17-19 (Solid-Phase Peptide Synthesis)

To a SPPS reaction vessel was added 1 eq of 2-chlorotrityl chloride resin (100-200 mesh, substitution: 1.4 mmol/g). The resin was stirred in dry CH₂Cl₂ for 30 min and the solvent was removed by vacuum. 1.2 eq of 12 dissolved in dimethylformamide:CH₂Cl₂ (1:5 volume ratio) and 5 eq of N,N-diisopropylethylamine (DIPEA) were added to the resin. After stirring for 10 min, an additional 1.5 eq of DIPEA was added to the resin and stirred overnight (12 hours) at 25° C. HPLC grade methanol was added and stirred for 20 min to cap the remaining reactive functional group on the resin. The solution was removed by vacuum and the resin was washed with CH₂Cl₂ (1 min, 3 times) and dimethylformamide (1 min, 3 times). 20% (v/v) piperidine in dimethylformamide was added to the resin, stirred for 1 h, and then the solution was drained. The resin was washed with dimethylformamide (1 min, 3 times), CH₂Cl₂ (1 min, 3 times) and dimethylformamide (1 min, 3 times). 9.5 eq of corresponding Fmoc-protected amino acid (Fmoc-His(trt)-OH, Fmoc-Lys(boc)-OH, or Fmoc-Asn(trt)-OH) was pre-activated with 9 eq of HATU and 18 eq of DIPEA in dimethylformamide. The pre-activated solution was then added to the reaction vessel and stirred for ˜12 hours overnight. The solution was removed by vacuum and the resin was washed with dimethylformamide (1 min, 3 times), CH2Cl2 (1 min, 3 times) and dimethylformamide (1 min, 3 times). 20% (v/v) piperidine in dimethylformamide was added to the resin, stirred for 1 h, and then the solution was drained. The process of washing the resin and the amino acid coupling was repeated until the desired sequence of peptide was achieved. When the peptide coupling is completed, the resin was washed with dimethylformamide (1 min, 4 times), CH₂Cl₂ (1 min, 4 times). The desired product was cleaved from the resin by treating a mixture of trifluoroacetic acid (TFA), 2,2,2-trifluoroethanol (TFE), and CH₂Cl₂ (9:1:1 volume ratio) for 30 min twice. For compound 19, cleavage took 12h. The cleavage solution was then collected and concentrated in vacuo. The crude mixture was dissolved in MilliQ water and purified by reverse-phase HPLC. Purified products (17-19) were analyzed by MALDI-MS and analytical reverse-phase HPLC for the purity.

HPLC analysis of compound 5a-5c:

After the nitration reaction of 4, 7, and 8 with nitric acid for 24 h at 80° C., the crude mixture was cooled, neutralized with K2CO3, and re-acidified with 1M HCl. Ethyl acetate was added to the solution and the organic layer was extracted from the solution. The combined organic solution was dried with anhydrous sodium sulfate, and concentrated in vacuo. Crude mixtures were dissolved in acetonitrile. For quantitative analysis, 9,10-diphenylanthracene (internal standard) dissolved in acetonitrile was added to the crude mixture. All samples were then analyzed by reverse-phase HPLC. Solvent gradient method used is shown below. (A: 0.1% CF₃CO₂H in MilliQ water, B: Methanol).

HPLC analysis of compound 12 and 17-19: the purified samples were dissolved in acetonitrile (for compound 12) or in MilliQ water (for compounds 17-19) and then analyzed by reverse-phase HPLC to confirm the purity of samples. Two different gradients were used as shown below (left: compound 12, right: compound 17-19, A: 0.1% CF₃CO₂H in MilliQ water, B: Acetonitrile).

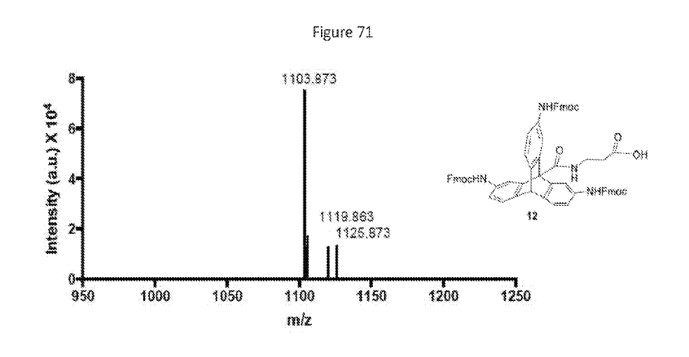

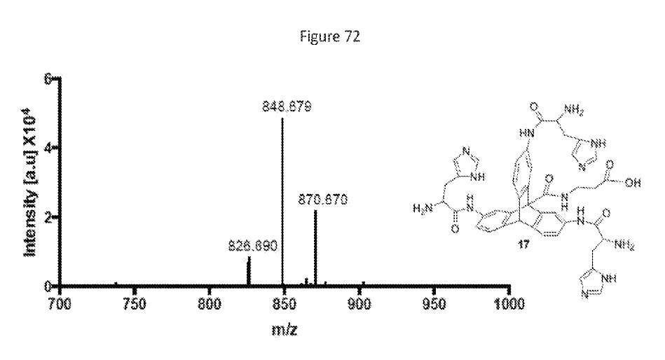

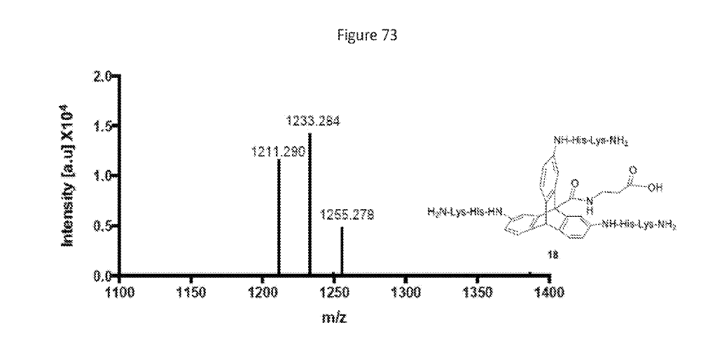

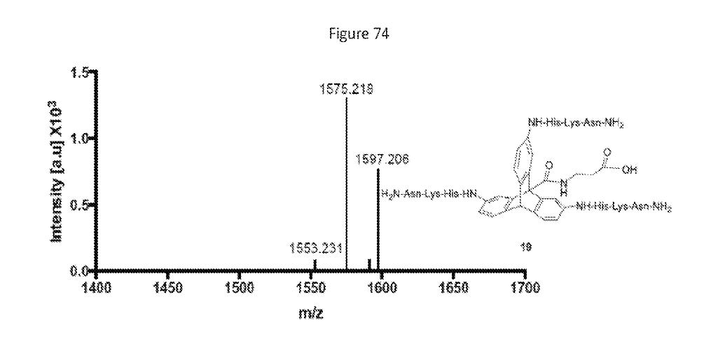

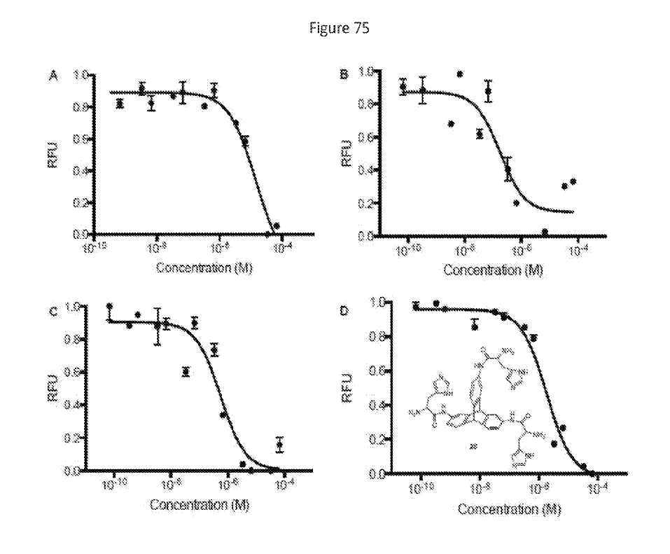

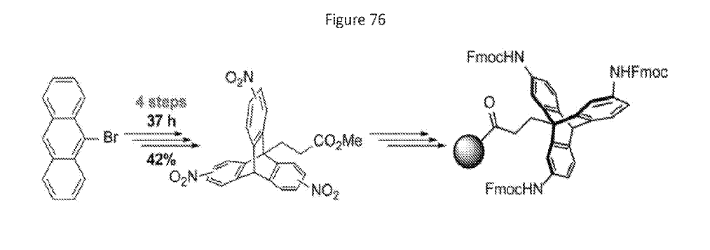

MALDI-MS analysis: MALDI-MS data of compounds 12, 17, 18 and 19 are shown in FIGS. 71-74 respectively.

Fluorescence-quenching experiments were conducted in 50 mM sodium phosphate buffer, pH 7.2. Inhibitor (I10) strand binding curves were obtained by adding 1 μL of increasing concentrations of I10 to 19 μL of 120 nM FQTNR 3WJ. Samples were incubated for 2 hours and ran in triplicate. Inhibitor strand displacement curves with tripycenes were obtained by incubating 14 μL of 120 nM FQ-TNR 3WJ with 1 μL of 150 μM I10 for 2 hours a room temperature. To this complex, 1 μL of increasing concentrations of triptycene was added and incubated for 2 hours. Fluorescence measurements were conducted in a 384-well plate and were recorded with an excitation at 495 nm and emission at 520 nm using 5 nm bandwidths.

### Example 5

### Synthesis Bridgehead-Substituted Triptycenes for Discovery of Nucleic Acid Junction Binders

Figures, tables and references are as published in Barros et al., Organic Letters, 2016(18):2423, published on May 12, 2016.

Recently, the utility of triptycene as a scaffold for targeting nucleic acid three-way junctions was demonstrated. A rapid, efficient route for the synthesis of bridgehead-substituted triptycenes is reported, in addition to solid-phase diversification to a new class of triptycene peptides. The triptycene peptides were evaluated for binding to a d(CAG)ₙ(CTG) repeat DNA junction exhibiting potent affinities. The bridgehead-substituted triptycenes provide new building blocks for rapid access to diverse triptycene ligands with novel architectures.

Nucleic acid junctions are important structural intermediates in biology. Junctions are present in important biological processes including replication. These junctions also occur in viral genomes in addition to trinucleotide repeat expansions associated with numerous neurodegenerative diseases. These structures are also present in nanostructures and aptamer-based sensors. The ability to selectively modulate a subset of nucleic acid structures using small molecules would allow for the chemical control of cellular processes as well as the reprogramming of cellular events. The ability to differentially stabilize predefined nucleic acid structures or to reprogram and bias the equilibrium distribution of an ensemble of structures in a precise manner could have a profound impact not only in biology but also in nucleic acid nanotechnology and materials applications.

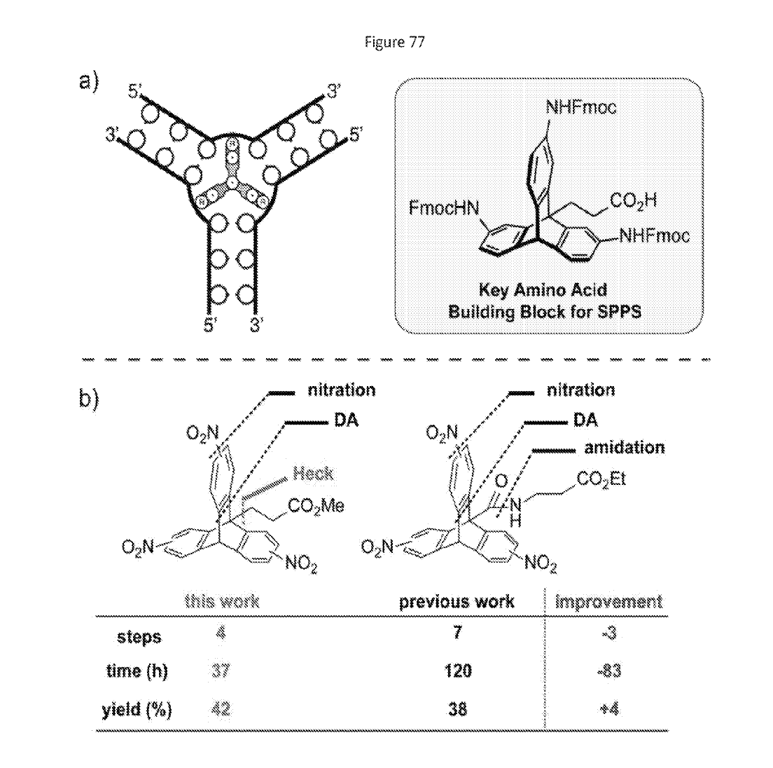

We previously demonstrated that triptycene-based molecules can bind to three-way junctions (3WJs). Additionally, we have shown that these molecules bind to d(CAG)⋅(CTG) repeats implicated in triplet repeat expansion diseases. The ability to synthesize libraries of triptycene derivatives on solid supports will accelerate efforts to identify biologically relevant nucleic acid junction binders and provide further insight into the molecular recognition properties of triptycenes toward diverse junction sequences and topologies. To facilitate solidphase immobilization, a point of attachment on triptycene is required. The bridgehead position provided a strategic location, as it is equidistant from the three amino groups that serve as sites of diversification (FIG. 77a). We recently described a synthesis for bridgehead-substituted triptycene building blocks in Example 4. Here, a modified, more efficient synthesis by utilizing a combined Heck coupling/benzyne Diels-Alder strategy is disclosed. The new triptycene building block is further diversified on solid phase with short di- and tripeptides, and the final compounds are evaluated for binding to a d(CAG)ₙ(CTG) repeat junction. New high-affinity lead compounds for this junction motif that will form the basis of further investigations are discovered.

Similar to our previous route, our synthetic plan relied on the reduction of nitrated triptycene, a key intermediate, to install the three key amine functional groups that serve as points of future diversification (FIG. 77b). The synthetic strategy presented here provides a shorter synthesis with only four steps to the key intermediate compared to seven steps in our previous route. Additionally, this method significantly reduced total reaction times from 120 to 37 h and showed an improvement in overall yield (FIG. 77b). Moreover, the solubility of intermediates was improved. After extending the linker at the bridgehead via an amidation reaction in the previous route, the resulting product showed poor to moderate solubility in most organic solvents. However, the intermediates in this synthetic route have good solubility, allowing easier characterization and large-scale reactions. In addition, a new regioisomer 5c that has all three nitro groups facing away from the linker was isolated in this new synthetic route, whereas this regioisomer was not observed in the previous report.

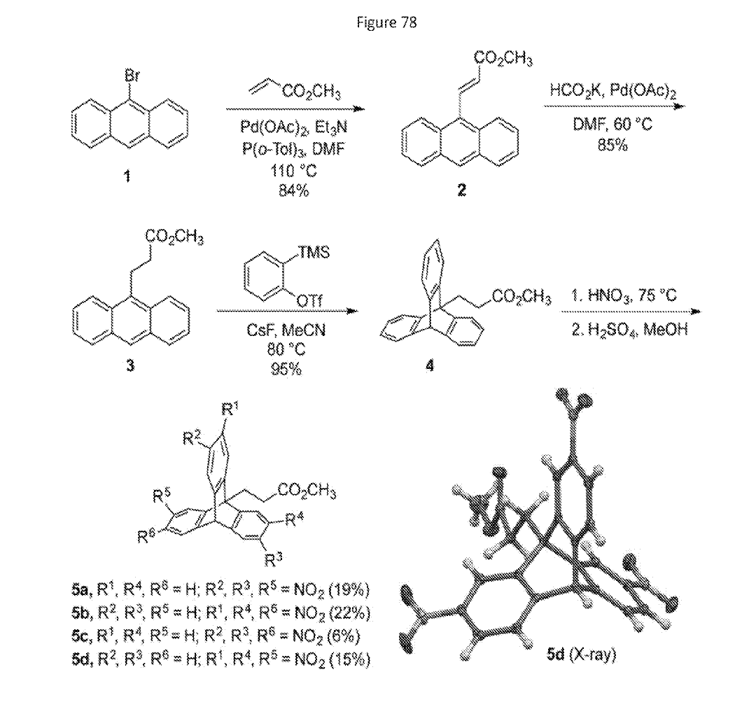

We initiated our synthesis with a Heck reaction between 9-bromoanthracene 1 and methyl acrylate in the presence of palladium (II) acetate, tri-o-tolylphosphine, and triethylamine in a sealed tube. The Heck reaction proceeded cleanly and resulted in the desired product 2 in 84% yield (FIG. 78).

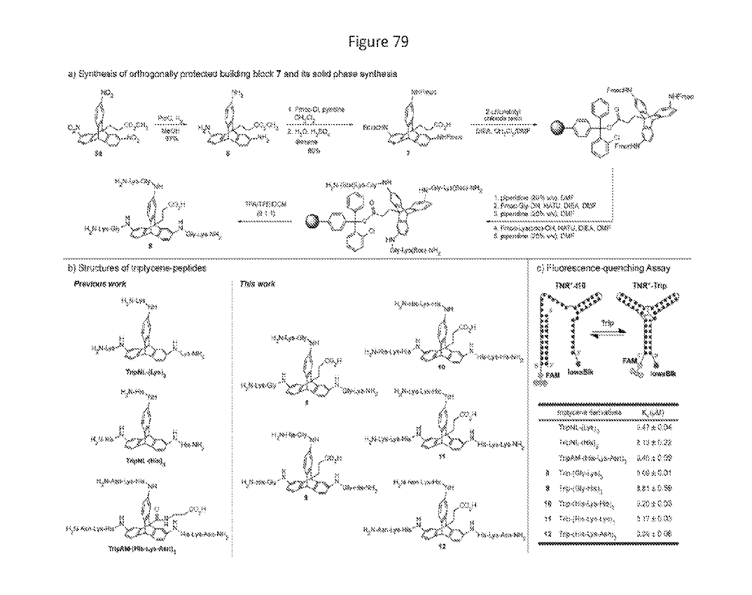

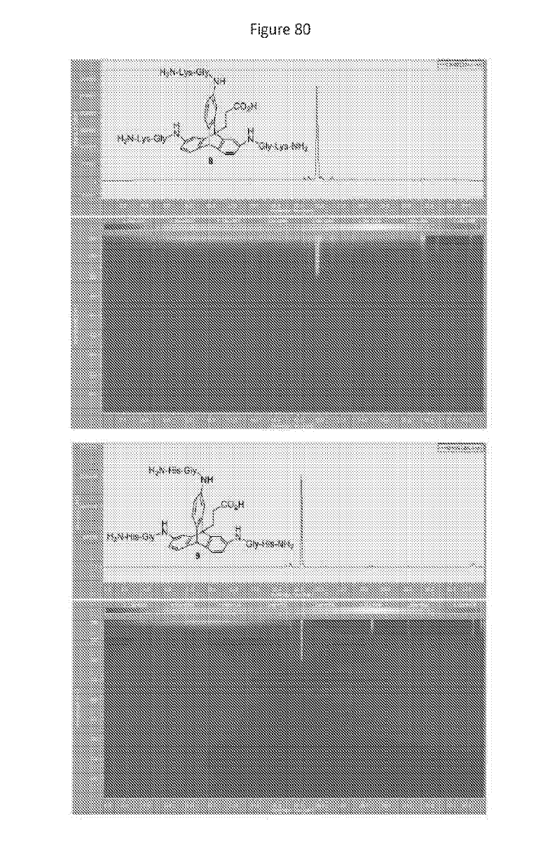

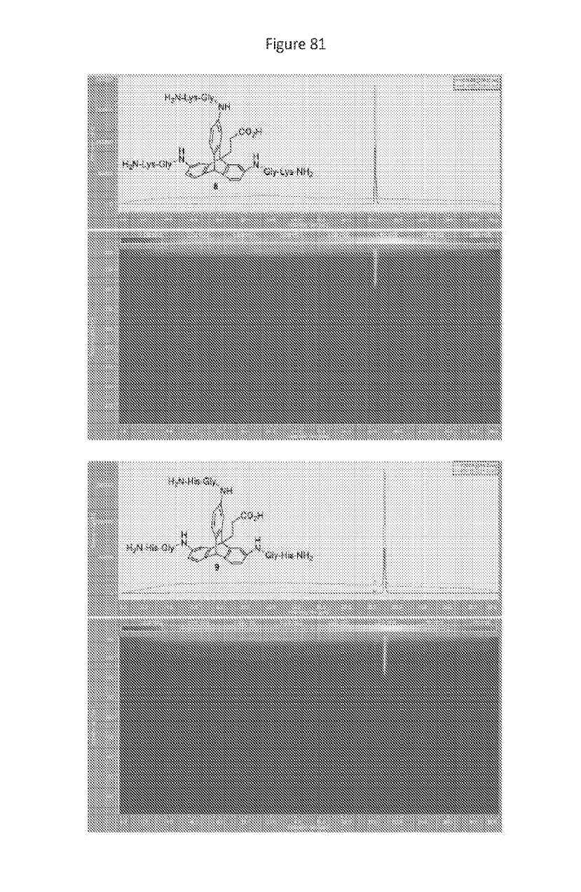

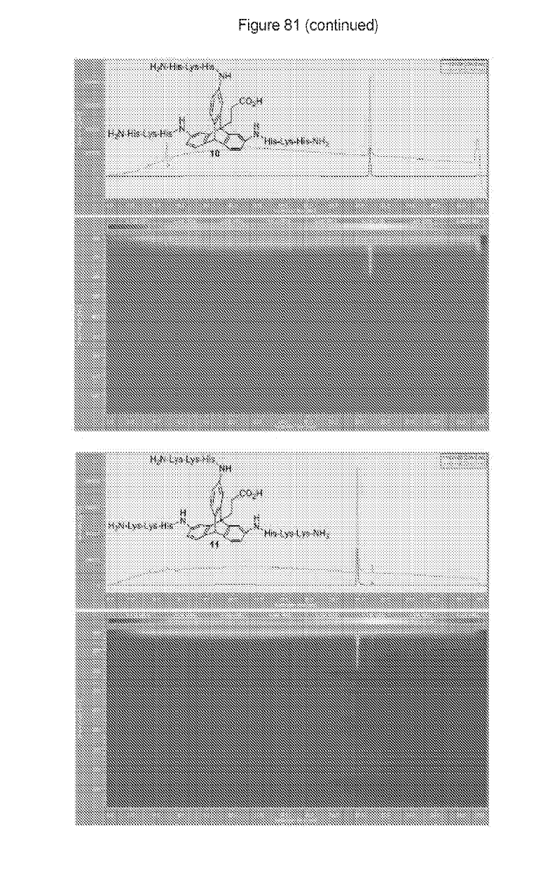

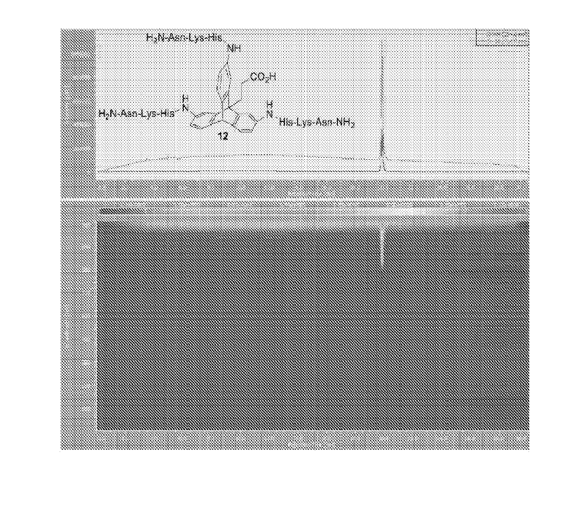

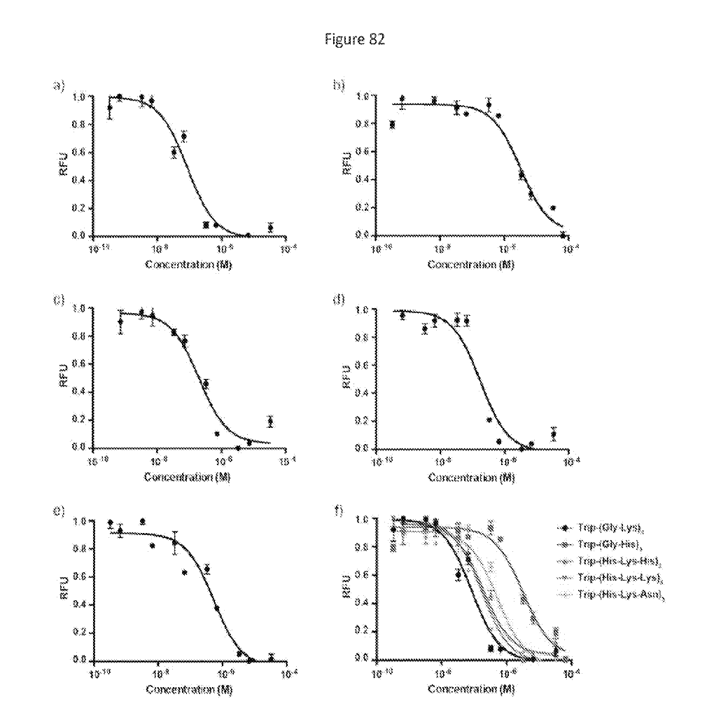

Next, olefin 2 was reduced under mild conditions using palladium (II) acetate as the catalyst and potassium formate as the hydrogen source, producing 3 in 85% yield. The key Diels-Alder reaction with anthracene 3 and benzyne, generated in situ from 2-(trimethylsilyl)phenyl trifluoromethanesulfonate and cesium fluoride, proceeded smoothly to yield bridgeheadsubstituted triptycene 4 in 95% yield. Nitration of triptycene resulted in hydrolysis of the bridgehead ester and four major nitrated regioisomers that proved inseparable by standard chromatographic techniques. Esterification of the crude reaction greatly facilitated the separation of the regioisomeric mixture (5a-d) using standard silica gel column chromatography. The nitrated triptycene regioisomers were characterized by HMBC and HSQC. A crystal of triptycene 5d was obtained in chloroform to confirm its structure by X-ray crystallography (FIG. 79).

Next, isomer 5d was utilized in subsequent transformations that were described in the previous publication. Pd/C-catalyzed hydrogenation, Fmoc protection, and acid-catalyzed hydrolysis of the ester were performed to yield protected triptycene acid 7 in 78% yield over three steps. A key building block 7 was immobilized on 2-chlorotrityl chloride resin in preparation for solid-phase diversification (FIG. 79a). After addition of triptycene and washing of the resin, the Fmoc groups on triptycene were deprotected using piperidine in DMF (20% v/v) for 1 h. A decreased reaction time led to incomplete deprotection of all three Fmoc groups. After deprotection, the first amino acid was coupled onto the immobilized triptycene using HATU and DIEA. Overnight couplings were required for complete reaction with all three hindered aniline nitrogens. Next, subsequent deprotections followed by coupling of the desired amino acids were continued until the final sequence was obtained. The final deprotection of the amino acid side chain protecting groups and cleavage from resin were performed simultaneously using 9:1:1 TFA/TFE/DCM. The resulting triptycene peptides were purified by reversed phase HPLC and characterized prior to evaluation of the junction binding properties. In this manuscript, we focused our efforts on mono-, di-, and tripeptides to maximize diversity while maintaining minimal molecular weight. Longer peptides can certainly be produced although cell permeability will be a consideration as the size increases.

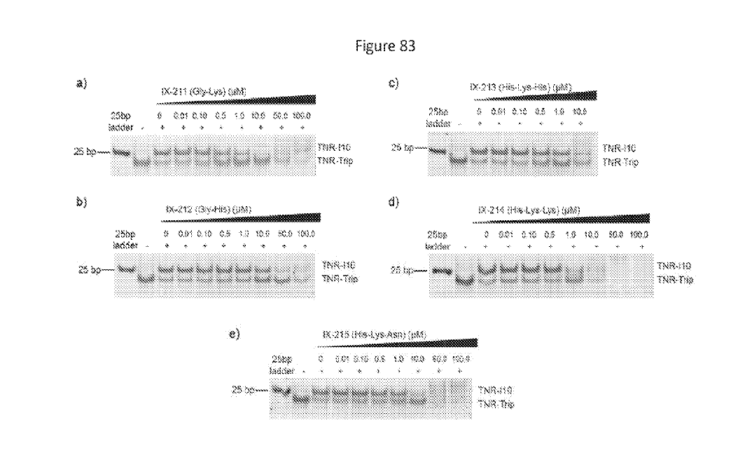

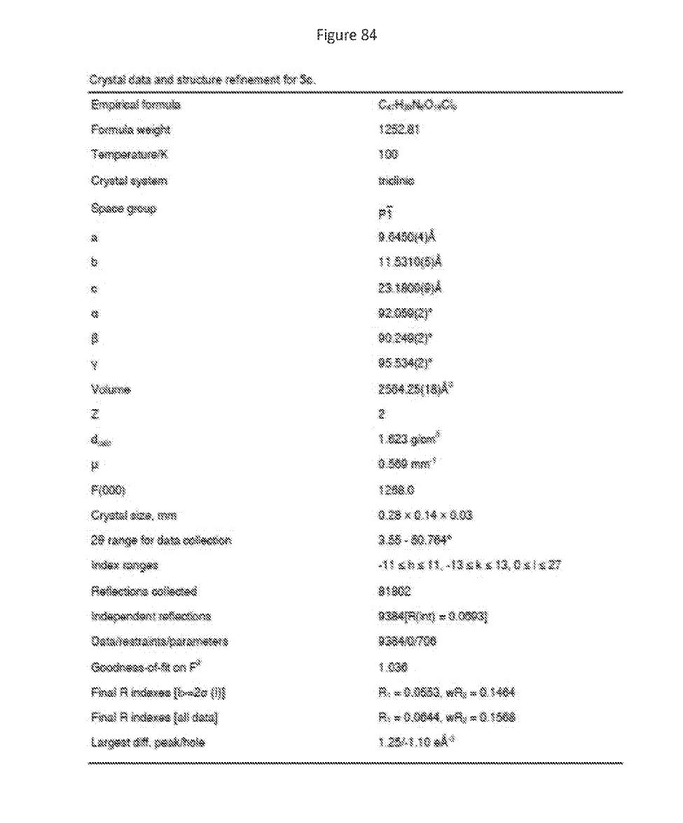

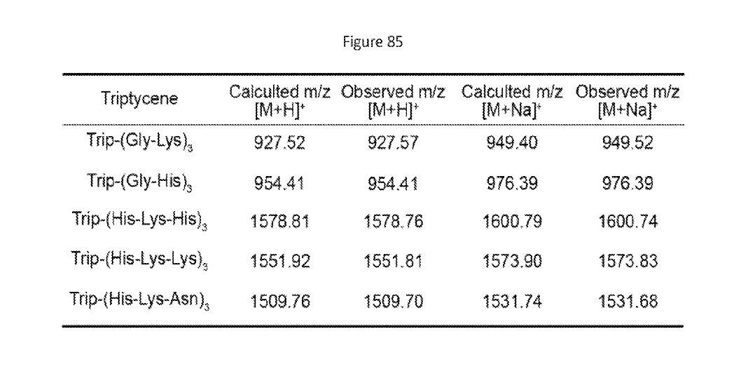

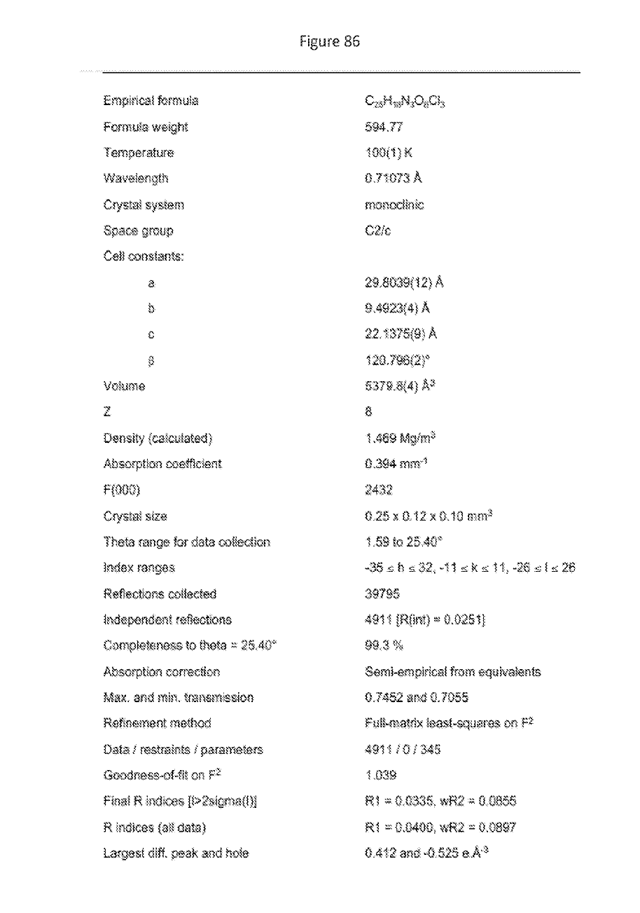

Binding of the amino acid substituted triptycenes was evaluated against a slipped-out d(CAG)ₙ(CTG) repeat nucleic acid junction. Lysine and histidine containing triptycenes were synthesized due to their large presence in nucleic acid-protein interfacial interactions. Among the molecules previously tested, TripNL-(Lys)₃ and TripNL-(His)₃ exhibited the highest affinity toward the junction. Several dimeric and trimeric amino acid substituents were synthesized for comparison (FIG. 79b). A high-throughput assay in which the 3WJ was labeled with a fluorophore and a quencher was used to determine binding. The addition of a 10 bp oligonucleotide strand that was complementary to the 5′ end of the junction (I10) opened the structure, resulting in a highly fluorescent state (TNR*-I10), as shown in FIG. 79c. Titration of junction-stabilizing molecules resulted in quenching of fluorescence due to displacement of the inhibitor strand and reformation of the junction (TNR*-Trip). To determine if increased flexibility of the amino acid may play an important role in binding, glycine was coupled directly to the triptycene core followed by lysine or histidine. Trip-(Gly-Lys)₃ (8) exhibited increased potency compared to that of Trip-(Lys)₃, with a Kd of 90 nM, indicating that the increased flexibility may allow for better binding. This triptycene derivative demonstrates the highest binding affinity toward the TNR junction thus far. Interestingly, Trip-(Gly-His)₃ (9) did not exhibit improved binding compared to that of Trip-(His)₃. Triptycenes substituted with three amino acids were also synthesized using lysine, histidine, and asparagine. Trip-(His-Lys-His)₃ (10), Trip-(His-Lys-Lys)₃ (11), and Trip-(His-Lys-Asn)₃ (12), which only differ in their final amino acid, exhibited Kd values of 0.20, 0.17, and 0.39 μM, respectively. It should be noted that most triptycene derivatives synthesized in this work showed improved binding affinity compared to the most potent triptycene derivative from the previous work, which exhibited a K_{d} value of 0.27 μM. We also compared the binding affinity of Trip-(His-Lys-Asn)₃ (12) to that of TripAM-(His-Lys-Asn)₃, which have the same peptide sequence but an amide linker at the bridgehead. They exhibited similar binding affinities toward the junction. Triptycenes 8-12 were also characterized using a gel shift assay, where the inhibitor strand was incubated with unlabeled 3WJ (see Supporting Information). This change resulted in an electrophoretic shift that is consistent with a larger complex. Titration of triptycene with this complex resulted in reformation of the nucleic acid junction (FIG. 83).

In summary, we have developed a shorter, more efficient synthetic strategy toward a bridgehead-substituted triptycene building block. This new synthetic route is improved in terms of solubility, enabling large-scale reactions. Moreover, this route provides an interesting new regioisomer that was not observed through the previous route. A building block with an attachment point at the bridgehead provided rapid access to new triptycene peptide derivatives using solid-phase synthesis methods. The triptycene peptides were evaluated for nucleic acid junction binding to a triplet repeat expansion oligonucleotide using a fluorescence-based assay, which revealed the most potent binder to this junction to date. New triptycene building blocks that are amenable to solid-phase diversification provide a path for the discovery of new junction binders with superior properties. This new class of bridgehead-substituted triptycenes may allow for the generation of one-bead-one-compound combinatorial libraries for the rapid discovery of new junction binders using fluorescently labeled junctions. Additionally, this new class of bridgehead-substituted triptycenes opens the door for the creation of pull-down probes to identify cellular targets in future studies.

General Materials:

All commercial reagents and solvents were used as received. 9-bromoanthracene, potassium formate, nitric acid, Fmoc chloride, pyridine, and acetonitrile were purchased from Sigma-Aldrich (St. Louis, Mo.). Methyl acrylate, triethylamine (Et₃N), tri-o-tolylphosphine, palladium(II) acetate, cesium fluoride, and Pd/C were purchased from Acros Organics. Methanol, dichloromethane (DCM), dimethylformamide (DMF) were purchased from Fisher Scientific (Waltham, Mass.). (1-[Bis(dimethylamino)methylene]-1H-1,2,3-triazolo[4,5-b]pyridinium3-oxide hexafluorophosphate) (HATU) was purchased from Oakwood Products, Inc. (West Colombia, S.C.), 2-chlorotrityl chloride resin was purchased from Advanced ChemTech (Louisville, Ky.), diisopropylethylamine (DIEA), trifluoroacetic acid (TFA), and 2,2,2-trifluoroethanol (TFE) were purchased from Alfa Aesar (Ward Hill, Mass.), and piperidine was purchased from American Bioanalytical (Natick, Mass.). Chloroform-d, methanol-d4, dimethylsulfoxide-d6 were purchased from Cambridge Isotope Laboratories (Tewksbury, Mass.). Thin-layer chromatography was done using Sorbent Technologies (Norcross, Ga.) silica plates (250 μm thickness). Flash chromatography was performed on a Teledyne Isco (Lincoln, Nebr.) CombiFlash R_{f} system using RediSep R_{f} silica columns.

TNR DNA 3WJ (5′-GCGGAGCAGCCCTTGGGCAGCACCTTGGTGCTGCTCCGC-3′) and DNA inhibitor 10 (5′-GCTGCTCCGC-3′) were purchased from Integrated DNA Technologies (IDT). HPLC-purified TNR DNA 3WJ oligo modified with a 5′-FAM and a 3′-IowaBlack was purchased from IDT.

¹H and ¹³C NMR were recorded on a Bruker UNI 500 NMR at 500 and 125 MHz, respectively. High resolution mass spectra were obtained at the University of Pennsylvania Mass Spectrometry Center on a Waters LC-TOF mass spectrometer (model LCT-XE Premier) using electrospray ionization in positive or negative mode, depending on the analyte. High performance liquid chromatography was performed on a JASCO HPLC (Easton, Md.) equipped with a Phenomenx (Torrance, Calif.) column (Analytical: Luna 5μ, C18(2) 100 A; 250×4.60 mm, 5 μm Semi-prep: 5μ C18(2) 100 A; 250×10.00 mm, 5 μm) using aqueous (H₂O+0.1% CF₃CO₂H) and organic (CH₃CN) phases. Matrix-assisted laser desorption ionization (MALDI) mass spectra were obtained on a Bruker Ultraflex III MALDI-TOF-TOF mass spectrometer (Billerica, Mass.) using α-cyano-4-hydroxycinnamic acid (CHCA). Fluorescence measurements were collected on a Tecan M1000 plate reader (Mannedorf, Switzerland).

Synthesis:

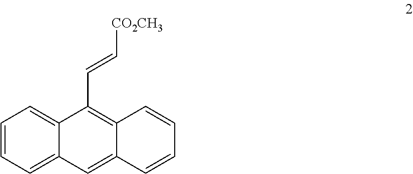 [SMILES: COC(=O)/C=C/c1c2ccccc2cc2ccccc12]

### methyl-3-(anthracen-9-yl)acrylate (2)

A solution of 9-bromoanthracene (192 mg, 0.746 mmol), methyl acrylate (642 mg, 7.46 mmol), Et3N (755 mg, 7.46 mmol), tri-o-tolylphosphine (25 mg, 0.082 mmol), and Pd(OAc)2 (8.37 mg, 0.0373 mmol) in DMF (7 mL) was heated at 120° C. in a sealed tube for 5 h. Upon cooling, the mixture was filtered through Celite and washed with ethyl acetate. The filtrate was extracted with ethyl acetate and water several times. Combined organic layers were then dried over Na₂SO₄. The crude mixture was purified by column chromatography on silica gel (5% EtOAc/hexanes) to give 1 (164 mg, 84%). ¹H NMR (500 MHz, CDCl3) δ 8.65 (d, 1H, J=16.3 Hz), 8.45 (s, 1H), 8.25-8.23 (m, 2H), 8.03-8.01 (m, 2H), 7.53-7.48 (m, 4H), 6.45 (d, 1H, J=16.3 Hz), 3.93 (s, 3H);

¹³C NMR (125 MHz, CDCl3) δ 167.0, 142.4, 131.4, 129.5, 129.4, 129.0, 128.4, 126.9, 126.5, 125.5, 125.3, 52.1; IR (neat) 3051, 2949, 1719, 1635, 1435, 1265, 1170, 988, 886, 733; HRMS m/z calcd for C_{B}H₁₅O₂⁺ [M+H]⁺ 263.1067, observed 263.1074.

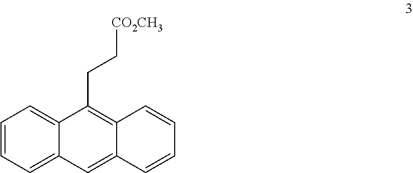 [SMILES: COC(=O)CCc1c2ccccc2cc2ccccc12]

### methyl 3-(anthracen-9-yl)propanoate (3)

1-3 To a solution of 2 (102 mg, 0.389 mmol) in DMF (5 mL) was added potassium formate (654 mg, 7.78 mmol) and Pd(OAc)₂ (4.4 mg, 0.02 mmol) and stirred at 60° C. for 4 h. After cooling, the mixture was filtered through Celite and washed with ethyl acetate. The filtrate was extracted with ethyl acetate and water. The combined organic layer was washed with water and brine, then dried over Na₂SO₄. The crude mixture was purified by column chromatography on silica gel (5% EtOAc/hexanes) to yield 3 (87.3 mg, 85%). ¹H NMR (500 MHz, CDCl3) δ 8.38 (s, 1H), 8.28 (dd, 2H, J=8.8, 0.6 Hz), 8.02 (dd, 2H, J=8.4, 0.5), 7.56-7.53 (m, 2H), 7.50-7.46 (m, 2H), 4.00-3.96 (m, 2H), 3.75 (s, 3H), 2.82-2.79 (m, 2H); ¹³C NMR (125 MHz, CDCl3) δ 173.6, 132.4, 131.7, 129.6, 129.6, 126.5, 126.1, 125.1, 124.0, 52.0, 35.2, 23.4; IR (neat) 3053, 2950, 1734, 1436, 1174, 885, 732; HRMS m/z calcd for C₁₈H₁₇O₂⁺ [M+H]⁺ 265.1223, observed 265.1226.

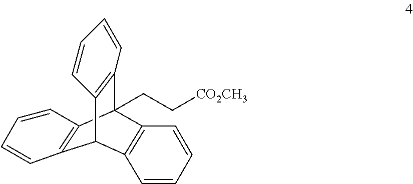 [SMILES: COC(=O)CCC12c3ccccc3C(c3ccccc31)c1ccccc12]

### methyl 3-(9,10-[1,2]benzenoanthracen-9(10H)-yl)propanoate (4)

4 To a solution of 3 (443 mg, 1.68 mmol) in acetonitrile (2.8 mL) was added CsF (764 mg, 5.03 mmol) and 2-(trimethylsilyl)phenyl trifluoromethanesulfonate (1.0 g, 3.35 mmol) and stirred at 80° C. for 4 h. Upon cooling, saturated NH₄Cl solution was added to the mixture and then extracted with dichloromethane. The combined organic layer was washed with brine and dried over Na₅SO₄. The crude mixture was purified by column chromatography on silica gel (5-10% EtOAc/hexanes) to yield 4 (542 mg, 95%). ¹H NMR (500 MHz, CDCl₃) δ 7.40-7.30 (m, 6H), 7.04-6.95 (m, 6H), 5.34 (s, 1H), 3.85 (s, 3H), 3.36-3.31 (m, 2H), 3.21-3.15 (m, 2H);

¹³C NMR (125 MHz, CDCl3) δ 174.7, 147.0, 125.2, 125.1, 123.8, 122.1, 54.6, 53.4, 52.2, 30.7, 22.7; IR (neat) 2952, 1733, 1450, 1176, 628; HRMS m/z calcd for C₂₄H₂₀NaO₂⁺ [M+Na]⁺ 363.1356, observed 363.1369.

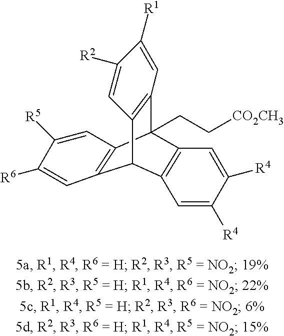

### methyl 3-(trinitro-9,10-[1,2]benzenoanthracen-9(10H)-yl)propanoate (5a-5d)

A solution of 4 (424.5 mg, 1.25 mmol) in concentrated HNO₃ (15 mL) was stirred at 75° C. overnight. The solution was cooled to room temperature, neutralized, and extracted with EtOAc. The organic layers were combined, washed with brine, and dried over Na₂SO₄. The crude mixture was then reesterified by stirring in methanol (50 mL) with catalytic H₂SO₄ under reflux overnight. The solution was concentrated under vacuum. Water was added and then basified by the addition of 1M NaOH. The water was extracted with EtOAc immediately. The organic layer was washed with brine and dried over Na₂SO₄. The crude mixture was purified by column chromatography on silica gel (30% EtOAc/hexanes) to give 5a (110 mg, 19%), 5b (130 mg, 22%), 5c (37.2 mg, 6.3%), and 5d (88.3 mg, 15%). 5a 1H NMR (500 MHz, CDCl₃) δ 8.32 (d, 2H, J=1.3 Hz), 8.28 (s, 1H), 8.01 (dd, 3H, J=8.3, 2.2 Hz), 7.69 (d, 1H, J=8.1 Hz), 7.62 (d, 2H, J=8.4 Hz), 5.87 (s, 1H), 3.91 (s, 3H), 3.52 (t, 2H, J=7.4 Hz), 3.17 (t, 2H, J=7.4 Hz); ¹³C NMR (125 MHz, CDCl₃) δ 173.4, 151.3, 150.2, 146.2, 146.1, 145.8, 145.0, 125.2, 123.6, 122.5, 122.1, 119.4, 118.3, 54.8, 53.3, 52.7, 30.2, 22.0; IR (neat) 2953, 1734, 1523, 1344, 1201, 738; HRMS m/z calcd for C₂₄H₁₈N₃NaO₈⁺ [M+Na]⁺ 498.0908, observed 498.0919; mp 143-146° C.; 5b ¹H NMR (500 MHz, CDCl₃) δ 8.32-8.27 (m, 3H), 8.04-7.97 (m, 3H), 7.69 (d, 2H, J=8.1 Hz), 7.64 (d, 1H, J=8.4 Hz), 5.88 (s, 1H), 3.92 (s, 3H), 3.56 (t, 2H, J=7.3 Hz), 3.20 (t, 2H, J=7.3 Hz); ¹³C NMR (125 MHz, CDCl3) δ 173.4, 150.9, 150.5, 146.2, 145.8, 145.7, 145.3 125.2, 123.6, 122.4, 122.1, 119.4, 118.1, 54.6, 53.4, 52.7, 30.2, 21.8; IR (neat) 3091, 2953, 2848, 1733, 1520, 1342, 1201, 736; HRMS m/z calcd for C₂₄H₁₈N₃NaO₈⁺ [M+Na]⁺ 498.0908, observed 498.0919; mp 139-142° C.; 5c ¹H NMR (500 MHz, CDCl3) δ 8.32 (d, 3H, J=2.2 Hz), 8.03 (dd, 3H, J=8.4, 2.2 Hz), 7.60 (d, 3H, J=8.4 Hz), 5.79 (s, 1H), 3.92 (s, 3H), 3.48 (t, 2H, J=7.6 Hz), 3.13 (t, 2H, J=7.6 Hz); ¹³C NMR (125 MHz, CDCl3) δ 173.4, 149.9, 146.7, 146.4, 123.7, 122.2, 119.5, 55.2, 53.6, 52.7, 30.5, 22.6; HRMS m/z calcd for C₂₄H₁₆N₃O₈ 474.0943, observed 474.0931; 5d ¹H NMR (500 MHz, CDCl3) δ 8.31 (d, 3H, J=1.7 Hz), 8.04 (dd, 3H, J=8.1, 1.8 Hz), 7.63 (d, 3H, J=8.1 Hz), 5.78 (s, 1H), 3.96 (s, 3H), 3.57 (t, 2H, J=7.3 Hz), 3.20 (t, 2H, J=7.3 Hz); ¹³C NMR (125 MHz, CDCl₃) δ 173.4, 150.5, 146.4, 125.2, 122.5, 118.3, 54.6, 53.8, 52.9, 30.3, 21.8; IR (neat) 3093, 2954, 2851, 1736, 1525, 1453, 1343, 1202, 1076, 903, 823; HRMS m/z calcd for C₂₄H₁₈N₃NaO₈⁺ [M+Na]⁺ 498.0908, observed [M+Na]⁺ 498.0910; mp 147-150° C.

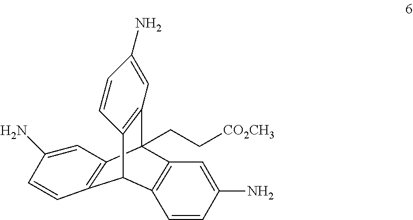 [SMILES: COC(=O)CCC12c3cc(N)ccc3C(c3ccc(N)cc31)c1ccc(N)cc12]

### methyl 3-(2,7,15-triamino-9,10-[1,2]benzenoanthracen-9(10H)-yl)propanoate (6)

To a solution of 5d (259 mg, 0.544 mmol) in methanol was added Pd/C (25 mg). The solution was purged with a H₂ gas balloon and kept under H₂ gas for 1 h. The mixture was filtered through Celite and washed with methanol. The filtrate was concentrated and purified by column chromatography on silica gel (5% MeOH/DCM) to give 6 (204 mg, 97%). ¹H NMR (500 MHz, MeOD) δ 7.01 (d, 3H, J=7.7 Hz), 6.79 (d, 3H, J=1.7 Hz), 6.33 (dd, 3H, J=7.7, 1.5 Hz), 4.98 (s, 1H), 3.85 (s, 3H), 3.18-3.13 (m, 4H); ¹³C NMR (125 MHz, MeOD) δ 176.5, 144.6, 140.7, 124.1, 112.6, 112.2, 54.2, 53.3, 52.5, 31.4, 23.8; IR (neat) 3354, 2951, 1724, 1605, 1473, 1326, 1181, 582 HRMS m/z calcd for C₂₄H₂₄N₃O₂⁺ [M+H]⁺ 386.1863, observed 386.1848.

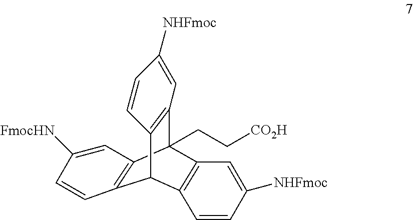

### 3-(2,7,15-tris((((9H-fluoren-9-yl)methoxy)carbonyl)amino)-9,10-[1,2]benzenoanthracen-9(10H)-yl)propanoic acid (7)

To a solution of 6 (116 mg, 0.301 mmol) in DCM (2.5 mL) was added excess pyridine. The solution was cooled to 0° C., then added Fmoc chloride in DCM (2.5 mL) slowly. The solution was allowed to warm to room temperature over time and stirred overnight. The mixture was extracted with DCM and acidic water. The organic layer was washed with brine and dried over MgSO₄. The crude mixture was purified by column chromatography on silica gel (30% EtOAc/hexanes). A solution of the ester (209 mg, 0.199 mmol) in dioxane (5 mL), H₂O (5 mL), and catalytic H₂SO₄ was stirred at 80° C. overnight. The reaction mixture was neutralized and concentrated under vacuum. Water was added to the mixture and was extracted with DCM. The organic layer was washed with brine and dried over Na₂SO₄. The crude mixture was purified by column chromatography on silica gel (50% EtOAc/hexanes) to yield 7 (165 mg, 80%). ¹H NMR (500 MHz, DMSO-d6) δ 12.44 (bs, 1H), 9.58 (s, 3H), 7.89 (d, 6H, J=7.6 Hz), 7.72 (d, 6H, J=7.0 Hz), 7.50 (s, 3H), 7.40 (t, 6H, J=7.4 Hz), 7.36-7.22 (m, 9H), 7.14 (bs, 3H), 5.37 (s, 1H), 4.44 (d, 6H, J=6.7 Hz), 4.28 (t, 3H, J=6.7 Hz), 3.13-2.92 (m, 4H); ¹³C NMR (125 MHz, DMSO) δ 174.2, 153.4, 143.7, 141.3, 140.7, 135.7, 127.6, 127.1, 125.1, 123.2, 120.1, 114.4, 113.5, 65.5, 52.2, 50.9, 46.6, 29.2, 22.2 IR (neat) 3375, 3325, 3075, 2950, 1709, 1605, 1528, 1450, 1212, 738; HRMS m/z calcd for C₆₉H₅₂N₃O₈⁺ [M−H]⁺ 1038.3749, observed 1038.3737; mp 169-171° C.

Solid Phase Synthesis:

All triptycenes were synthesized on 2-chlorotrityl chloride resin (100-200 mesh, 1.5 mmol substitution/g). The resin was added to a dry glass reaction vessel and swollen by stirring in dichloromethane (DCM) for 30 min. After swelling, the DCM was removed by vacuum and Fmoc-Trip-OH (8d) was coupled to the resin. Fmoc-Trip-OH (1.5 equiv) in 1:5 DMF:DCM and DIEA (5 equiv) were added and stirred for 5 min. DIEA (1.5 equiv) was added and the resin was stirred overnight. The solution was then drained by vacuum and the resin was washed thoroughly with DMF, then DCM, then DMF. The beads were deprotected by treatment with 20% piperidine in DMF for 1 h with stirring. The deprotection solution was removed by vacuum and the resin was washed thoroughly with DMF, DCM, then DMF. The first Fmoc-protected amino acid was then activated with HATU (9 equiv) in the presence of DIEA (18 equiv) prior to addition to the reaction vessel and allowed to couple overnight. Subsequent deprotections and amino acid couplings were run as described above. Before cleavage from the resin, the terminal Fmoc was removed. The beads were thoroughly washed with DMF then DCM. Peptides were cleaved by addition of trifluoroacetic acid (TFA), 2,2,2-trifluoroethanol (TFE), and DCM (9:1:1). The cleavage solution was collected by vacuum and concentrated using a rotary evaporator. The crude residue was diluted in 1:1 (0.1% TFA/H₂O:MeCN), purified by reverse-phase HPLC, and analyzed by MALDI-MS.

Fluorescence Quenching Assay: All experiments were conducted in 50 mM sodium phosphate buffer, pH 7.2. Fluorescence measurements were recorded with excitation at 495 nm and emission at 520 nm using 5 nm bandwidths on a Tecan M1000 plate reader. Inhibitor strand displacement by triptycene curves were obtained by incubating 120 nM TNR DNA with 10 μM inhibitor 10 for 2h, followed by addition of increasing concentrations of triptycenes. Samples were incubated for 2h and measured in triplicate in a 384-well plate.

Gel Shift Assay: Gel shift experiments were conducted in 50 mM sodium phosphate buffer, pH 7.2. Triptycene titration gels were prepared by incubating TNR 3WJ (0.5 μM) with inhibitor strand 10 (1.5 μM) for 2 h followed by titration of triptycenes and incubation at room temperature for 2h. Samples were loaded on a 20% non-denaturing polyacrylamide gel (19:1 monomer:bis) at 50V in 1×TBR buffer at 4° C. for 10 h. Gels were imaged by staining with SYBR Gold for 15 min then visualized using a BioRad GelDoc XR+ imager.

What is claimed is:

1. A method of screening for triptycene derivative (TCD) compounds that stabilize a target nucleic acid three way junction (TWJ) structures comprising:
a) providing an array comprising a solid support comprising a plurality of assay locations each comprising a covalently attached different TCD;
b) contacting said array with a target TWJ comprising:
i) a nucleic acid substrate with an attached fluorophore donor and an attached fluorophore acceptor; and
ii) a nucleic acid inhibitor that hybridizes to said substrate to form an inhibitor complex such that said donor and acceptor are separated and FRET does not occur, wherein said contacting is done under conditions wherein one of said TCDs binds to said TWJ such that said inhibitor is released and that FRET occurs; and
d) determining the binding of said TCD to said TWJ by detecting the presence or absence of FRET.

2. A method of screening for triptycene derivative (TCD) compounds that stabilize a target nucleic acid three way junction (TWJ) structures comprising:
a) providing a target nucleic acid substrate with an attached fluorophore donor and an attached fluorophore acceptor, said substrate forming a TWJ such that said donor and acceptor undergo fluorescence resonance energy transfer (FRET);
b) contacting said substrate with a nucleic acid inhibitor that hybridizes to said substrate to form an inhibitor complex such that said donor and acceptor are separated and FRET does not occur;
c) contacting said inhibited complex with a triptycene derivative (TCD) under conditions wherein said inhibitor is released and said TCD binds to said TWJ such that FRET reoccurs; and
d) determining the binding of said TCD to said TWJ by detecting the presence or absence of FRET.

3. A method of screening for triptycene derivative (TCD) compounds that stabilize nucleic acid three way junction (TWJ) structures comprising:
a) providing a nucleic acid substrate with an attached first fluorophore that forms a TWJ;
b) contacting said substrate with a nucleic acid inhibitor that hybridizes to said substrate to prevent the formation of the TWJ to form an inhibited complex, wherein said inhibitor comprises an attached second fluorophore, wherein said first and second fluorophore will undergo FRET when said inhibitor complex is formed;
c) contacting said inhibited complex with a TCD under conditions wherein said inhibitor is released, and said TCD binds to said TWJ such that FRET does not occur; and
d) determining the binding of said TCD to said TWJ by detecting the presence or absence of FRET.

4. A method according to claim 2 or 3 wherein said nucleic acid substrate is contacted with a plurality of TCDs.

5. A method according to any of claims 1 to 4 wherein said TCDs have the structure: 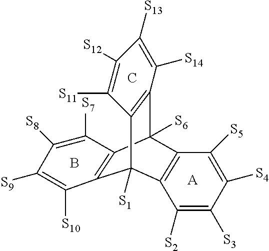
wherein each of S1 to S14 is independently and optionally selected from the group consisting of a hydrogen, substituted or unsubstituted alkyl, substituted or unsubstituted heteroalkyl, substituted or unsubstituted aryl, substituted or unsubstituted heteroaryl, halogen, CN, CF₃, acyl, an amino acid analog, a peptide (including peptide analogs), a nucleotide (including nucleotide analogs) and an oligonucleotide (including oligonucleotide analogs), and wherein at least one S group is a non-hydrogen group.

6. A method according to claim 5 wherein at least one of said S groups is an amino acid.

7. A method according to any of claims 5 to 6 wherein at least one of said S groups is an amino acid analog.

8. A method according to any of claims 5 to 7 wherein at least one of said S groups is a peptide.

9. A method according to any of claims 5 to 8 wherein at least one of said S groups is a peptide analog.

10. A method according to any of claims 5 to 9 wherein at least one of said S groups is a nucleotide.

11. A method according to any of claims 5 to 10 wherein at least one of said S groups is a nucleotide analog.

12. A method according to any of claims 5 to 11 wherein at least one of said S groups is an oligonucleotide.

13. A method according to any of claims 4 to 11 wherein at least one of said S groups is an oligonucleotide analog.

14. A method according to any of claims 2 to 12 wherein said TCD is covalently attached to a solid support.

15. A method according to claim 14 wherein a plurality of different TCDs are attached at different sites to said solid support in an array pattern.

16. A method of screening for a cytotoxic TCD comprising contacting said TCD with a cell and determining the viability of said cell.

17. A method according to claim 16 wherein said cell is a mammalian cell.

18. A method according to claim 16 wherein said TCD is contacted with a healthy cell or a cancerous cell.

19. A method according to claim 16 wherein said cell is a bacterial cell.

20. A method of screening for a TCD that inhibits viral replication comprising contacting a cell hosting a virus and determining the viability of said virus.

21. A composition comprising a solid support comprising an array of different TCDs.

22. A composition according to claim 21 wherein each TCD has the structure: 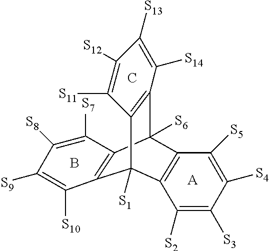
wherein each of S1 to S14 is independently and optionally selected from the group consisting of a hydrogen, substituted or unsubstituted alkyl, substituted or unsubstituted heteroalkyl, substituted or unsubstituted aryl, substituted or unsubstituted heteroaryl, halogen, CN, CF₃, acyl, an amino acid analog, a peptide (including peptide analogs), a nucleotide (including nucleotide analogs) and an oligonucleotide (including oligonucleotide analogs), and wherein at least one S group is used to covalently attached said TCD to said array.

23. A composition according to claim 21 or 22 wherein at least one of said S groups is attached via an amido group.

24. A composition according to any of claims 21 to 23 wherein at least one of said S groups is an amino acid.

25. A composition according to any of claims 21 to 24 wherein at least one of said S groups is an amino acid analog.

26. A composition according to any of claims 21 to 25 wherein at least one of said S groups is a peptide.

27. A composition according to any of claims 21 to 26 wherein at least one of said S groups is a peptide analog.

28. A composition according to any of claims 21 to 27 wherein at least one of said S groups is a nucleotide.

29. A composition according to any of claims 21 to 28 wherein at least one of said S groups is a nucleotide analog.

30. A composition according to any of claims 21 to 29 wherein at least one of said S groups is an oligonucleotide.

31. A composition according to any of claims 21 to 30 wherein at least one of said S groups is an oligonucleotide analog.
